SEQUENCE LISTING (155 sequences)

SEQ ID NO: 1 - DNA, 59 bp, synthetic construct
  modified_base 31: mod_base = i
  source 1..59: mol_type = other DNA
  misc_feature order(1..28,32..59): note = RNA
  misc_feature 29..31: note = DNA
  misc_feature 1..59: note = v117.26
catggccccagcagcttcagtccctttctcntcgatggtcagcacagccttatgcacgg

SEQ ID NO: 2 - DNA, 59 bp, synthetic construct; identical to SEQ ID NO: 1
  source 1..59: mol_type = other DNA
  misc_feature 1..59: note = v117.39
  modified_base 31: mod_base = i
  misc_feature order(1..28,32..59): note = RNA
  misc_feature 29..31: note = DNA

SEQ ID NO: 3 - DNA, 59 bp, synthetic construct; identical to SEQ ID NO: 1
  source 1..59: mol_type = other DNA
  misc_feature 1..59: note = v117.42
  modified_base 31: mod_base = i
  misc_feature order(1..3,6..9,12,15..18,21..28,32..33,36,39..40,43,45,48..51,53,55,57..59): note = RNA
  misc_feature order(4..5,10..11,13..14,19..20,29..31,34..35,37..38,41..42,44,46..47,52,54,56): note = DNA

SEQ ID NO: 4 - DNA, 59 bp, synthetic construct; identical to SEQ ID NO: 1
  source 1..59: mol_type = other DNA
  misc_feature 1..59: note = v117.43
  modified_base 31: mod_base = i
  misc_feature order(1..3,6..10,12..13,15..19,21..28,32..33,35..36,39..41,43..46,48..53,55..59): note = RNA
  misc_feature order(4..5,11,14,20,29..31,34,37..38,42,47,54): note = DNA

SEQ ID NO: 5 - DNA, 59 bp, synthetic construct; identical to SEQ ID NO: 1
  source 1..59: mol_type = other DNA
  misc_feature 1..59: note = v117.44
  modified_base 31: mod_base = i
  misc_feature order(1..9,11..12,14..18,20..28,32..34,36..40,42..43,45,47..51,53..55,57..59): note = RNA
  misc_feature order(10,13,19,29..31,35,41,44,46,52,56): note = DNA

SEQ ID NO: 6 - DNA, 59 bp, synthetic construct; identical to SEQ ID NO: 1
  source 1..59: mol_type = other DNA
  misc_feature 1..59: note = v117.45
  modified_base 31: mod_base = i
  misc_feature order(1..4,6..10,12..13,15..19,21..28,32..34,36..37,39..41,43..45,47..51,53..55,57..59): note = RNA
  misc_feature order(5,11,14,20,29..31,35,38,42,46,52,56): note = DNA

SEQ ID NO: 7 - DNA, 59 bp, synthetic construct; identical to SEQ ID NO: 1
  source 1..59: mol_type = other DNA
  misc_feature 1..59: note = v117.46
  modified_base 31: mod_base = i
  misc_feature order(1..3,5..9,11..12,14..18,20..28,32..33,35..36,38..40,42..43,45..46,48..53,55..59): note = RNA
  misc_feature order(4,10,13,19,29..31,34,37,41,44,47,54): note = DNA

SEQ ID NO: 8 - DNA, 59 bp, synthetic construct; identical to SEQ ID NO: 1
  source 1..59: mol_type = other DNA
  misc_feature 1..59: note = v117.47
  modified_base 31: mod_base = i
  misc_feature order(1..3,6..10,12..13,15..19,21..28,32..33,36,39..41,43..45,48..51,53,55,57..59): note = RNA
  misc_feature order(4..5,11,14,20,29..31,34..35,37..38,42,46..47,52,54,56): note = DNA

SEQ ID NO: 9 - DNA, 59 bp, synthetic construct; identical to SEQ ID NO: 1
  source 1..59: mol_type = other DNA
  misc_feature 1..59: note = v117.48
  modified_base 31: mod_base = i
  misc_feature order(1..3,6..10,12..13,15..19,21..28,32..33,36,39..40,43,45,48..51,53,55,57..59): note = RNA
  misc_feature order(4..5,11,14,20,29..31,34..35,37..38,41..42,44,46..47,52,54,56): note = DNA

SEQ ID NO: 10 - DNA, 59 bp, synthetic construct; identical to SEQ ID NO: 1
  source 1..59: mol_type = other DNA
  misc_feature 1..59: note = v117.49
  modified_base 31: mod_base = i
  misc_feature order(1..3,6..9,12,15..18,21..28,32..33,36,39..41,43,45..46,48..51,53,55,57..59): note = RNA
  misc_feature order(4..5,10..11,13..14,19..20,29..31,34..35,37..38,42,44,47,52,54,56): note = DNA

SEQ ID NO: 11 - DNA, 59 bp, synthetic construct; identical to SEQ ID NO: 1
  source 1..59: mol_type = other DNA
  misc_feature 1..59: note = v117.50
  modified_base 31: mod_base = i
  misc_feature order(1..3,6..10,11..12,14..19,21..28,32..33,36,39..41,43,45,48..51,53,55,57..59): note = RNA
  misc_feature order(4..5,11,14,20,29..31,34..35,37..38,42,44,46..47,52,54,56): note = DNA

SEQ ID NO: 12 - DNA, 59 bp, synthetic construct; identical to SEQ ID NO: 1
  source 1..59: mol_type = other DNA
  misc_feature 1..59: note = v117.51
  modified_base 31: mod_base = i
  misc_feature order(1..4,6..10,12..13,15..19,21..28,32..34,36..37,39..41,43,45..46,48..51,53,55,57..59): note = RNA
  misc_feature order(5,11,14,20,29..31,35,38,42,44,47,52,54,56): note = DNA

SEQ ID NO: 13 - DNA, 59 bp, synthetic construct; identical to SEQ ID NO: 1
  source 1..59: mol_type = other DNA
  misc_feature 1..59: note = v117.52
  modified_base 31: mod_base = i
  misc_feature order(1..3,5..9,11..12,14..18,20..28,32..33,35..36,38..40,42..43,45,47..51,53,55,57..59): note = RNA
  misc_feature order(4,10,13,19,29..31,34,37,41,44,46,52,54,56): note = DNA

SEQ ID NO: 14 - DNA, 59 bp, synthetic construct; identical to SEQ ID NO: 1
  source 1..59: mol_type = other DNA
  misc_feature 1..59: note = v117.53
  modified_base 31: mod_base = i
  misc_feature order(1..3,6..9,12,15..18,21..28,32..33,36,39..40,43..45,48..53,55..59): note = RNA
  misc_feature order(4..5,10..11,13..14,19..20,29..31,34..35,37..38,41..42,46..47,54): note = DNA

SEQ ID NO: 15 - DNA, 59 bp, synthetic construct; identical to SEQ ID NO: 1
  source 1..59: mol_type = other DNA
  misc_feature 1..59: note = v117.54
  modified_base 31: mod_base = i
  misc_feature order(1..3,6..9,12,15..18,21..28,32..33,36,39..40,43,45,48..51,53,55,57..59): note = RNA
  misc_feature order(4..5,10..11,13..14,19..20,29..31,34..35,37..38,41..42,44,46..47,52,54,56): note = DNA

SEQ ID NO: 16 - DNA, 59 bp, synthetic construct; identical to SEQ ID NO: 1
  source 1..59: mol_type = other DNA
  misc_feature 1..59: note = v117.55
  modified_base 31: mod_base = i
  misc_feature order(1..3,6..9,11..12,14..18,21..28,32..34,36..37,39..40,43,45,48..51,53,55,57..59): note = RNA
  misc_feature order(4..5,10,13,19..20,29..31,35,38,41..42,44,46..47,52,54,56): note = DNA

SEQ ID NO: 17 - DNA, 59 bp, synthetic construct; identical to SEQ ID NO: 1
  source 1..59: mol_type = other DNA
  misc_feature 1..59: note = v117.56
  modified_base 31: mod_base = i
  misc_feature order(1..3,6..9,12,14..19,21..28,32..34,36..37,39..40,43,45,48..51,53,55,57..59): note = RNA
  misc_feature order(4..5,10..11,13,20,29..31,35,38,41..42,44,46..47,52,54,56): note = DNA

SEQ ID NO: 18 - DNA, 59 bp, synthetic construct; identical to SEQ ID NO: 1
  source 1..59: mol_type = other DNA
  misc_feature 1..59: note = v117.57
  modified_base 31: mod_base = i
  misc_feature order(1..3,6..9,12,15..19,21..28,32..34,36..37,39..40,43,45,48..51,53,55,57..59): note = RNA
  misc_feature order(4..5,10..11,13..14,20,29..31,35,38,41..42,44,46..47,52,54,56): number = DNA

SEQ ID NO: 19 - DNA, 59 bp, synthetic construct; identical to SEQ ID NO: 1
  source 1..59: mol_type = other DNA
  misc_feature 1..59: note = v117.58
  modified_base 31: mod_base = i
  misc_feature order(1..3,6..10,12..13,15..19,21..28,32..34,36..37,39..40,43,45,48..51,53,55,57..59): note = RNA
  misc_feature order(4..5,11,14,20,29..31,35,38,41..42,44,46..47,52,54,56): note = DNA

SEQ ID NO: 20 - DNA, 59 bp, synthetic construct; identical to SEQ ID NO: 1
  source 1..59: mol_type = other DNA
  misc_feature 1..59: note = v117.59
  modified_base 31: mod_base = i
  misc_feature order(1..4,6..10,12..13,15..19,21..28,32..34,36..37,39..41,43..45,47..51,53..55,57..59): note = RNA
  misc_feature order(5,11,14,20,29..31,35,38,42,46,52,56): note = DNA

SEQ ID NO: 21 - DNA, 59 bp, synthetic construct; identical to SEQ ID NO: 1
  source 1..59: mol_type = other DNA
  misc_feature 1..59: note = v117.60
  modified_base 31: mod_base = i
  misc_feature order(1..3,6..10,12..13,15..19,21..28,32..34,36..37,39..40,43,45,48..51,53,55,57..59): note = RNA
  misc_feature order(4..5,11,14,20,29..31,35,38,41..42,44,46..47,52,54,56): note = DNA

SEQ ID NO: 22 - DNA, 59 bp, synthetic construct; identical to SEQ ID NO: 1
  source 1..59: mol_type = other DNA
  misc_feature 1..59: note = v117.62
  modified_base 31: mod_base = i
  misc_feature order(1..28,32..59): note = RNA
  misc_feature 29..31: note = DNA

SEQ ID NO: 23 - DNA, 59 bp, synthetic construct; identical to SEQ ID NO: 1
  source 1..59: mol_type = other DNA
  misc_feature 1..59: note = v117.63
  modified_base 31: mod_base = i
  misc_feature order(1..3,5..9,11..12,14..18,20..28,32..33,35..36,38..40,42..43,45..46,48..53,55..59): note = RNA
  misc_feature order(4,10,13,19,29..31,34,37,41,44,47,54): note = DNA

SEQ ID NO: 24 - DNA, 59 bp, synthetic construct; identical to SEQ ID NO: 1
  source 1..59: mol_type = other DNA
  misc_feature 1..59: note = v117.64
  modified_base 31: mod_base = i
  misc_feature order(1..3,6..10,12..13,15..19,21..28,32..33,35..36,39..41,43..46,48..53,55..59): note = RNA
  misc_feature order(4..5,11,14,20,29..31,34,37..38,42,47,54): note = DNA

SEQ ID NO: 25 - DNA, 59 bp, synthetic construct; identical to SEQ ID NO: 1
  source 1..59: mol_type = other DNA
  misc_feature 1..59: note = v117.65
  modified_base 31: mod_base = i
  misc_feature order(1..9,11..12,14..18,20..28,32..34,36..40,42..43,45,47..51,53..55,57..59): note = RNA
  misc_feature order(10,13,19,29..31,35,41,44,46,52,56): note = DNA

SEQ ID NO: 26 - DNA, 59 bp, synthetic construct; identical to SEQ ID NO: 1
  source 1..59: mol_type = other DNA
  misc_feature 1..59: note = v117.66
  modified_base 31: mod_base = i
  misc_feature order(1..4,6..10,12..13,15..19,21..28,32..34,36..37,39..41,43..45,47..51,53..55,57..59): note = RNA
  misc_feature order(5,11,14,20,29..31,35,38,42,46,52,56): note = DNA

SEQ ID NO: 27 - DNA, 59 bp, synthetic construct; identical to SEQ ID NO: 1
  source 1..59: mol_type = other DNA
  misc_feature 1..59: note = v117.75
  modified_base 31: mod_base = i
  misc_feature order(1..9,11..13,15..28,32..33,35..37,39..43,45..51,53..59): note = RNA
  misc_feature order(10,14,29..31,34,38,44,52): note = DNA

SEQ ID NO: 28 - DNA, 59 bp, synthetic construct; identical to SEQ ID NO: 1
  source 1..59: mol_type = other DNA
  misc_feature 1..59: note = v117.76
  modified_base 31: mod_base = i
  misc_feature order(1..4,6..9,11..12,15..19,21..28,32..33,35..36,39..41,43,45..46,48..51,53..55,57..59): note = RNA
  misc_feature order(5,10,13..14,20,29..31,34,37..38,42,44,47,52,56): note = DNA

SEQ ID NO: 29 - DNA, 59 bp, synthetic construct; identical to SEQ ID NO: 1
  source 1..59: mol_type = other DNA
  misc_feature 1..59: note = v117.77
  modified_base 31: mod_base = i
  misc_feature order(1..9,12..18,21..28,32..36,39..45,48..55,57..59): note = RNA
  misc_feature order(10..11,19..20,29..31,37..38,46..47,56): note = DNA

SEQ ID NO: 30 - DNA, 59 bp, synthetic construct; identical to SEQ ID NO: 1
  source 1..59: mol_type = other DNA
  misc_feature 1..59: note = V117.78
  modified_base 31: mod_base = i
  misc_feature order(1..28,32..33,36,39..40,43,45,48..51,53,55,57..59): note = RNA
  misc_feature order(29..31,34..35,37..38,41..42,44,46..47,52,54,56): note = DNA

SEQ ID NO: 31 - DNA, 59 bp, synthetic construct; identical to SEQ ID NO: 1
  source 1..59: mol_type = other DNA
  misc_feature 1..59: note = V117.79
  modified_base 31: mod_base = i
  misc_feature order(1..3,6..9,12,15..18,21..28,32..59): note = RNA
  misc_feature order(4..5,10..11,13..14,19..20,29..31): note = DNA

SEQ ID NO: 32 - DNA, 59 bp, synthetic construct; identical to SEQ ID NO: 1
  source 1..59: mol_type = other DNA
  misc_feature 1..59: note = v117.99
  modified_base 31: mod_base = i
  misc_feature order(1..5,7..9,11..13,15..17,19..21,23..25,27..28,32..34,36..38,40..42,44..46,48..50,52..54,56..59): note = RNA
  misc_feature order(6,10,14,18,22,26,29..31,35,39,43,47,51,55): note = DNA

SEQ ID NO: 33 - DNA, 59 bp, synthetic construct; identical to SEQ ID NO: 1
  source 1..59: mol_type = other DNA
  misc_feature 1..59: note = v117.100
  modified_base 31: mod_base = i
  misc_feature order(1..4,6,8,11..12,14..15,17..20,22..27,32..35,37..38,40..44,46..47,49..59): note = RNA
  misc_feature order(5,7,9..10,13,16,21,28..31,36,39,45,48): note = DNA

SEQ ID NO: 34 - DNA, 59 bp, synthetic construct
  source 1..59: mol_type = other DNA
  misc_feature 1..59: note = V117.28
  misc_feature order(1..28,32..59): note = RNA
  misc_feature 29..31: note = DNA
cagacacagaaatcaactcagtcttgatacatccagttcctttagggccatcaagttcc

SEQ ID NO: 35 - DNA, 59 bp, synthetic construct; identical to SEQ ID NO: 34
  source 1..59: mol_type = other DNA
  misc_feature 1..59: note = V117.29
  misc_feature order(1..28,32..59): note = RNA
  misc_feature 29..31: note = DNA

SEQ ID NO: 36 - DNA, 59 bp, synthetic construct; identical to SEQ ID NO: 34
  source 1..59: mol_type = other DNA
  misc_feature 1..59: note = V117.30
  misc_feature order(1..3,5,7,13..14,17..19,22..25,28,32..34,37..43,48..49,51..52,56..59): note = RNA
  misc_feature order(4,6,8..12,15..16,20..21,26..27,29..31,35..36,44..47,50,53..55): note = DNA

SEQ ID NO: 37 - DNA, 59 bp, synthetic construct; identical to SEQ ID NO: 34
  source 1..59: mol_type = other DNA
  misc_feature 1..59: note = V117.31
  misc_feature order(1..3,5,7..11,13..15,17..20,22..26,28,32..35,37..46,48..49,51..54,56..59): note = RNA
  misc_feature order(4,6,12,16,21,27,29..31,36,47,50,55): note = DNA

SEQ ID NO: 38 - DNA, 59 bp, synthetic construct; identical to SEQ ID NO: 34
  source 1..59: mol_type = other DNA
  misc_feature 1..59: note = V117.36
  misc_feature order(1..28,32..59): note = RNA
  misc_feature 29..31: note = DNA

SEQ ID NO: 39 - DNA, 59 bp, synthetic construct; identical to SEQ ID NO: 34
  source 1..59: mol_type = other DNA
  misc_feature 1..59: note = V117.37
  misc_feature order(1..4,6..10,12..13,15..19,21..28,32..34,36..37,39..41,43..45,47..51,53..55,57..59): note = RNA
  misc_feature order(5,11,14,20,29..31,35,38,42,46,52,56): note = DNA

SEQ ID NO: 40 - DNA, 59 bp, synthetic construct; identical to SEQ ID NO: 34
  source 1..59: mol_type = other DNA
  misc_feature 1..59: note = V117.38
  misc_feature order(1..5,7..8,10,12..14,16..19,21..25,27..28,32..34,36..43,45,47..49,51..53,55..59): note = RNA
  misc_feature order(6,9,11,15,20,26,29..31,35,44,46,50,54): note = DNA

SEQ ID NO: 41 - DNA, 59 bp, synthetic construct; identical to SEQ ID NO: 34
  misc_feature 1..59: note = V117.39
  source 1..59: mol_type = other DNA
  misc_feature order(1..3,5,7,9,13..14,17..19,21..26,28,32..34,36..43,45..49,51..52,55..59): note = RNA
  misc_feature order(4,6,8,10..12,15..16,20,27,29..31,35,44,50,53..54): note = DNA

SEQ ID NO: 42 - DNA, 59 bp, synthetic construct; identical to SEQ ID NO: 34
  misc_feature 1..59: note = V117.40
  source 1..59: mol_type = other DNA
  misc_feature order(1..8,10..20,22..25,27..28,32..35,37..44,48..54,56..59): note = RNA
  misc_feature order(9,21,26,29..31,36,45..47,55): note = DNA

SEQ ID NO: 43 - DNA, 59 bp, synthetic construct; identical to SEQ ID NO: 34
  source 1..59: mol_type = other DNA
  misc_feature 1..59: note = V117.41
  misc_feature order(1..28,32..59): note = RNA
  misc_feature 29..31: note = DNA

SEQ ID NO: 44 - DNA, 59 bp, synthetic construct
  source 1..59: mol_type = other DNA
  misc_feature 1..59: note = v117.20
  misc_feature order(1..28,32..59): note = RNA
  misc_feature 29..31: note = DNA
tttgtccattaaaaacagcatttgcaatacattcaaatccttgaagcaccggctggcac

SEQ ID NO: 45 - DNA, 59 bp, synthetic construct; identical to SEQ ID NO: 44
  source 1..59: mol_type = other DNA
  misc_feature 1..59: note = V117.22
  misc_feature order(1..7,9..14,16..17,19,21..23,25..26,28,32..36,38..45,47,49..51,53..55,57..59): note = RNA
  misc_feature order(8,15,18,20,24,27,29..31,37,46,48,52,56): note = DNA

SEQ ID NO: 46 - DNA, 59 bp, synthetic construct; identical to SEQ ID NO: 44
  source 1..59: mol_type = other DNA
  misc_feature 1..59: note = V117.23
  misc_feature order(1..28,32..59): note = RNA
  misc_feature 29..31: note = DNA

SEQ ID NO: 47 - DNA, 59 bp, synthetic construct; identical to SEQ ID NO: 44
  source 1..59: mol_type = other DNA
  misc_feature 1..59: note = V117.24
  misc_feature order(1..4,6..10,12..13,15..19,21..28,32..34,36..37,39..41,43..45,47..51,53..55,57..59): note = RNA
  misc_feature order(5,11,14,20,29..31,35,38,42,46,52,56): note = DNA

SEQ ID NO: 48 - DNA, 59 bp, synthetic construct; identical to SEQ ID NO: 44
  source 1..59: mol_type = other DNA
  misc_feature 1..59: note = V117.25
  misc_feature order(1..7,9..11,13,15..16,18..19,21..25,27..28,32..34,36,38..43,45,47..50,52..54,56..59): note = RNA
  misc_feature order(8,12,14,17,20,26,29..31,35,37,44,46,51,55): note = DNA

SEQ ID NO: 49 - DNA, 59 bp, synthetic construct
  source 1..59: mol_type = other DNA
  misc_feature 1..59: note = v117.20
  modified_base 31: mod_base = i
  misc_feature order(1..28,32..59): note = RNA
  misc_feature 29..31: note = DNA
ccccattctacagcagtactgagcaatgccntagtcagcaatctttgcaatgatggcag

SEQ ID NO: 50 - DNA, 59 bp, synthetic construct; identical to SEQ ID NO: 49
  source 1..59: mol_type = other DNA
  misc_feature 1..59: note = v117.42
  modified_base 31: mod_base = i
  misc_feature order(1..4,6..9,11,14,17,19..20,24,32,35..36,39,42..46,48,51,54,57..59): note = RNA
  misc_feature order(5,10,12..13,15..16,18,21..23,25..31,33..34,37..38,40..41,47,49..50,52..53,55..56): note = DNA

SEQ ID NO: 51 - DNA, 59 bp, synthetic construct; identical to SEQ ID NO: 49
  source 1..59: mol_type = other DNA
  misc_feature 1..59: note = v117.59
  modified_base 31: mod_base = i
  misc_feature order(1..9,11..12,14..15,17..20,22,24..25,27..28,32,34..36,38..39,41..46,48..49,51..52,54..55,57..59): note = RNA
  misc_feature order(10,13,16,21,23,26,29..31,33,37,40,47,50,53,56): note = DNA

SEQ ID NO: 52 - DNA, 59 bp, synthetic construct; identical to SEQ ID NO: 49
  misc_feature 1..59: note = v117.60
  source 1..59: mol_type = other DNA
  modified_base 31: mod_base = i
  misc_feature order(1..4,6..10,12..13,15..19,21..28,32..34,36..37,39..41,43..45,47..51,53..55,57..59): note = RNA
  misc_feature order(5,11,14,20,29..31,35,38,42,46,52,56): note = DNA

SEQ ID NO: 53 - DNA, 59 bp, synthetic construct
  source 1..59: mol_type = other DNA
  misc_feature 1..59: note = V117.21
  misc_feature order(1..28,32..59): note = RNA
  misc_feature 29..31: note = DNA
ggtactctcatatgtctttgactgatccacaatcttctggaacatggtccttttcttga

SEQ ID NO: 54 - DNA, 59 bp, synthetic construct; identical to SEQ ID NO: 53
  source 1..59: mol_type = other DNA
  misc_feature 1..59: note = V117.27
  misc_feature order(1..4,6..10,12..13,15..19,21..28,32..34,36..37,39..41,43..45,47..51,53..55,57..59): note = RNA
  misc_feature order(5,11,14,20,29..31,35,38,42,46,52,56): note = DNA

SEQ ID NO: 55 - RNA, 59 nt, synthetic construct
  source 1..59: mol_type = other RNA
  misc_feature 1..59: note = V117.19
ggtgctccttgatgagacgcagtcgtgtgcagcgtttgttgaggctcacactctcaccc

SEQ ID NO: 56 - DNA, 59 bp, synthetic construct
  source 1..59: mol_type = other DNA
  misc_feature 1..59: note = V117.20
  misc_feature order(1..4,6..10,12..13,15..19,21..28,32..34,36..37,39..41,43..45,47..51,53..55,57..59): note = RNA
  misc_feature order(5,11,14,20,29..31,35,38,42,46,52,56): note = DNA
ggtgctccttgatgagacgcagtcgtgtgcagcgtttgttgaggctcacactctcaccc

SEQ ID NO: 57 - DNA, 40 bp, synthetic construct
  source 1..40: mol_type = other DNA
  misc_feature 1..40: note = v121.10
  misc_feature order(1..33,37..40): note = RNA
  misc_feature 34..36: note = DNA
cggctccctaggcccctcccctcttcaaggggtccacatg

SEQ ID NO: 58 - DNA, 35 bp, synthetic construct
  source 1..35: mol_type = other DNA
  misc_feature 1..35: note = v121.11
  misc_feature order(1..28,32..35): note = RNA
  misc_feature 29..31: note = DNA
ccctaggcccctcccctcttcaaggggtccacatg

SEQ ID NO: 59 - DNA, 30 bp, synthetic construct
  source 1..30: mol_type = other DNA
  misc_feature 1..30: note = v121.12
  misc_feature order(1..21,25..30): note = RNA
  misc_feature 22..24: note = DNA
cccctcccctcttcaaggggtccacatggc

SEQ ID NO: 60 - DNA, 25 bp, synthetic construct
  source 1..25: mol_type = other DNA
  misc_feature 1..25: note = v121.13
  misc_feature order(1..16,20..25): note = RNA
  misc_feature 17..19: note = DNA
cccctcttcaaggggtccacatggc

SEQ ID NO: 61 - DNA, 50 bp, synthetic construct
  source 1..50: mol_type = other DNA
  misc_feature 1..50: note = V117.80
  modified_base 31: mod_base = i
  misc_feature order(1..4,6..10,12..13,15..19,21..28,32..34,36..37,39..41,43..45,47..50): note = RNA
  misc_feature order(5,11,14,20,29..31,35,38,41,46): note = DNA
catggccccagcagcttcagtccctttctcntcgatggtcagcacagcct

SEQ ID NO: 62 - DNA, 45 bp, synthetic construct
  source 1..45: mol_type = other DNA
  misc_feature 1..45: note = V117.81
  misc_feature order(1..4,6..10,12..13,15..19,21..28,32..34,36..37,39..41,43..45): note = RNA
  misc_feature order(5,11,14,20,29..31,35,38,42): note = DNA
  modified_base 31: mod_base = i
catggccccagcagcttcagtccctttctcntcgatggtcagcac

SEQ ID NO: 63 - DNA, 40 bp, synthetic construct
  source 1..40: mol_type = other DNA
  misc_feature 1..40: note = V117.82
  modified_base 31: mod_base = i
  misc_feature order(1..4,6..10,12..13,15..19,21..29,32..34,36..40): note = RNA
  misc_feature order(5,11,14,20,29..31,35): note = DNA
catggccccagcagcttcagtccctttctcntcgatggtc

SEQ ID NO: 64 - DNA, 31 bp, synthetic construct
  source 1..31: mol_type = other DNA
  misc_feature 1..31: note = V117.83
  modified_base 26: mod_base = i
  misc_feature order(1..5,7..8,10..14,16..23,27..29,31): note = RNA
  misc_feature order(6,9,15,24..26,30): note = DNA
ccccagcagcttcagtccctttctcntcgat

SEQ ID NO: 65 - DNA, 59 bp, synthetic construct; identical to SEQ ID NO: 1
  source 1..59: mol_type = other DNA
  misc_feature 1..59: note = V117.84
  modified_base 31: mod_base = i
  misc_feature order(1..28,32..59): note = RNA
  misc_feature 29..31: note = DNA

SEQ ID NO: 66 - DNA, 38 bp, synthetic construct
  source 1..38: mol_type = other DNA
  misc_feature 1..38: note = V117.85
  modified_base 31: mod_base = i
  misc_feature order(1..4,6..10,12..13,15..19,21..28,32..34,36..38): note = RNA
  misc_feature order(5,11,14,20,29..31,35): note = DNA
catggccccagcagcttcagtccctttctcntcgatgg

SEQ ID NO: 67 - DNA, 36 bp, synthetic construct
  source 1..36: mol_type = other DNA
  misc_feature 1..36: note = V117.86
  modified_base 31: mod_base = i
  misc_feature order(1..4,6..10,12..13,15..19,21..28,32..34,36): note = RNA
  misc_feature order(5,11,14,20,29..31,35): note = DNA
catggccccagcagcttcagtccctttctcntcgat

SEQ ID NO: 68 - DNA, 38 bp, synthetic construct
  source 1..38: mol_type = other DNA
  misc_feature 1..38: note = V117.87
  modified_base 29: mod_base = i
  misc_feature order(1..2,4..8,10..11,13..17,19..26,30..32,34..38): note = RNA
  misc_feature order(3,9,12,18,27..29,33): note = DNA
tggccccagcagcttcagtccctttctcntcgatggtc

SEQ ID NO: 69 - DNA, 35 bp, synthetic construct
  source 1..35: mol_type = other DNA
  misc_feature 1..35: note = V117.88
  modified_base 26: mod_base = i
  misc_feature order(1..5,7..8,10..14,16..23,27..29,31..35): note = RNA
  misc_feature order(6,9,15,24..26,30): note = DNA
ccccagcagcttcagtccctttctcntcgatggtc

SEQ ID NO: 70 - DNA, 38 bp, synthetic construct
  misc_feature 1..38: note = V117.89
  source 1..38: mol_type = other DNA
  modified_base 30: mod_base = i
  misc_feature order(1..3,5..9,11..12,14..18,20..27,31..33,35..38): note = RNA
  misc_feature order(4,10,13,19,28..30,34): note = DNA
atggccccagcagcttcagtccctttctcntcgatggt

SEQ ID NO: 71 - DNA, 36 bp, synthetic construct
  source 1..36: mol_type = other DNA
  misc_feature 1..36: note = V117.90
  modified_base 29: mod_base = i
  misc_feature order(1..2,4..8,10..11,13..17,19..26,30..32,34..36): note = RNA
  misc_feature order(3,9,12,18,27..29,33): note = DNA
tggccccagcagcttcagtccctttctcntcgatgg

SEQ ID NO: 72 - DNA, 34 bp, synthetic construct
  source 1..34: mol_type = other DNA
  misc_feature 1..34: note = V117.91
  modified_base 28: mod_base = i
  misc_feature order(1,3..7,9..10,12..16,18..25,29..31,33..34): note = RNA
  misc_feature order(2,8,11,17,26..28,32): note = DNA
ggccccagcagcttcagtccctttctcntcgatg

SEQ ID NO: 73 - DNA, 33 bp, synthetic construct
  source 1..33: mol_type = other DNA
  misc_feature 1..33: note = v117.141
  modified_base 28: mod_base = i
  misc_feature order(1,3..7,9..10,12..16,18..25,29..31,33): note = RNA
  misc_feature order(2,8,11,17,26..28,32): note = DNA
ggccccagcagcttcagtccctttctcntcgat

SEQ ID NO: 74 - DNA, 32 bp, synthetic construct
  source 1..32: mol_type = other DNA
  misc_feature 1..32: note = v117.142
  modified_base 27: mod_base = i
  misc_feature order(2..6,8..9,11..15,17..24,28..30,32): note = RNA
  misc_feature order(1,7,10,16,25..27,31): note = DNA
gccccagcagcttcagtccctttctcntcgat

SEQ ID NO: 75 - DNA, 31 bp, synthetic construct; identical to SEQ ID NO: 64
  source 1..31: mol_type = other DNA
  misc_feature 1..31: note = Block design_31nt
  modified_base 26: mod_base = i
  misc_feature order(1..23,27..31): note = RNA
  misc_feature 24..26: note = DNA

SEQ ID NO: 76 - DNA, 40 bp, synthetic construct; identical to SEQ ID NO: 63
  source 1..40: mol_type = other DNA
  misc_feature 1..40: note = Block design_40nt
  modified_base 31: mod_base = i
  misc_feature order(1..28,32..40): note = RNA
  misc_feature 29..31: note = DNA

SEQ ID NO: 77 - DNA, 33 bp, synthetic construct; identical to SEQ ID NO: 73
  source 1..33: mol_type = other DNA
  misc_feature 1..33: note = v117.167
  modified_base 28: mod_base = i
  misc_feature order(1..25,29..33): note = RNA
  misc_feature 26..28: note = DNA

SEQ ID NO: 78 - DNA, 33 bp, synthetic construct; identical to SEQ ID NO: 73
  source 1..33: mol_type = other DNA
  misc_feature 1..33: note = v117.168
  modified_base 28: mod_base = i
  misc_feature order(1..25,29..33): note = RNA
  misc_feature 26..28: note = DNA

SEQ ID NO: 79 - DNA, 33 bp, synthetic construct
  source 1..33: mol_type = other DNA
  misc_feature 1..33: note = v117.177
  modified_base 29: mod_base = i
  misc_feature order(1..2,4..8,10..11,13..17,19..26,30..32): note = RNA
  misc_feature order(3,9,12,18,27..29,33): note = DNA
tggccccagcagcttcagtccctttctcntcga

SEQ ID NO: 80 - DNA, 33 bp, synthetic construct
  source 1..33: mol_type = other DNA
  misc_feature 1..33: note = v117.178
  modified_base 27: mod_base = i
  misc_feature order(2..6,8..9,11..15,17..24,28..30,32..33): note = RNA
  misc_feature order(1,7,10,16,25..27,31): note = DNA
gccccagcagcttcagtccctttctcntcgatg

SEQ ID NO: 81 - DNA, 32 bp, synthetic construct; identical to SEQ ID NO: 74
  source 1..32: mol_type = other DNA
  misc_feature 1..32: note = v117.169
  modified_base 27: mod_base = i
  misc_feature order(1..24,28..32): note = RNA
  misc_feature 25..27: note = DNA

SEQ ID NO: 82 - DNA, 32 bp, synthetic construct; identical to SEQ ID NO: 74
  source 1..32: mol_type = other DNA
  misc_feature 1..32: note = v117.170
  modified_base 27: mod_base = i
  misc_feature order(1..24,28..32): note = RNA
  misc_feature 25..27: note = DNA

SEQ ID NO: 83 - DNA, 40 bp, synthetic construct
  source 1..40: mol_type = other DNA
  misc_feature 1..40: note = V117.42
  misc_feature order(1..8,10..20,22..25,27..28,32..35,37..40): note = RNA
  misc_feature order(9,21,26,29..31,36): note = DNA
cagacacagaaatcaactcagtcttgatacatccagttcc

SEQ ID NO: 84 - DNA, 40 bp, synthetic construct; identical to SEQ ID NO: 83
  source 1..40: mol_type = other DNA
  misc_feature 1..40: note = v117.44
  misc_feature order(1..28,32..40): note = RNA
  misc_feature 29..31: note = DNA

SEQ ID NO: 85 - DNA, 33 bp, synthetic construct
  source 1..33: mol_type = other DNA
  misc_feature order(1..5,7..17,19..22,24..25,29..33): note = RNA
  misc_feature order(6,18,23,26..28): note = DNA
  misc_feature 1..33: note = v117.56
acacagaaatcaactcagtcttgatacatccag

SEQ ID NO: 86 - DNA, 33 bp, synthetic construct; identical to SEQ ID NO: 85
  source 1..33: mol_type = other DNA
  misc_feature 1..33: note = v117.57
  misc_feature order(1..25,29..33): note = RNA
  misc_feature 26..28: note = DNA

SEQ ID NO: 87 - DNA, 59 bp, synthetic construct
  source 1..59: mol_type = other DNA
  misc_feature 1..59: note = V117.22
  modified_base 31: mod_base = i
  misc_feature order(1..28,32..59): note = RNA
  misc_feature 29..31: note = DNA
gcccttgccactcagggaaggagctgtggcngtggcaccagaatggattccagaatcca

SEQ ID NO: 88 - DNA, 40 bp, synthetic construct
  source 1..40: mol_type = other DNA
  misc_feature 1..40: note = V117.23
  modified_base 31: mod_base = i
  misc_feature order(1..28,32..40): note = RNA
  misc_feature 29..31: note = DNA
gcccttgccactcagggaaggagctgtggcngtggcacca

SEQ ID NO: 89 - DNA, 40 bp, synthetic construct; identical to SEQ ID NO: 88
  source 1..40: mol_type = other DNA
  misc_feature 1..40: note = v117.24
  modified_base 31: mod_base = i
  misc_feature order(1..9,11..13,15..17,20..21,23..28,32..36,38..40): note = RNA
  misc_feature order(10,14,18..19,22,29..31,37): note = DNA

SEQ ID NO: 90 - DNA, 40 bp, synthetic construct; identical to SEQ ID NO: 88
  source 1..40: mol_type = other DNA
  misc_feature 1..40: note = v117.25
  modified_base 31: mod_base = i
  misc_feature order(1..4,6..10,12..13,15..19,21..28,32..34,36..40): note = RNA
  misc_feature order(5,11,14,20,29..31,35): note = DNA

SEQ ID NO: 91 - DNA, 37 bp, synthetic construct
  source 1..37: mol_type = other DNA
  misc_feature 1..37: note = V117.29
  modified_base 31: mod_base = i
  misc_feature order(1..9,11..13,15..17,20..21,23..28,32..37): note = RNA
  misc_feature order(10,14,18..19,22,29..31): note = DNA
gcccttgccactcagggaaggagctgtggcngtggca

SEQ ID NO: 92 - DNA, 45 bp, synthetic construct
  source 1..45: mol_type = other DNA
  misc_feature 1..45: note = V120.2
  misc_feature order(1..33,37..45): note = RNA
  misc_feature 34..36: note = DNA
accgctttgtccattaaaaacagcatttgcaatacattcaaatcc

SEQ ID NO: 93 - DNA, 40 bp, synthetic construct
  source 1..40: mol_type = other DNA
  misc_feature 1..40: note = v117.28
  misc_feature order(1..7,9..11,13,15..16,18..19,21..25,27..28,32..34,36,38..40): note = RNA
  misc_feature order(8,12,14,17,20,26,29..31,35,37): note = DNA
tttgtccattaaaaacagcatttgcaatacattcaaatcc

SEQ ID NO: 94 - DNA, 40 bp, synthetic construct; identical to SEQ ID NO: 93
  source 1..40: mol_type = other DNA
  misc_feature 1..40: note = V117.26
  misc_feature order(1..4,6..10,12..13,15..19,21..28,32..34,36..40): note = RNA
  misc_feature order(5,11,14,20,29..31,35): note = DNA

SEQ ID NO: 95 - DNA, 40 bp, synthetic construct; identical to SEQ ID NO: 93
  source 1..40: mol_type = other DNA
  misc_feature 1..40: note = v117.27
  misc_feature order(1..3,5..17,19..23,25..28,32..40): note = RNA
  misc_feature order(4,18,24,29..31): note = DNA

SEQ ID NO: 96 - DNA, 45 bp, synthetic construct; identical to SEQ ID NO: 92
  source 1..45: mol_type = other DNA
  misc_feature 1..45: note = v117.29
  misc_feature order(1..3,5..8,10..22,24..28,30..33,37..45): note = RNA
  misc_feature order(4,9,23,29,34..36): note = DNA

SEQ ID NO: 97 - DNA, 40 bp, synthetic construct; identical to SEQ ID NO: 93
  source 1..40: mol_type = other DNA
  misc_feature 1..40: note = v117.39
  misc_feature order(1..28,32..40): note = RNA
  misc_feature 29..31: note = DNA

SEQ ID NO: 98 - DNA, 33 bp, synthetic construct
  misc_feature 1..33: note = v117.40
  source 1..33: mol_type = other DNA
  misc_feature order(1..25,29..33): note = RNA
  misc_feature 26..28: note = DNA
gtccattaaaaacagcatttgcaatacattcaa

SEQ ID NO: 99 - DNA, 33 bp, synthetic construct; identical to SEQ ID NO: 98
  source 1..33: mol_type = other DNA
  misc_feature 1..33: note = v117.41
  misc_feature order(2..14,16..20,22..25,29..33): note = RNA
  misc_feature order(1,15,21,26..28): note = DNA

SEQ ID NO: 100 - DNA, 32 bp, synthetic construct
  misc_feature 1..32: note = v117.42
  source 1..32: mol_type = other DNA
  misc_feature order(1..13,15..19,21..24,28..32): note = RNA
  misc_feature order(14,20,25..27): note = DNA
tccattaaaaacagcatttgcaatacattcaa

SEQ ID NO: 101 - DNA, 31 bp, synthetic construct
  source 1..31: mol_type = other DNA
  misc_feature 1..31: note = v117.43
  misc_feature order(1..12,14..18,20..23,27..31): note = RNA
  misc_feature order(13,19,24..26): note = DNA
ccattaaaaacagcatttgcaatacattcaa

SEQ ID NO: 102 - DNA, 38 bp, synthetic construct
  source 1..38: mol_type = other DNA
  misc_feature 1..38: note = v117.44
  misc_feature order(1..3,5..17,19..23,25..28,32..38): note = RNA
  misc_feature order(4,18,24,29..31): note = DNA
tttgtccattaaaaacagcatttgcaatacattcaaat

SEQ ID NO: 103 - DNA, 40 bp, synthetic construct; identical to SEQ ID NO: 83
  source 1..40: mol_type = other DNA
  misc_feature 1..40: note = v117.43
  misc_feature order(1..28,32..40): note = RNA
  misc_feature 29..31: note = DNA

SEQ ID NO: 104 - DNA, 40 bp, synthetic construct; identical to SEQ ID NO: 83
  source 1..40: mol_type = other DNA
  misc_feature 1..40: note = v117.45
  misc_feature order(1..8,10..28,32..35,37..40): note = RNA
  misc_feature order(9,29..31,36): note = DNA

SEQ ID NO: 105 - DNA, 40 bp, synthetic construct; identical to SEQ ID NO: 83
  source 1..40: mol_type = other DNA
  misc_feature 1..40: note = v117.46
  misc_feature order(1..20,22..25,27..28,32..40): note = RNA
  misc_feature order(21,26,29..31): note = DNA

SEQ ID NO: 106 - DNA, 40 bp, synthetic construct; identical to SEQ ID NO: 83
  source 1..40: mol_type = other DNA
  misc_feature 1..40: note = v117.47
  misc_feature order(1..25,27..28,32..35,37..40): note = RNA
  misc_feature order(26,29..31,36): note = DNA

SEQ ID NO: 107 - DNA, 40 bp, synthetic construct; identical to SEQ ID NO: 83
  source 1..40: mol_type = other DNA
  misc_feature 1..40: note = v117.48
  misc_feature order(1..8,10..20,22..28,32..40): note = RNA
  misc_feature order(9,21,29..31): note = DNA

SEQ ID NO: 108 - DNA, 40 bp, synthetic construct; identical to SEQ ID NO: 63
  source 1..40: mol_type = other DNA
  misc_feature 1..40: note = v117.101
  modified_base 31: mod_base = i
  misc_feature order(1..4,6..10,12..13,15..19,21..28,32..34,36..40): note = RNA
  misc_feature order(5,11,14,20,29..31,35): note = DNA

SEQ ID NO: 109 - DNA, 40 bp, synthetic construct; identical to SEQ ID NO: 63
  source 1..40: mol_type = other DNA
  misc_feature 1..40: note = v117.102
  modified_base 31: mod_base = i
  misc_feature order(1..4,6..10,12..13,15..19,21..28,32..34,36..40): note = RNA
  misc_feature order(5,11,14,20,29..31,35): note = DNA

SEQ ID NO: 110 - DNA, 40 bp, synthetic construct; identical to SEQ ID NO: 63
  source 1..40: mol_type = other DNA
  misc_feature 1..40: note = v117.103
  modified_base 31: mod_base = i
  misc_feature order(1..4,6..10,12..13,15..19,21..28,32..34,36..40): note = RNA
  misc_feature order(5,11,14,20,29..31,35): note = DNA

SEQ ID NO: 111 - DNA, 40 bp, synthetic construct; identical to SEQ ID NO: 63
  source 1..40: mol_type = other DNA
  misc_feature 1..40: note = v117.104
  modified_base 31: mod_base = i
  misc_feature order(1..4,6..10,12..13,15..19,21..28,34,36..40): note = RNA
  misc_feature order(5,11,14,20,29..33,35): note = DNA

SEQ ID NO: 112 - DNA, 40 bp, synthetic construct; identical to SEQ ID NO: 63
  source 1..40: mol_type = other DNA
  misc_feature 1..40: note = v117.105
  modified_base 31: mod_base = i
  misc_feature order(1..4,6..10,12..13,15..19,21..28,33..34,36..40): note = RNA
  misc_feature order(5,11,14,20,29..32,35): note = DNA

SEQ ID NO: 113 - DNA, 40 bp, synthetic construct; identical to SEQ ID NO: 63
  source 1..40: mol_type = other DNA
  misc_feature 1..40: note = v117.106
  modified_base 31: mod_base = i
  misc_feature order(1..4,6..10,12..13,15..19,21..28,32,34,36..40): note = RNA
  misc_feature order(5,11,14,20,29..31,33,35): note = DNA

SEQ ID NO: 114 - DNA, 40 bp, synthetic construct; identical to SEQ ID NO: 63
  source 1..40: mol_type = other DNA
  misc_feature 1..40: note = v117.118
  modified_base 31: mod_base = i
  misc_feature order(1..4,6..10,12..13,15..19,21..28,33..34,36..40): note = RNA
  misc_feature order(5,11,14,20,29..31,35): note = DNA
  misc_feature 32: note = oN = 2'-MOE

SEQ ID NO: 115 - DNA, 40 bp, synthetic construct; identical to SEQ ID NO: 63
  source 1..40: mol_type = other DNA
  misc_feature 1..40: note = v117.119
  modified_base 31: mod_base = i
  misc_feature order(1..4,6..10,12..13,15..19,21..28,33..34,36..40): note = RNA
  misc_feature order(5,11,14,20,29..31,35): note = DNA
  misc_feature 32: note = oN = 2'-MOE

SEQ ID NO: 116 - DNA, 40 bp, synthetic construct; identical to SEQ ID NO: 63
  source 1..40: mol_type = other DNA
  misc_feature 1..40: note = v117.120
  modified_base 31: mod_base = i
  misc_feature order(1..4,6..10,12..13,15..19,21..28,34,36..40): note = RNA
  misc_feature order(5,11,14,20,29..31,35): note = DNA
  misc_feature 32..33: note = oN = 2'-MOE

SEQ ID NO: 117 - DNA, 40 bp, synthetic construct; identical to SEQ ID NO: 63
  source 1..40: mol_type = other DNA
  misc_feature 1..40: note = v117.124
  modified_base 31: mod_base = i
  misc_feature order(1..4,6..10,12..13,15..19,21..28,32,34,36..40): note = RNA
  misc_feature order(5,11,14,20,29..31,35): note = DNA
  misc_feature 33: note = oN = 2'-MOE

SEQ ID NO: 118 - DNA, 59 bp, synthetic construct; identical to SEQ ID NO: 87
  source 1..59: mol_type = other DNA
  misc_feature 1..59: note = V117.19
  modified_base 31: mod_base = i
  misc_feature order(1..30,32..59): note = RNA
  misc_feature 31: note = DNA

SEQ ID NO: 119 - DNA, 59 bp, synthetic construct; identical to SEQ ID NO: 87
  source 1..59: mol_type = other DNA
  misc_feature 1..59: note = V117.20
  modified_base 31: mod_base = i
  misc_feature order(1..28,32..59): note = RNA
  misc_feature 29..31: note = DNA

SEQ ID NO: 120 - DNA, 59 bp, synthetic construct; identical to SEQ ID NO: 87
  source 1..59: mol_type = other DNA
  misc_feature 1..59: note = V117.21
  modified_base 31: mod_base = i
  misc_feature order(1..28,32..59): note = RNA
  misc_feature 29..31: note = DNA

SEQ ID NO: 121 - DNA, 59 bp, synthetic construct; identical to SEQ ID NO: 1
  source 1..59: mol_type = other DNA
  misc_feature 1..59: note = v117.68
  modified_base 31: mod_base = i
  misc_feature order(4,6..10,12..13,15..19,21..28,32..34,36..37,39..41,43..45,47..51,53..55): note = RNA
  misc_feature order(5,11,14,20,29..31,35,38,42,46,52,56): note = DNA
  misc_feature order(1..3,57..59): note = oN = 2'-MOE

SEQ ID NO: 122 - DNA, 40 bp, synthetic construct; identical to SEQ ID NO: 63
  source 1..40: mol_type = other DNA
  misc_feature 1..40: note = v117.137
  modified_base 31: mod_base = i
  misc_feature order(1..4,6..10,12..13,15..19,21..28,32..34,36..40): note = RNA
  misc_feature order(5,11,14,20,29..31,35): note = DNA

SEQ ID NO: 123 - DNA, 40 bp, synthetic construct; identical to SEQ ID NO: 63
  source 1..40: mol_type = other DNA
  misc_feature 1..40: note = v117.172
  modified_base 31: mod_base = i
  misc_feature order(4,6..10,12..13,15..19,21..28,32..34,36..37): note = RNA
  misc_feature order(5,11,14,20,29..31,35): note = DNA
  misc_feature order(1..3,38..40): note = oN = 2'-MOE

SEQ ID NO: 124 - DNA, 59 bp, synthetic construct; identical to SEQ ID NO: 1
  source 1..59: mol_type = other DNA
  misc_feature 1..59: note = v117.74
  modified_base 31: mod_base = i
  misc_feature order(1..4,6..10,12..13,15..19,21..28,32..34,36..37,39..41,43..45,47..51,53..55,57..59): note = RNA
  misc_feature order(5,11,14,20,29..31,35,38,42,46,52,56): note = DNA

SEQ ID NO: 125 - DNA, 59 bp, synthetic construct; identical to SEQ ID NO: 1
  source 1..59: mol_type = other DNA
  misc_feature 1..59: note = v117.71
  modified_base 31: mod_base = i
  misc_feature order(1..4,6..10,12..13,15..19,21..28,32..34,36..37,39..41,43..45,47..51,53..55,57..59): note = RNA
  misc_feature order(5,11,14,20,29..31,35,38,42,46,52,56): note = DNA

SEQ ID NO: 126 - DNA, 59 bp, synthetic construct; identical to SEQ ID NO: 1
  source 1..59: mol_type = other DNA
  misc_feature 1..59: note = v117.72
  modified_base 31: mod_base = i
  misc_feature order(1..4,6..10,12..13,15..19,21..28,32..34,36..37,39..41,43..45,47..51,53..55,57..59): note = RNA
  misc_feature order(5,11,14,20,29..31,35,38,42,46,52,56): note = DNA

SEQ ID NO: 127 - DNA, 59 bp, synthetic construct; identical to SEQ ID NO: 1
  source 1..59: mol_type = other DNA
  misc_feature 1..59: note = v117.73
  modified_base 31: mod_base = i
  misc_feature order(1..4,6..10,12..13,15..19,21..28,32..34,36..37,39..41,43..45,47..51,53..55,57..59): note = RNA
  misc_feature order(5,11,14,20,29..31,35,38,42,46,52,56): note = DNA

SEQ ID NO: 128 - DNA, 40 bp, synthetic construct; identical to SEQ ID NO: 63
  source 1..40: mol_type = other DNA
  misc_feature 1..40: note = v117.96
  modified_base 31: mod_base = i
  misc_feature order(1..4,6..10,12..13,15..19,21..28,32..34,36..40): note = RNA
  misc_feature order(5,11,14,20,29..31,35): note = DNA

SEQ ID NO: 129 - DNA, 40 bp, synthetic construct; identical to SEQ ID NO: 63
  source 1..40: mol_type = other DNA
  misc_feature 1..40: note = v117.107
  modified_base 31: mod_base = i
  misc_feature order(1..4,6..10,12..13,15..19,21..28,32..34,36..37,39..40): note = RNA
  misc_feature order(5,11,14,20,29..31,35,38): note = DNA

SEQ ID NO: 130 - DNA, 40 bp, synthetic construct; identical to SEQ ID NO: 63
  source 1..40: mol_type = other DNA
  misc_feature 1..40: note = v117.108
  modified_base 31: mod_base = i
  misc_feature order(1..4,6..10,12..13,15..19,21..28,32..34,36..37,39..40): note = RNA
  misc_feature order(5,11,14,20,29..31,35,38): note = DNA

SEQ ID NO: 131 - DNA, 40 bp, synthetic construct; identical to SEQ ID NO: 63
  source 1..40: mol_type = other DNA
  misc_feature 1..40: note = v117.109
  modified_base 31: mod_base = i
  misc_feature order(1..4,6..10,12..13,15..19,21..28,32..34,36..37,39..40): note = RNA
  misc_feature order(5,11,14,20,29..31,35,38): note = DNA

SEQ ID NO: 132 - DNA, 40 bp, synthetic construct; identical to SEQ ID NO: 63
  source 1..40: mol_type = other DNA
  misc_feature 1..40: note = v117.132
  modified_base 31: mod_base = i
  misc_feature order(1..4,6..10,12..13,15..19,21..28,32..34,36..37,39..40): note = RNA
  misc_feature order(5,11,14,20,29..31,35,38): note = DNA

SEQ ID NO: 133 - DNA, 40 bp, synthetic construct; identical to SEQ ID NO: 63
  source 1..40: mol_type = other DNA
  misc_feature 1..40: note = v117.133
  modified_base 31: mod_base = i
  misc_feature order(1..4,6..10,12..13,15..19,21..28,32..34,36..37,39..40): note = RNA
  misc_feature order(5,11,14,20,29..31,35,38): note = DNA

SEQ ID NO: 134 - DNA, 40 bp, synthetic construct; identical to SEQ ID NO: 63
  source 1..40: mol_type = other DNA
  misc_feature 1..40: note = v117.97
  modified_base 31: mod_base = i
  misc_feature order(2,4,6..10,12..13,15..19,21..28,32..34,36..37,39): note = RNA
  misc_feature order(5,11,14,20,29..31,35): note = DNA
  misc_feature order(1,3,38,40): note = lN = LNA

SEQ ID NO: 135 - DNA, 40 bp, synthetic construct; identical to SEQ ID NO: 63
  source 1..40: mol_type = other DNA
  misc_feature 1..40: note = v117.98
  modified_base 31: mod_base = i
  misc_feature order(1..4,6..10,12..13,15..19,21..23,25..28,32..34,37..38,40): note = RNA
  misc_feature order(5,11,14,20,29..31,35): note = DNA
  misc_feature order(24,36,39): note = lN = LNA

SEQ ID NO: 136 - DNA, 36 bp, synthetic construct; identical to SEQ ID NO: 67
  source 1..36: mol_type = other DNA
  misc_feature 1..36: note = v117.129
  modified_base 31: mod_base = i
  misc_feature order(2,4,6..10,12..13,15..19,21..28,32..34): note = RNA
  misc_feature order(5,11,14,20,29..31,35): note = DNA
  misc_feature order(1,3): note = lN = LNA

SEQ ID NO: 137 - DNA, 36 bp, synthetic construct; identical to SEQ ID NO: 67
  source 1..36: mol_type = other DNA
  misc_feature 1..36: note = v117.130
  modified_base 31: mod_base = i
  misc_feature order(2,4,6,8..10,12..13,15..19,21..28,32..34,36): note = RNA
  misc_feature order(11,14,20,29..31,35): note = DNA
  misc_feature order(1,3,5,7): note = lN = LNA

SEQ ID NO: 138 - DNA, 36 bp, synthetic construct; identical to SEQ ID NO: 67
  source 1..36: mol_type = other DNA
  misc_feature 1..36: note = v117.131
  modified_base 31: mod_base = i
  misc_feature order(2,4,6,8,10,12..13,15..19,21..28,32..34,36): note = RNA
  misc_feature order(14,20,29..31,35): note = DNA
  misc_feature order(1,3,5,7,9,11): note = lN = LNA

SEQ ID NO: 139 - DNA, 40 bp, synthetic construct; identical to SEQ ID NO: 63
  source 1..40: mol_type = other DNA
  misc_feature 1..40: note = v117.126
  modified_base 31: mod_base = i
  misc_feature order(2,4,6..10,12..13,15..19,21..28,32..34,36..37,39): note = RNA
  misc_feature order(5,11,14,20,29..31,35): note = DNA
  misc_feature order(1,3,38,40): note = lN = LNA

SEQ ID NO: 140 - DNA, 40 bp, synthetic construct; identical to SEQ ID NO: 63
  source 1..40: mol_type = other DNA
  misc_feature 1..40: note = v117.127
  modified_base 31: mod_base = i
  misc_feature order(2,4,6..10,12..13,15..19,21..28,32..34,36..37,39): note = RNA
  misc_feature order(5,11,14,20,29..31,35): note = DNA
  misc_feature order(1,3,38,40): note = lN = LNA

SEQ ID NO: 141 - DNA, 40 bp, synthetic construct; identical to SEQ ID NO: 63
  source 1..40: mol_type = other DNA
  misc_feature 1..40: note = v117.121
  modified_base 31: mod_base = i
  misc_feature order(1..28,32..40): note = RNA
  misc_feature 29..31: note = DNA

SEQ ID NO: 142 - DNA, 40 bp, synthetic construct; identical to SEQ ID NO: 63
  source 1..40: mol_type = other DNA
  misc_feature 1..40: note = v117.123
  modified_base 31: mod_base = i
  misc_feature order(1..28,32..40): note = RNA
  misc_feature 29..31: note = DNA

SEQ ID NO: 143 - DNA, 40 bp, synthetic construct; identical to SEQ ID NO: 63
  source 1..40: mol_type = other DNA
  misc_feature 1..40: note = v117.152
  modified_base 31: mod_base = i
  misc_feature order(1..28,32..40): note = RNA
  misc_feature 29..31: note = DNA

SEQ ID NO: 144 - DNA, 40 bp, synthetic construct; identical to SEQ ID NO: 63
  source 1..40: mol_type = other DNA
  misc_feature 1..40: note = v117.153
  modified_base 31: mod_base = i
  misc_feature order(1..28,32..40): note = RNA
  misc_feature 29..31: note = DNA

SEQ ID NO: 145 - DNA, 40 bp, synthetic construct; identical to SEQ ID NO: 63
  source 1..40: mol_type = other DNA
  misc_feature 1..40: note = v117.154
  modified_base 31: mod_base = i
  misc_feature order(1..28,32..40): note = RNA
  misc_feature 29..31: note = DNA

SEQ ID NO: 146 - DNA, 40 bp, synthetic construct; identical to SEQ ID NO: 63
  source 1..40: mol_type = other DNA
  misc_feature 1..40: note = v117.155
  modified_base 31: mod_base = i
  misc_feature order(1..28,32..40): note = RNA
  misc_feature 29..31: note = DNA

SEQ ID NO: 147 - DNA, 40 bp, synthetic construct; identical to SEQ ID NO: 63
  source 1..40: mol_type = other DNA
  misc_feature 1..40: note = v117.158
  modified_base 31: mod_base = i
  misc_feature order(1..28,32..40): note = RNA
  misc_feature 29..31: note = DNA

SEQ ID NO: 148 - DNA, 40 bp, synthetic construct; identical to SEQ ID NO: 63
  source 1..40: mol_type = other DNA
  misc_feature 1..40: note = v117.160
  modified_base 31: mod_base = i
  misc_feature order(1..28,32..40): note = RNA
  misc_feature 29..31: note = DNA

SEQ ID NO: 149 - DNA, 40 bp, synthetic construct; identical to SEQ ID NO: 83
  source 1..40: mol_type = other DNA
  misc_feature 1..40: note = v117.53
  misc_feature order(1..28,32..40): note = RNA
  misc_feature 29..31: note = DNA

SEQ ID NO: 150 - DNA, 40 bp, synthetic construct; identical to SEQ ID NO: 83
  source 1..40: mol_type = other DNA
  misc_feature 1..40: note = v117.54
  misc_feature order(1..28,32..40): note = RNA
  misc_feature 29..31: note = DNA

SEQ ID NO: 151 - DNA, 40 bp, synthetic construct; identical to SEQ ID NO: 83
  source 1..40: mol_type = other DNA
  misc_feature 1..40: note = v117.55
  misc_feature order(1..28,32..40): note = RNA
  misc_feature 29..31: note = DNA

SEQ ID NO: 152 - DNA, 45 bp, synthetic construct; identical to SEQ ID NO: 92
  source 1..45: mol_type = other DNA
  misc_feature 1..45: note = v117.30
  misc_feature order(1..3,5..8,10..22,24..28,30..33,37..45): note = RNA
  misc_feature order(4,9,23,29,34): note = DNA
  misc_feature 35..36: note = aN = 2' FANA

SEQ ID NO: 153 - DNA, 45 bp, synthetic construct; identical to SEQ ID NO: 92
  source 1..45: mol_type = other DNA
  misc_feature 1..45: note = v117.31
  misc_feature order(1..3,5..8,10..22,24..28,30..32,37..45): note = RNA
  misc_feature order(4,9,23,29): note = DNA
  misc_feature 33..36: note = aN = 2' FANA

SEQ ID NO: 154 - DNA, 50 bp, synthetic construct
  source 1..50: mol_type = other DNA
  misc_feature 1..50: note = v117.33
  misc_feature order(1..34,39..50): note = RNA
  misc_feature 35..38: note = aN = 2' FANA
tgaccgctttgtccattaaaaacagcatttgcaatacattcaaatccttg

SEQ ID NO: 155 - DNA, 59 bp, synthetic construct; identical to SEQ ID NO: 1
  source 1..59: mol_type = other DNA
  misc_feature 1..59: note = v117.59 + Amino
  modified_base 31: mod_base = i
  misc_feature order(1..4,6..10,12..13,15..19,21..28,32..34,36..37,39..41,43..45,47..51,53..55,57..59): note = RNA
  misc_feature order(5,11,14,20,29..31,35,38,42,46,52,56): note = DNA

(19) United States
(12) Patent Application Publication
(10) Pub. No.: US 2025/0101418 A1
(43) Pub. Date: Mar. 27, 2025

(54) CHEMICALLY MODIFIED ANTISENSE OLIGONUCLEOTIDES (ASOS) AND COMPOSITIONS COMPRISING THE SAME FOR RNA EDITING

(71) Applicant: Eberhard-Karls-Universität-Tübingen, Tübingen (DE)

(72) Inventors: Thorsten Stafforst, Tübingen (DE); Laura Sophia Pfeiffer, Ostfildern (DE); Ngadhnjim Latifi, Tübingen (DE)

(21) Appl. No.: 18/565,415

(22) PCT Filed: Nov. 30, 2022
(86) PCT No.: PCT/EP2022/083943

§ 371 Date: Nov. 29, 2023

(51) Int. Cl. C12N 15/113

(52) U.S. Cl. CPC C12N 15/113; C12N 2310/11; C12N 2310/315; C12N 2310/321; C12N 2310/322; C12N 2310/3231

(57) ABSTRACT

The invention relates to chemically modified oligonucleotides comprising a sequence with a length of 23 to 80 nucleotides, capable of binding to a target sequence in a target RNA, comprising a central base triplet (CBT) of 3 nucleotides (5′-N₋₁^{e}N₀^{f}N₋₁^{g}-3′) with the central nucleotide (N₀) directly opposite to the target adenosine in the target RNA, wherein the core oligonucleotide comprises the following sequence: 5′-N₋₅^{a}N₋₄^{b}N₋₃^{c}N₋₂^{d}N₋₁^{e}N₀^{f} N₊₁^{g}N₊₂^{h}N₊₃ⁱN₊₄^{j}-3′ comprising different 2′ sugar and linkage modifications. The present disclosure also provides oligonucleotides and compositions thereof for use in use in the treatment or prevention of a genetic disorder, condition, or disease. Also provided are methods for editing a target adenosine or deaminating at least one specific adenosine in a target nucleic acid.

### FIELD OF THE INVENTION

The present invention relates to the field of medicine, in particular to the field of site-directed RNA editing, whereby an RNA sequence is targeted by a single-stranded antisense oligonucleotide (ASO) for RNA editing of a particular genetic mutation (“compensatory editing”) or for editing of an RNA derived from a wildtype allele (“beneficial editing”).

### BACKGROUND OF THE INVENTION

RNA editing is a natural process through which some cells can make discrete changes to specific nucleotide sequences within an RNA molecule in a site-specific way. Unlike DNA editing, the advantage of RNA editing is that it allows modification of the genetic information in a more efficient manner. This is because RNAs are generally quickly degraded, any errors introduced by off-target modifications would be washed out, rather than permanently staying with the modified DNA of the subject. RNA editing may also be less likely to cause an immune reaction because it is an editing mechanism that is naturally found in humans. Moreover, RNA editing might provide a more natural response than introducing an external, engineered gene.

Over the years, oligonucleotide therapeutics have been developed to specifically silence, restore, or modify the expression of disease-causing or disease-associated genes in, e.g., cancer and (other) genetic disorders. Such therapeutics include, for example, antisense oligonucleotides (ASOs), small interfering RNA (siRNA) and microRNA that interfere with coding and noncoding RNA. The relative ease and accuracy with which ASO sequences can be customized allows virtually any mutated gene to be targeted. As a result, ASOs are the most clinically developed, with several drugs already approved by the U.S. Food and Drug Administration (FDA) and in clinical trials (Cideciyan et al., 2019; Gagliardi and Ashizawa, 2021).

Generally, a vast group of proteins are involved in mediating the intracellular RNA editing process (Quinones-Valdez et al., 2019). Specifically, Site-Directed RNA Editing (SDRE) describes the alteration of an RNA sequence by introducing or removing nucleotides from an RNA or by changing the character of a nucleobase by deamination. RNA editing enzymes are known in the art. The first RNA editing process discovered in mammals was the deamination of cytidine (C) by APOBEC proteins to form uridine (U) (Zinshteyn and Nishikura, 2009). To date, the two most useful and most studied types of RNA editing are cytidine (C) to uridine (U) (“C-to-U”) and adenosine (A) to inosine (1) (“A-to-I”) conversions.

Notably, for therapeutic purposes and the most prevalent type of RNA editing in higher eukaryotes is the “A-to-I” conversion. This conversion is catalysed by the adenosine deaminases acting on RNA (ADARs) family. Over the years, three vertebrate ADAR genes have been identified, which give rise to several ADAR proteins that result from alternative promoters or represent splice variants (Wulff and Nishikura, 2010). These proteins are expressed across various types of human tissues, and which can alter splicing and translation machineries, double-stranded RNA (dsRNA) structures and the binding affinity between RNA and RNA-binding proteins (Tomaselli et al., 2014; Zinshteyn and Nishikura, 2009). Of the three known ADAR genes, hADAR1 and hADAR2 are expressed in most tissues and encode active deaminases. Human ADAR3 (hADAR3) has been described to only be expressed in the central nervous system and reportedly has no deaminase activity in vitro. While all ADARs are multidomain proteins, comprising a targeting or dsRNA-binding domain (dsRBD) and a catalytic domain, ADAR1 proteins additionally comprise one or more Z binding domains, while splice variant ADAR2R and ADAR3 comprises an R domain (Zinshteyn and Nishikura, 2009; Wulff and Nishikura, 2010). Accordingly, in some embodiments, the ADAR is hADAR1, hADAR2 or hADAR3.

“A-to-I” editing was initially identified in Xenopus eggs (Bass and Weintraub, 1987; Rebagliati and Melton, 1987). Human cDNA encoding “double stranded RNA adenosine deaminase” was first cloned by Kim et al. (1994) and the adenosine to inosine (“A-to-I”) conversion activity of the protein confirmed by recombinant expression in insect cells. “A-to-I” editing changes the informational content of the RNA molecule, as inosine preferentially base pairs with cytidine and is therefore interpreted as guanosine (G) by the translational and splicing machinery. During this enzymatically catalyzed reaction adenosine is changed via a hydrated intermediate to inosine. While guanosine can form three hydrogen bonds to the complementary base cytidine, inosine can form only two hydrogen bonds to cytidine. The translational machinery reads inosine as a guanosine. Therefore, ADARs have the effect of introducing a functional adenosine to guanosine mutation on the RNA level. The ability of ADARs to alter the sequence of RNAs has also been used to artificially target RNAs in vitro in cells for RNA editing. Potentially this approach may be used to repair genetic defects and alter genetic information at the RNA level.

ASOs are generally short, in the range of 18 to 25 nucleobases in length, single-stranded synthetic RNA or DNA molecules, which use Watson-Crick base pairing to bind sequence specifically to the target RNA. They can be broadly classified into 1^{st}, 2^{nd} and 3^{rd} generation ASOs. The first ASOs were employed to inhibit translation of Rous sarcoma virus ribosomal RNA (Stephenson and Zamecnik, 1978). While 1^{st} generation ASOs are characterised in having a modified backbone, wherein the nucleotide linkages are modified by sulphur, methyl or amine groups to generate phosphorothioates (PS), methyl-phosphonates, and phosphoramidates, respectively, 2^{nd} generation ASOs additionally carry alkyl modifications at the 2′ position of the ribose. These 2^{nd} generation ASOs tend to be less toxic than PS-modified ASOs and have a slightly higher affinity for their target. In comparison, 3^{rd} generation ASOs tend to be even more heterogenous as they include a large number of chemical modifications that aim to improve binding-affinity, stability, and pharmacokinetics (Quemener et al., 2019). The diversity of chemical modifications, together with the sequence of the ASO, offers considerable flexibility as relates to the therapeutic approach. That is, depending on their mechanism of action, ASOs can be used to degrade target mRNA to decrease protein levels, to modify or correct splicing events, to modulate RNA translation or to target pathological coding or non-coding RNAs (Quemener et al., 2019).

ASOs can work through many mechanisms depending, in part, on the region in the RNA sequence that is targeted and ASO design/chemical properties. To ensure ASO specificity, their sequences are ideally complementary or at least partially complementary to the target RNA. However, in the case of site-directed mutagenesis, i.e., “A-to-I” RNA editing, the ASO targeting domain contains a mismatch opposite the targeted adenosine. It is to be noted that several endogenous substrates of ADAR contain mismatches and/or bulges (Thomas and Beal, 2017) and therefore could alter or even improve substrate recognition, if these features are mimicked in the ASO/resulting dsRNA.

Furthermore, ASOs can be chemically modified to improve their properties. For instance, ASOs can be modified to protect them against nucleases and to increase their effectiveness. While phosphorothioate (PS) modifications seem to have a positive effect on ASOs stability and pharmacokinetics, the difference in chirality of PS linkages may have a substantial influence on the ASO's overall property. PS linkages can be found in two stereoisomers, Rp and Sp, and it is known from the art, that Rp and Sp linkages can influence properties such as, e.g., thermal stability, binding affinity, pharmacologic properties, etc., of the ASO. However, the benefit of Rp and Sp stereoisomers has been controversial (Iwamoto et al., 2017; Crooke et al., 2020).

While the use of antisense oligonucleotides in RNA editing is generally known in the art (Vogel et al., 2014; Merkle et al., 2019), ASO-based therapies have been gaining more traction over the past years for the treatment of different medical conditions and diseases, in particular genetic disorders. RNA editing systems employing endogenous adenosine deaminase enzymes have been extensively studied, i.e., the use of exogenous oligonucleotides to specifically recruit endogenous adenosine deaminases to a specific target site of a target RNA thereby providing an improved system for targeted RNA editing. Oligonucleotide constructs for site-directed RNA editing are described in patent applications WO 2016/097212 and WO 2017/010556, which utilise endogenous cellular pathways, i.e., endogenous ADAR, to edit endogenous RNA. Using structural information available for ADAR-RNA complexes new designs for nucleoside analogues are constantly being investigated (Doherty et al., 2021). The use of endogenous ADAR-mediated RNA editing in non-human primates using stereopure chemically modified oligonucleotides has previously been reported (Monian et al., 2022). These oligonucleotides typically are very rich in 2′-F-modifications within the 5′ half, which are generally present as blocks of 2′-F-modifications and uniform block of 2′-O-Methyl-modifications within the 3′ terminus on either side of the CBT. Furthermore, these oligonucleotides contain massively stereopure PS-modified backbone and massively stereopure PS linkages and additional charge-neutral PN linkage (also stereopure), the latter of which is not yet applied in the clinics. That precise, site-specific RNA editing can be achieved by recruiting endogenous ADARs with antisense oligonucleotides has previously been shown by Merkle et al. (2019). Merkle et al. (2019) were able to demonstrate that chemically optimized ASOs can be used to recruit endogenous human ADARs to edit endogenous transcripts in a simple and programmable way with almost no off-target editing.

In WO 2020/001793, the inventors of the instant application provided for an artificial nucleic acid for site-directed editing of RNA (“A-to-I” editing), wherein the nucleic acid comprises a targeting sequence and recruiting moiety. Similarly, WO 2018/041973 relates to ASOs that can bring about specific editing of adenosines in a target RNA sequence, wherein said ASO does, however, not form an intramolecular hairpin or stem-loop structure. Specifically, WO2018/041973 relates to chemically modified single-stranded RNA-editing oligonucleotides for the deamination of a target adenosine by an ADAR enzyme whereby the central base triplet (CBT) of three sequential nucleotides comprises a sugar modification and/or a base modification. It was found that deoxyribose at all three positions of the CBT is well tolerated and provides substantial stabilization against nuclease digestion.

Other prior art, including WO 2021/071858 relates to oligonucleotides comprising a first and second domain, wherein the first domain comprises one or more 2′-F modifications, and wherein the second domain comprises one or more sugars that do not have a 2′-F modification. WO 2022/099159 relates to oligonucleotides with a first and second domain, wherein the domains comprise specific percentages of 2′-F modifications and aliphatic substitutions.

Research in the field of ASO optimisation has led to the identification and more thorough investigation of the CBT and the immediate 5′ and 3′ region around it. In addition to specifically looking at CBT modifications (e.g., 2′-F and 2′-FANA), WO 2021/243023 also mentions guide or targeting domain modifications 3′ to the nucleobase just outside the CBT (at position +2 of an oligonucleotide comprising the structure [Am]-X¹-X²-X³-X⁴-[Bn], wherein X⁴ corresponds to the +2 position). It was found that the selection of nucleotide at the +2 position of the triplet of the guide oligonucleotide can affect the editing rate of the target. Improved editing was observed with a 2′-F modification at the +2 position.

However, despite being a promising technology, few ASOs have been marketed. This is due to difficulties pertaining to stability, cellular delivery and uptake, and clinical efficacy, as well as off-target effects and/or preclinical toxicologic challenges. Hence, to ultimately translate ASO-based therapies into a widespread clinical success, it is crucial to overcome these different challenges. Accordingly, there is currently an unmet need for improved ASOs and effective therapies for the treatment of genetic disorders involving these improved ASOs.

The inventors have discovered that the artificial and chemically modified oligonucleotides of the invention are suitable for editing a wide variety of endogenous RNA transcripts, e.g., endogenous mRNAs of housekeeping genes as well as endogenous transcripts of disease-related genes such as, e.g., STAT1, SERPINA 1, LRRK2, CRB1, NLRP3, CTNNB1, PEX1, and PDE6A. Surprisingly, the inventors have discovered that the instant invention provides improved ASOs. For example, the ASOs of the instant application have improved editing efficacies and potencies. Furthermore, the ASOs of the instant application provide the advantage of having an increased lysosomal half-life, i.e., improved lysosomal stability, and facilitating ease of production (e.g., cost, purity, quality control). The ASOs of the invention also have the advantage of decreasing off-target edits of RNA.

### SUMMARY OF THE INVENTION

Therefore, the problem solved by the instant application lies in the provision of improved synthetic and chemically modified ASOs capable of mediating a functional change from an adenosine (A) to a guanosine (G) to correct point mutations, which otherwise have a deleterious effect. The present invention solves this problem by providing synthetic ASOs that comprise specific nucleoside modifications, specifically at the 2′ position of the sugar, backbone linkage modifications and combinations thereof. Overall, the ASOs of the instant application show several differences and associated advantages over those disclosed in the prior art. Above all, the inventive character of the inventions lies in the specific combination of nucleoside and linkage modifications provided herein. To date, no prior art has been identified that teaches or suggests the oligonucleotides, compositions, and methods as disclosed in the present application, which are all based on the inventors' discovery that certain combinations of nucleobase, nucleoside and linkage modifications are particularly effective in providing stable and effective ASOs and compositions comprising the same.

The solution to the problem addressed by the instant application is achieved by the embodiments described herein and defined by the appended claims.

The present invention generally provides oligonucleotides and compositions comprising said oligonucleotides and their use in the treatment or prevention of a genetic disorder, condition, or disease. Also provided herein is an in vitro method for editing a target adenosine in a target nucleic acid and an in vitro method for deaminating at least one specific adenosine present in a target RNA sequence in a cell. Also provided here are methods of treating or preventing genetic disorder, condition, or disease, wherein the method comprises administering an effective amount of the oligonucleotides of the invention.

In a first aspect, the present invention provides a chemically modified oligonucleotide comprising a sequence with a length of 23 to 80 nucleotides, capable of binding to a target sequence in a target RNA, comprising a central base triplet (CBT) of 3 nucleotides (5′-N₋₁^{e}N₀^{f}N₊₁^{g}-3′) with the central nucleotide (N₀) directly opposite to the target adenosine in the target RNA, wherein the core oligonucleotide comprises the following sequence:
5′-N₋₅^{a}N₋₄^{b} N₋₃^{c}N₋₂^{d}N₋₁^{e}N₀^{f}N₊₁^{g}N₊₂^{h}N₊₃ⁱN₊₄^{j}-3′; and wherein:
(a) at least two of the three nucleotides of the CBT are chemically modified at the 2′ position of the sugar moiety, are deoxyribonucleosides, or a combination thereof and wherein d and e are internucleoside linkage modifications;
(b) the N₊₂ nucleotide carries a 2′-O-alkyl-modification; and wherein the N₊₃ nucleotide carries a 2′-fluoro (2′-F)-modification;
(c) at least 10% of nucleotides are 2′-F-modified and at least 10% of nucleotides are 2′-O-alkyl-modified, wherein no more than 6 consecutive nucleotides have the same 2′-modification;
(d) the internucleoside linkage modification content is at least 15%; and
(e) linkages h and i are not phosphorothioate (PS) linkages.

In a second aspect provided herein is a chemically modified oligonucleotide comprising a sequence with a length of 23 to 50 nucleotides, capable of binding to a target sequence in a target RNA, comprising a central base triplet (CBT) of 3 nucleotides (5′-N₋₁^{e}N₀^{f}N₊₁^{g}-3′) with the central nucleotide (N₀) directly opposite to the target adenosine in the target RNA, wherein the core oligonucleotide comprises the following sequence:
5′-N₋₅^{a}N₋₄^{b} N₋₃^{c}N₋₂^{d}N₋₁^{e}N₀^{f}N₊₁^{g}N₊₂^{h}N₊₃ⁱN₊₄^{j}-3′; and wherein:
(a) at least two of the three nucleotides of the CBT are chemically modified at the 2′ position of the sugar moiety, are deoxyribonucleosides, or a combination thereof and wherein d and e are internucleoside linkage modifications;
(b) the N₊₂ nucleotide carries a 2′-O-alkyl-modification; and wherein the N₊₃ nucleotide carries a 2′-fluoro (2′-F)-modification;
(c) at least 10% of nucleotides are 2′-F-modified and at least 10% of nucleotides are 2′-O-alkyl-modified, wherein no more than 6 consecutive nucleotides have the same 2′-modification;
(d) the regions 3′ and 5′ to the CBT do not contain more than a total of 6 deoxyribonucleosides;
(e) the internucleoside linkage modification content is at least 30%.

In a third aspect provided herein is a chemically modified oligonucleotide comprising a sequence with a length of 40 to 80 nucleotides, capable of binding to a target sequence in a target RNA, comprising a central base triplet (CBT) of 3 nucleotides (5′-N₋₁^{e}N₀^{f}N₊₁^{g}-3′) with the central nucleotide (N₀) directly opposite to the target adenosine in the target RNA, wherein the core oligonucleotide comprises the following sequence: 5′-N₋₅^{a}N₋₄^{b} N₋₃^{c}N₋₂^{d}N₋₁^{e}N₀^{f}N₊₁^{g}N₊₂^{h}N₊₃ⁱN₊₄^{j}-3′; and wherein:
(a) at least two of the three nucleotides of the CBT are chemically modified at the 2′ position of the sugar moiety, are deoxyribonucleosides, or a combination thereof and wherein d and e are internucleoside linkage modifications;
(b) the N₊₂ nucleotide carries a 2′-O-alkyl-modification; and wherein the N₊₃ nucleotide carries a 2′-fluoro (2′-F)-modification;
(c) at least 10% of nucleotides are 2′-F-modified and at least 10% of nucleotides are 2′-O-alkyl-modified, wherein no more than 6 consecutive nucleotides have the same 2′-modification;
(d) the regions 3′ and 5′ to the CBT have a total deoxyribonucleoside content of 5-50%.

In a fourth aspect provided herein is a pharmaceutical composition comprising the oligonucleotide of the invention or a pharmaceutically acceptable salt thereof.

In a fifth aspect provided herein is a chemically modified oligonucleotide of the invention or a pharmaceutical composition of the invention for use in the treatment or prevention of a genetic disorder, condition, or disease.

In a sixth aspect provided herein is an in vitro method for editing a target adenosine in a target nucleic acid, wherein the method comprises contacting the target nucleic acid with the oligonucleotide of the invention.

In a seventh aspect provided herein is an in vitro method for deaminating at least one specific adenosine present in a target RNA sequence in a cell, wherein the method comprises the steps of:
(a) contacting the target nucleic acid with a chemically modified oligonucleotide of the invention;
(b) allowing uptake by the cell of the chemically modified oligonucleotide;
(c) allowing annealing of the chemically modified oligonucleotide to the target RNA sequence; and
(d) allowing a mammalian ADAR enzyme comprising a natural dsRNA binding domain as found in the wild type enzyme to deaminate the target adenosine in the target RNA sequence to an inosine.

### BRIEF DESCRIPTION OF DRAWINGS

The figures shown in the following are merely illustrative and shall describe the present invention in a further way. The figures shall not be construed to limit the present invention thereto.

FIG. 1 presents graphs showing the editing efficacy and lysosomal stability of SERPINA targeting oligonucleotides.

FIG. 2 presents a graph showing the SERPINA editing efficacy of 2′-F-modified oligonucleotides.

FIG. 3 presents graphs showing the editing efficacy and lysosomal stability of STAT1 Y701 targeting oligonucleotides.

FIG. 4 presents graphs showing editing efficacy and lysosomal stability of CRB1 C948Y targeting oligonucleotides.

FIG. 5 presents graphs showing the editing efficacy and lysosomal stability of LRRK2 G2019S targeting oligonucleotides.

FIG. 6 presents a graph showing the editing efficacy of murine PDE6A (mPDE6A) V685M targeting oligonucleotides.

FIG. 7 presents graphs showing the editing efficacy of NLRP3 Y166 targeting oligonucleotides expressed in plasmid (A) and genomically integrated (B).

FIG. 8 presents a graph showing data on the editing efficacy of GAPDH 3′UTR targeting oligonucleotides.

FIG. 9 presents a graph showing data on the editing efficacy of truncated variants (31 nt, 40 nt, 45 nt, 50 nt, 59 nt) of SERPINA targeting oligonucleotides.

FIG. 10 presents data on the editing efficacy of 5′ and/or 3′ truncated variants of SERPINA targeting oligonucleotides.

FIG. 11 represents a graph showing the editing efficacy of 32 nt and 33 nt long SERPINA targeting oligonucleotides.

FIG. 12 presents a graph on the editing efficacy of 3′ terminus truncated STAT1 Y701 targeting oligonucleotides.

FIG. 13 represents a graph showing the editing efficacy of 3′ terminus truncated CTNNB1 T41 targeting oligonucleotides.

FIG. 14 presents graphs showing the editing efficacy of 5′ and/or 3′ terminus truncated CRB1 C948Y targeting oligonucleotides.

FIG. 15 presents a graph showing the editing efficacy of STAT1 Y701 targeting oligonucleotides.

FIG. 16 presents a graph showing the editing efficacy of SERPINA targeting oligonucleotide with modifications in the extended hotspot region 3′ to the CBT (optimal version: +2 (2′-OMe) and +3 (2′-F)).

FIG. 17 presents graphs showing the editing efficacy of CTNNB1 T41 targeting oligonucleotides with modifications in the extended hotspot region 3′ to the CBT (optimal version: +2 (2′-OMe) and +3 (2′-F)).

FIG. 18 presents graphs showing the editing efficacy, lysosomal stability, and relative toxicity of SERPINA targeting oligonucleotides containing 2′-MOE endblocks.

FIG. 19 presents graphs showing the editing efficacy and lysosomal stability of long SERPINA targeting oligonucleotides (59 nt) with decreased PS linkages using the GENOMIC (A) and PLASMID (B) systems.

FIG. 20 presents a graph showing data on the editing efficacy of short SERPINA targeting oligonucleotides (40 nt) with decreased PS linkage modifications.

FIG. 21 presents a graph showing the editing efficacy of SERPINA targeting oligonucleotides that contain continuous stretches of PS linkages.

FIG. 22 presents graphs showing the impact of LNA modifications on the editing efficacy and potency of SERPINA targeting oligonucleotides.

FIG. 23 presents a graph showing the impact of LNA modifications at the 5′ terminus of SERPINA targeting oligonucleotides.

FIG. 24 presents graphs showing 5′ terminus LNA modifications of short SERPINA targeting oligonucleotides.

FIG. 25 presents graphs showing 5′ and 3′ termini block disruption of SERPINA targeting oligonucleotides.

FIG. 26 presents graphs showing block disruption of short, STAT1 targeting oligonucleotides.

FIG. 27 represents graphs showing 2′-FANA modification of the CBT of CRB1 C948Y targeting oligonucleotides.

### DETAILED DESCRIPTION

### Terminology

In order that the present invention may be more readily understood, certain terms are first defined.

The articles “a” and “an” are used herein to refer to one or to more than one (i.e., to at least one) of the grammatical object of the article. By way of example, “an element” means one element or more than one element, e.g., a plurality of elements.

The terms “about” and “approximately” may be understood to permit standard variation as would be understood by those of ordinary skill in the art.

The term “including” is used herein to mean, and is used interchangeably with, the phrase “including, but not limited to”. Likewise, the term “comprising” is used herein to mean, and is used interchangeably with, the phrase “comprising, but not limited to”.

As used herein, the term “nucleic acid” is intended to include any DNA molecules (e.g., cDNA or genomic DNA) and any RNA molecules (e.g., mRNA) and analogues of the DNA or RNA generated using nucleotide analogues. For example, in one embodiment, the oligonucleotide comprises, e.g., an UNA (unlocked nucleic acid), a PMO (phosphorodiamidate linked morpholino) or a PNA (peptide nucleic acid). The nucleic acid can be single-stranded or double-stranded. Oligonucleotides can be single-stranded (ss) or double-stranded (ds). A single-stranded oligonucleotide can have double-stranded regions (formed by two portions of the single-stranded oligonucleotide) and a double-stranded oligonucleotide, which comprises two oligonucleotide chains, can have single-stranded regions for example, at regions where the two oligonucleotide chains are not complementary to each other. Each component of the DNA or RNA structure can be modified and be categorized by modification of (1) the internucleoside linkage, (2) the deoxyribose/ribose, and/or (3) the nucleobase.

The term “oligonucleotide” or “oligonucleotides” as used herein are defined as it is generally understood by the skilled person as a molecule including two or more covalently linked nucleosides (e.g., short nucleic acid polymer(s)). They can comprise DNA and/or RNA.

The oligonucleotides provided herein have a backbone comprising deoxyribonucleotides and/or ribonucleotides.

The term “nucleobase” refers to nitrogen-containing biological building blocks that form nucleosides, which, in turn, are components of nucleotides. The naturally occurring bases [guanine, (G), adenine, (A), cytosine, (C), thymine, (T), and uracil (U)] are derivatives of purine or pyrimidine, though it should be understood that naturally and non-naturally occurring base analogues are also included and that the term “nucleobase” also includes “modified nucleobases”.

Within the context of this invention, the term “modified nucleobase” and “modified base” may be used interchangeably with the term “nucleobase”. Nucleobases may be modified or unmodified. Hence, in some embodiments, a modified nucleobase is a nucleobase which comprises a modification. In some embodiments, a modified nucleobase is capable of at least one function of a nucleobase, e.g., forming a moiety in a polymer capable of base-pairing to a nucleic acid comprising an at least complementary sequence of bases. In one embodiment, the modified nucleobase is capable of increasing hydrogen bonding, base pair stacking interactions and/or stabilizing a nucleic acid complex. In another embodiment, the modified nucleobase (e.g., Benner's base) is capable of mimicking the N3 protonated cytosine base. In some embodiments, a modified nucleobase is substituted A, T, C, G, or U, or a substituted tautomer of A, T, C, G, or U. In some embodiments, a modified nucleobase in the context of oligonucleotides refer to a nucleobase that is not A, T, C, G or U. Modifications include but are not limited to nonstandard nucleobases 5-methyl-2′-deoxycytidine (m⁵C), pseudouridine (pU), dihydrouridine, inosine (I), and 7-methylguanosine. Other modifications may include nucleobase replacement by (N) heterocycles (e.g., nebularine) or aromatic rings that stack well in the RNA duplex, such as, e.g., a Benner's base Z (and/or analogues) or 8-oxo-adenosine (8-oxo-A). As used herein, the term “Benner's base Z” refers to the pyrimidine analogue 6-amino-5-nitro-3-(1′-β-D-2′-deoxyribofuranosyl)-2(1H)-pyridone (dZ). In one embodiment, a modification includes the introduction of nucleobase analogues or simple heterocycles that boost editing. As used herein, and as commonly understood by the skilled person in the art, the expression “derivative thereof” refers to a derivative of a (modified) nucleobase, nucleoside or nucleotide. For example, a derivative may be a corresponding nucleobase, nucleoside or nucleotide that has been chemically derived from said nucleobase, nucleoside or nucleotide. For instance, a derivative of deoxycytidine may include fluoro-modified deoxycytidine, 5-methyl-2′-deoxycytidine (m⁵C), or ribocytidine.

The term “nucleoside(s)” refers to a moiety wherein a nucleobase or a modified nucleobase is covalently bound to a sugar or a modified sugar. In some embodiments, a “nucleoside” refers to a nucleoside unit in an oligonucleotide or a nucleic acid. The term “nucleoside(s)” encompasses all modified versions and derivatives “modified nucleobases”.

The term “nucleotide(s)” as used herein refers to a monomeric unit of a polynucleotide that consists of a nucleobase, a sugar, and one or more linkages (e.g., phosphate linkages in natural DNA and RNA). In some cases, the linkage may be a non-naturally occurring and/or modified linkage. In some embodiments, the linkage may be an internucleoside linkage as described herein. In one specific embodiment, the modified linkage is a PS linkage. In some embodiments, a “nucleotide” refers to a nucleotide unit in an oligonucleotide or a nucleic acid. The term “nucleotide(s)” encompasses all modified versions and derivatives of “nucleosides” and “modified nucleobases”.

As used herein, the term “internucleoside linkage” refers to a linkage between adjacent nucleosides. “Internucleoside linkage” and “linkage” may be used interchangeably. Linkages may be continuous or consecutive. Linkages may be discontinuous or interrupted. As used herein, the term “discontinuous” or “interrupted” means that there are not more than, e.g., 4, 5, 6, 7 or more consecutive internucleoside linkage modifications of the same modification. In some embodiments, the naturally occurring PO linkages are replaced by modified internucleoside linkages. Hence, in some embodiments, the linkage is a non-natural internucleoside linkage. In some embodiments, internucleoside linkage(s) include, but are not limited to phosphorothioate (PS), 3′-methylenephosphonate, 5′-methylenephosphonate, 3′-phosphoroamidate, 2′-5′-phosphodiester, and phosphoryl guanidine (PN) linkages. In another embodiment, the internucleoside linkage modification is a 3′-3′ or 5′-5′ phosphate ester bonds (3′-P-3′ and 5′-P-5′). The internucleoside linkage may be stereopure or stereorandom. Thus, within a particular oligonucleotide, internucleoside linkages may comprise stereopure and stereorandom linkages. In one embodiment, the natural 3′-5′ phosphodiester linkage is replaced by modified internucleoside linkages. In some embodiments, the naturally occurring one or more PO linkages are replaced by modified internucleoside linkages in order to introduce one or more PS linkages or non-phosphorus derived internucleoside linkages.

As used herein the term “stereopure” or “stereorandom” refers to chemically modified oligonucleotides. Specifically, the term “stereopure” refers to oligonucleotides that are chirally pure (or “stereochemically pure”). The term “stereorandom” refers to racemic (or “stereorandom”, “non-chirally controlled”) oligonucleotides. Hence, the oligonucleotides of the invention comprise 1, 2, 3, 4, 5, 6, 7, 8, 9, 10 or more stereorandom internucleoside linkages (mixture of Rp and Sp linkage phosphorus at the internucleoside linkage, e.g., from traditional non-chirally controlled oligonucleotide synthesis). In one embodiment, an internucleoside linkage is a phosphorothioate (PS) linkage. In one embodiment, an internucleoside linkage is a stereorandom PS linkage. In one embodiment, an internucleoside linkage is a chirally controlled PS linkage. In one embodiment, an internucleoside linkage is a phosphoryl guanidine (PN) linkage.

The term “hydroxy”, as used herein, represents an —OH group.

As used herein the term “antisense oligonucleotide” or “ASO” refers to a short strand of nucleotide analogue that hybridizes with the complementary mRNA in a sequence-specific manner via Watson-Crick base pairing. The ASO can comprise DNA and RNA. The ASO may be chemically modified. As used herein the terms “antisense oligonucleotide” (ASO) and “oligonucleotide” may be used interchangeably.

The term “modified sugar” refers to a moiety that can replace a naturally occurring sugar. A modified sugar mimics the spatial arrangement, electronic properties, or some other physicochemical property of a sugar. The naturally occurring sugar is the pentose (five carbon sugar) deoxyribose (to form DNA) or ribose (to form RNA), though it should be understood that naturally and non-naturally occurring sugar analogues are also included. For example, other sugars may comprise, e.g., C4 sugars, C5 sugars and/or C6 sugars. In some embodiments, a modified sugar is substituted ribose or deoxyribose. In some embodiments, a modified sugar comprises a 2′-modification. Examples of useful 2′-sugar modifications, e.g., 2′-ribose, 2′-deoxyribose, 2′-arabinose etc., are widely used in the art and described herein. Those skilled in the art, after reading the present disclosure, will appreciate that various types of 2′-sugar modifications are known and can be utilized in accordance with the present disclosure. In some embodiments, a modified sugar is a bicyclic sugar (e.g., a sugar used in LNA, BNA, etc.). In some embodiments, a modified sugar is an LNA sugar. The term “locked nucleic acid” (LNA) or term “locked nucleic acids” (LNAs) are also known as bridged nucleic acid (BNA) and refers to modified RNA nucleotide in which the ribose moiety is modified with an extra bridge connecting the 2′ oxygen and 4′ carbon. In some embodiments, a sugar modification is 2′-OMe, 2′-O-methoxy-ethyl (2′-MOE), 2′-F, 5′-vinyl, or S-constrained ethyl (S-cEt). In one embodiment, a 2′-modification is a C2-stereoisomer of 2′-F-ribose. In one embodiment, a 2′-modification is 2′-F. In one embodiment, a 2′-modification is 2′-FANA. In one embodiment, a modified sugar is a sugar of morpholino. In one embodiment, the oligonucleotide comprises, e.g., an UNA (unlocked nucleic acid), a PMO (phosphorodiamidate linked morpholino) or a PNA (peptide nucleic acid). Hence, in one embodiment, the nucleic acid analogue is a PNA (peptide nucleic acid). In one embodiment, the nucleic acid analogue is PMO (phosphorodiamidate linked morpholino). In one embodiment, a 2′-modification is a 2′-O-alkyl modification. In one embodiment, the 2′-O-alkyl modification is a 2′-O-methyl-, 2′-O-ethyl-, 2′-O-propyl-, or 2′-MOE modification. In a preferred embodiment, a 2′-modification is 2′-OMe. In some embodiments, a 2′-modification is 2′-MOE. In some embodiments, a 2′-modification is 2′-OR, wherein R is substituted C1-10 aliphatic. In some embodiments, in the context of oligonucleotides, a modified sugar is a sugar that is not ribose or deoxyribose as typically found in natural RNA or DNA (e.g., arabinose). In some instances, the 2′-O-alkyl modification is not a 2′-MOE.

The term “FANA” or “FANA-modified” refers to 2′-fluoroarabinoside modified nucleobases and/or oligonucleotides comprising such nucleobases. For example, the expression “FANA-cytidine” refers to a cytidine that comprises a 2′-fluoro-beta-D-arabinonucleic acid sugar modification. Within the context of this invention, the expression “a derivate thereof” refers to a corresponding nucleotide(s) or oligonucleotide(s) that has been chemically derived from said nucleotide or oligonucleotide(s).

As used herein, the term “complementary” or “partially complementary” or “substantially complementary” refer to nucleic acid sequences, which due to their complementary nucleotides are capable of specific intermolecular base-pairing. For example, the oligonucleotide may comprise a nucleic acid sequence complementary to a target sequence, e.g., SERPINA1 or any other target sequence. As those skilled in the art appreciate, in many instances, perfect complementary is not required and one or more wobbles (wobble base pairing), bulges, mismatches, etc. may be well tolerated. The one or more wobbles, bulges, mismatches may be within or outside the CBT. For example, the ASOs of the invention include a mismatch opposite the target adenosine. Hence, the complementarity of the ASOs of the invention may be 100%, except at the nucleoside opposite to a target nucleoside to be edited. In one embodiment, the complementarity is at least 80%, 85%, 90%, 95%. In one embodiment, the complementarity is 85%-99%. In another embodiment, the ASO comprises 1, 2, 3, 4 or 5 mismatches when aligned with the target nucleic acid. In one embodiment, the ASOs comprise a wobble base outside the CBT. In one embodiment, one or more mismatches are independently a wobble base paring. In one embodiment, the ASOs comprise up to 4 mismatches or wobble bases outside the CBT. In one embodiment, the ASOs comprise up to 3 mismatches or wobble bases outside the CBT.

The term “mutation” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Notably, the invention is not limited to correcting mutations, as it may instead be useful to change a wildtype sequence into a mutated sequence by applying the ASOs according to the invention. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)).

As used herein, the term “beneficial editing” refers to the editing of an RNA derived from a wildtype allele (not a mutated allele) in order to, e.g., modulate the function of a wildtype protein in a useful way to prevent or treat a disease. For example, beneficial editing may include sites, such as STAT1 Y701, NLRP3 Y166 and CTNNB1 T41 that are not causes for genetic diseases but rather represent wildtype protein sites. These sites are mutated (no underlying G-to-A mutation) to alter the function of the wildtype protein.

The term “compensatory editing” refers to the modification of RNA nucleotides to change and correct one or more detrimental or unfavourable changes in the RNA sequence when compared to wildtype, e.g., a compensatory A-to-I change could help to functionally compensate for an otherwise non-editable mutation to ameliorate a disease phenotype.

As used herein, the term “off-target” refers to nonspecific and unintended genetic modification(s) of the target. Specifically, off-target editing may include unintended point mutations, deletions, insertions, inversions, and translocations.

The term “adenosine deaminase(s)” or “adenosine deaminases acting on RNA” (ADARs), as used herein, refers to any (poly)peptide, protein or protein domain or fragment thereof capable of catalysing the hydrolytic deamination of adenosine to inosine. The term thus not only refers to full-length and wild type ADARs but also to a functional fragment or a functional variant of an ADAR. In some embodiments, the ADAR is an (endogenous) adenosine deaminase catalysing the deamination of adenosine to inosine or deoxy-adenosine to deoxyinosine. In some embodiments, the ADAR catalyses the deamination of adenine or adenosine in deoxyribonucleic acid (DNA) or in ribonucleic acid (RNA). The ADAR may be a human ADAR. The ADAR may be an endogenous ADAR. Accordingly, in some embodiments, the ADAR is an endogenous human ADAR1, ADAR2 or ADAR3 (hADAR1, hADAR2 or hADAR3), or any fragment or isoform(s) thereof (e.g., hADAR1 p110 and p150).

As used herein, the term “guide RNA” (gRNA) or “guide oligonucleotide” refers to a piece of RNA or oligonucleotide (comprising RNA and/or DNA) that functions as a guide for enzymes, with which it forms complexes. The guide RNA or guide oligonucleotide can comprise endogenous and/or exogenous sequences. Guides can be used in vitro and in vivo. For example, the guide RNA or guide oligonucleotide guides the base-modifying activity/editing function (e.g., ADAR) to the target to be edited in trans.

As used herein, the term “target RNA” typically refers to an RNA, which is subject to the editing reaction, and “targeted” by the respective ASOs of the invention.

As used herein, the terms “disease” or “disorder” are used interchangeably to refer to a condition in a subject. In certain embodiments, the condition is a disease in a subject, the severity of which is decreased by inducing an immune response in the subject through the administration of a pharmaceutical composition.

As used herein, the term “effective amount” in the context of administering a therapy to a subject refers to the amount of a therapy which has a prophylactic and/or therapeutic effect(s).

As used herein, the term “in combination” in the context of the administration of two or more therapies to a subject, refers to the use of more than one therapy (e.g., more than one prophylactic agent and/or therapeutic agent). The use of the term “in combination” does not restrict the order in which therapies are administered to a subject.

As used herein, the terms “prevent”, “preventing” and “prevention” in the context of the present invention and the administration of a therapy(ies) to a subject refers to the inhibition of the development or onset of a disease or a symptom thereof. In one embodiment, it relates to the administration of the compound to a patient who is known to have an increased risk of developing a certain condition, disorder, or disease.

As used herein, the terms “treat”, “treatment”, and “treating” refer in the context of the present invention to the administration of the compound to a patient, which has already developed signs and/or symptoms of a certain condition, disorder, or disease. Beneficial or desired clinical results include, but are not limited to, alleviation of symptoms; diminishment of the extent of a condition, disorder, or disease stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

The terms “subject” or “patient” are used interchangeable and relate to an animal (e.g., mammals) that may need administration of the compound of the invention in the field of human or veterinary medicine. In specific embodiments, the subject is a human. The subject may be administered the oligonucleotide of the invention for beneficial editing. The subject may be administered the oligonucleotide of the invention for compensatory editing.

As used herein, the term “pharmaceutically acceptable” means approved by a regulatory agency. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like. The formulation should suit the mode of administration.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used.

### (Antisense)Oligonucleotides

Provided herein are, inter alia, chemically modified (antisense)oligonucleotides (ASOs). While not intending to be bound by any particular theory of operation, it is believed that nucleobase and backbone linkage modifications of said ASOs are useful in stabilising and improving the editing efficacy and lysosomal stability of the oligonucleotides of the invention. Moreover, these modifications also have the potential to reduce the off-target editing of the different ASOs. Since the one or more modifications can be synthetically transferred to various oligonucleotide sequences, such modifications have the potential to improve the editing efficacy of oligonucleotides with different specificities. The ASOs of the invention can be used for several purposes. Advantageously, oligonucleotides provided herein might be useful in the editing of one or more G-to-A mutations. Notably, the ASOs of the invention are not just limited to correcting G-to-A mutations but are also useful in changing a wildtype sequence into a mutated sequence in order to modulate protein expression and/or function (“beneficialediting”) or to compensate for a mutation that is not a G-to-A mutation. Thus, the oligonucleotides and compositions comprising the same may be useful as active agents in medicine to treat genetic disorders, conditions or diseases associated with one or more G-to-A mutations.

The inventors of the present invention have realised that in the context of providing oligonucleotides for RNA editing, and to achieve a beneficial balance of high editing efficacy and lysosomal stability, it is necessary to incorporate certain features into the oligonucleotides. In particular, the inventors have found that oligonucleotides should have a mixture of different modifications at the 2′-position of the sugar residue, and that stretches of more than 6 nucleotides with the same 2′-modification should be avoided. Avoiding uniform blocks of more than 6 nucleotides with the same 2′-modification prevented a strong loss of editing activity with natural ADARs. In addition to this design feature, the modified oligonucleotides of the invention should have at least two of the three nucleotides of the CBT modified at the 2′-position of the sugar base or being deoxyribonucleosides, which permits added stabilization against nuclease digestion. In addition, it was found that phosphorothioate (PS) linkages should be avoided at positions h and i of the core sequence. PS linkages at such positions were found to impair editing strongly. The oligonucleotides of the invention do however benefit from having a base level of internucleoside linkage modifications elsewhere, as the inventors have found that having at least 15% modification is beneficial to achieve good RNA editing. It was beneficial for the oligonucleotides to incorporate modifications at the 2′-position of the nucleotides and that such modification should be composed of different groups. Accordingly, it was found that a mixture of 2′-F- and 2′-O-alkyl-modifications was beneficial, and that a minimum of 10% of each was desirable. When combined, these features conferred high levels of lysosomal stability and RNA editing efficacy of the oligonucleotides. Hence, the oligonucleotides of the invention are preferably modified and designed accordingly.

According to the invention, the core oligonucleotide comprises the sequence: 5′-N₋₅^{a}N₋₄^{b} N₋₃^{c}N₋₂^{d}N₋₁^{e}N₀^{f}N₊₁^{g}N₊₂^{h}N₊₃ⁱN₊₄^{j}-3′ and contain specific patterns of 2′-modification and internucleoside linkages, which contribute, inter alia, to the advantageous properties of the oligonucleotides. The core sequence may have, e.g., PS linkages at positions d, e and optionally a. Remarkably, the regions particularly sensitive to the discovered linkage pattern are the CBT and the adjacent hotspot (“extended hotspot”), where also very specific nucleotide modifications are required to adopt ideally to the ADAR's enzyme active site for editing efficacy. Generally, oligonucleotides containing a mixture of 2′-F-, 2′-OMe, and 2′-H modifications, at least 15% internucleoside linkage modifications, no more than 6 consecutive nucleotides of the same 2′-modification, and a 2′-O-alkyl-modification at the N₊₂ and a 2′-F-modification at N₊₃ provide (“extended hotspot”) optimal editing and lysosomal stability (e.g., Examples 15 for “extended hotspot”).

Accordingly, provided herein is a chemically modified oligonucleotide comprising a sequence with a length of 23 to 80 nucleotides, capable of binding to a target sequence in a target RNA, comprising a central base triplet (CBT) of 3 nucleotides (5′-N₋₁^{e}N₀^{f}N₊₁^{g}-3′) with the central nucleotide (N₀) directly opposite to the target adenosine in the target RNA, wherein the core oligonucleotide comprises the following sequence:
5′-N₋₅^{a}N₋₄^{b} N₋₃^{c}N₋₂^{d}N₋₁^{e}N₀^{f}N₊₁^{g}N₊₂^{h}N₊₃ⁱN₊₄^{j}-3′; and
wherein:
(a) at least two of the three nucleotides of the CBT are chemically modified at the 2′ position of the sugar moiety, are deoxyribonucleosides, or a combination thereof and wherein d and e are internucleoside linkage modifications;
(b) the N₊₂ nucleotide carries a 2′-O-alkyl-modification; and wherein the N₊₃ nucleotide carries a 2′-fluoro (2′-F)-modification;
(c) at least 10% of nucleotides are 2′-F-modified and at least 10% of nucleotides are 2′-O-alkyl-modified, wherein no more than 6 consecutive nucleotides have the same 2′-modification;
(d) the internucleoside linkage modification content is at least 15%; and
(e) linkages h and i are not phosphorothioate (PS) linkages.

Evidence of the advantageous effect of having a 2′-O-alkyl-modification at the +2 position and a 2′-fluoro (2′-F)-modification at the +3 position is provided by the inventors in the application (“modification hotspot”). Specifically, it is shown that constructs having a 2′-OMe at N₊₂ and a 2′-F at N₊₃ show high-level RNA editing yields. Hence, in one embodiment, the N₊₂ nucleotide is a 2′-O-alkyl-modification. In one embodiment, the N₊₃ nucleotide carries a 2′-F-modification. In one embodiment, the N₊₂ nucleotide is a 2′-O-alkyl-modification and the N₊₃ nucleotide is a 2′-F-modification.

The inventors have also shown that uniform blocks or a continuous stretch of nucleotides carrying the same chemical modification may interfere with the activity of the ASO. Hence, the oligonucleotides of the invention may be modified to not include uniform blocks or a continuous stretch of the same 2′-sugar modification. In one embodiment, no more than 6 consecutive nucleotides have the same 2′-modification. In one embodiment, no more than 5 consecutive nucleotides have the same modification. In one embodiment, no more than 4 consecutive nucleotides have the same modification. In one embodiment, no more than 3 consecutive nucleotides have the same modification. In one embodiment, no more than 2 consecutive nucleotides have the same modification.

Generally, metabolically unstable ASOs might be desirable for certain highly transient therapeutic effects, e.g., wound healing. Reversal of classical disease-causing point mutations require metabolically stable ASOs that reduce dosing frequency. While embodiments with low content of 2′-modification are well conceivable, the inventors aim at providing maximum stability by replacing each RNA nucleoside by either a 2′-modified RNA or DNA. As shown in the application, the inventors have realised that, provided blocks of no more than 6 consecutive nucleotides have the same 2′-modification, the oligonucleotides can tolerate high percentages of 2′-modifications without detrimental loss of activity. Accordingly, in one embodiment, 20-100%, 30-100%, 40-100%, 50-100%, 60-100%, 70-100%, 80-100%, or 90-100% of nucleotides are deoxyribonucleosides (DNA) or 2′-modified. In one embodiment, 20-100% of nucleotides are DNA or 2′-modified. In one embodiment, 50-100% of nucleotides are DNA or 2′-modified nucleotides. In one embodiment, 100% of nucleotides are DNA or 2′-modified nucleotides. In one embodiment, 30-95%, 40-95%, 40-90%, 50-95%, 50-90%, 60-95% or 60-90% of nucleotides are DNA or 2′-modified nucleotides. In one embodiment the above percentages are satisfied with only 2′-modified nucleotides and no DNA.

According to the invention, the oligonucleotides of the invention will comprise modifications at the 2′-position on nucleotides with different modifying groups being used. In one embodiment, 20-70% of nucleotides are 2′-F-modified. In one embodiment, 35-65% of nucleotides are 2′-F-modified. In one embodiment, 20-60% of nucleotides are 2′-O-methyl (2′-OMe)-modified. In one embodiment, 25-55% of nucleotides are 2′-OMe-modified.

The inventors have also realised that the modified oligonucleotides of the invention do not require all of the internucleoside linkages to be modified against lysosomal degradation, provided that a minimum level of internucleoside modification is incorporated and provided the linkages d and e of the core oligonucleotide sequence (as above) are modified. In one embodiment, the internucleoside linkage modification content is at least 15%. In one embodiment, the internucleoside linkage modification content is at least 20%, 25%, 30%, 35%, 40%, 45%, 50%, 55%, 60%, 65%, 70%, 80%, or 90%. In one embodiment, no more than 95%, 90%, 85%, 80%, 70%, 60%, 50%, 40%, or 30% of the linkages are internucleoside linkage modifications.

Internucleoside linkage modifications, such as PS linkages, tend to have a positive effect, interalia, on the pharmacokinetics as well as stability, protein binding, and intracellular localization of ASOs. However, at the same time, it is desirable to reduce overall PS content to reduce, e.g., toxicity and non-specific protein binding. In one embodiment, (a) no more than 95%, 90%, 85%, 80%, 70%, 60%, 50%, 40%, 30% or 20% of the linkages outside the CBT are internucleoside linkage modifications; or (b) 15-90% of the linkages are internucleoside linkage modifications, preferably wherein 40-80%, most preferably 45-60%, of the linkages are internucleoside linkage modifications. In one embodiment, no more than 95%, 90%, 85%, 80%, 70%, 60%, 50%, 40%, 30% or 20% of the linkages outside the CBT are internucleoside linkage modifications. In one embodiment, 15-90% of the linkages are internucleoside linkage modifications, preferably wherein 40-80%, most preferably 45-60%, of the linkages are internucleoside linkage modifications. In one embodiment, the internucleoside linkage modification content is at least 15%, 20%, 30%, 40%, 50%, 60%, 70%, 80% or 90%. In one embodiment, the internucleoside linkage modification content is no more than 95%, 90%, 85%, 80%, 75%, 70%, 65%, 60%, 55%, 50%, 45%, 40%, 35%, 30%, 25% or 20%. In one embodiment, the internucleoside linkage modification content is 10-90%, 15-90%, 15-80%, 15-70%, 15-60%, 20-90%, 10-80%, 20-80%, 25-80%, 30-80%, 30-90%, 40-90%, 40-80%, 40-70%, 45-90%, 45-85%, 45-75%, 45-70%, 45-60% or 45-55%. In one embodiment, 15-90% of the linkages are internucleoside linkage modifications. In one embodiment, 40-80% of the linkages are internucleoside linkage modifications. In one embodiment, 45-60% of the linkages are internucleoside linkage modifications. In one embodiment, the internucleoside linkage modification content is 20%, 30%, 40%, 45%, 50%, 60%, 70%, 80%, or 90%. In one embodiment, the internucleoside linkage modification content is 30%. In one embodiment, the internucleoside linkage modification content is 15%.

Oligonucleotides of different lengths may require a different mixture of particular 2′-modifications and internucleoside linkage modifications in order to provide optimal RNA editing. The shorter the oligonucleotide, the better might be the endosomal escape. Moreover, toxicity of the particular oligonucleotide may also depend on its length. Also, shorter oligonucleotides may experience higher specificity. On the other hand, while longer oligonucleotides may bind stronger or faster to their respective RNA target, editing-boosting bulges, mismatches and wobbles may also work better in long oligonucleotides. As a result, there is a benefit and/or trade-off for both long and short oligonucleotides of the invention. Accordingly, the oligonucleotides of the invention may be of varying lengths. The oligonucleotides may range from about 23-80 nucleotides in length, e.g., about 23-50 nucleotides in length or about 40-80 nucleotides in length. In one embodiment, the oligonucleotide has a length of 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. In a preferred embodiment, the oligonucleotide has a length of 59 nucleotides. In certain embodiments, the oligonucleotide has a length of 23 to 80 nucleotides. In some embodiments, the oligonucleotide has a length of 23-80, 23-70, 23-60, 23-50, 23-40, 23-33, or 23-38 nucleotides. In some embodiments, the oligonucleotide has a length of 25-80, 25-70, 25-60, 25-50, 25-40 nucleotides. In some embodiments, the oligonucleotide has a length of 30-80, 30-70, 30-60, 30-50, 30-40 nucleotides. In some embodiments, the oligonucleotide has a length of 40-80, 50-80, 60-80, or 70-80 nucleotides. In some embodiments, the oligonucleotide has a length of at least 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. In one embodiment, the oligonucleotide has a length of 28-60, 28-55, 28-50, 28-45, 28-40, 28-35, or 28-30 nucleotides. In one embodiment, the oligonucleotide has a length of 25-60, 25-55, 25-50, 25-45, 25-40, 25-35, or 25-30 nucleotides. In one embodiment, the oligonucleotide has a length of 28-70 nucleotides. In one embodiment, the oligonucleotide has a length of: (i) 28-60, 28-55, or 28-45 nucleotides; (ii) 59 nucleotides; or (iii) no more than 45 nucleotides. Ranges and lengths intermediate to the above recited ranges and lengths are also contemplated to be part of the invention. In one embodiment, the oligonucleotide has a length of 40 nucleotides. In one embodiment, the oligonucleotide has a length of 45 or less nucleotides and wherein outside of the CBT no more than 4 nucleotides are deoxynucleotides. In one embodiment, the oligonucleotide has a length of 40 nucleotides. In one embodiment, the oligonucleotide has a length of 45 nucleotides. In one embodiment, the oligonucleotide has a length of 35 nucleotides. In one embodiment, the oligonucleotide has a length of 33 nucleotides. In one embodiment, the oligonucleotide has a length of 32 nucleotides. In one embodiment, the oligonucleotide has a length of 30 nucleotides. In one embodiment, the oligonucleotide has a length of 25 nucleotides.

The inventors have found that longer oligonucleotides (around 40 to 80 nucleotides in length) comprising a mixture of modifications and designs as described above were able to tolerate a total deoxyribonucleoside content of 5-50%. Furthermore, the inventors surprisingly found that oligonucleotides comprising such modifications and designs could be further reduced to shorter sequences (≤45 nt) whilst still providing good RNA editing (e.g., Example 8 to 14). Moreover, shorter oligonucleotides containing no more than a total of 6 deoxyribonucleosides outside of the CBT and an internucleoside linkage modification content of at least 30% provided good RNA editing (e.g., Example 17). Continuous stretches and higher levels of internucleoside linkage modification content typically resulted in an increase in editing.

Accordingly, also provided herein is a modified oligonucleotide comprising a sequence with a length of 23 to 50 nucleotides, capable of binding to a target sequence in a target RNA, comprising a central base triplet (CBT) of 3 nucleotides (5′-N₋₁^{e}N₀^{f}N₊₁^{g}-3′) with the central nucleotide (N₀) directly opposite to the target adenosine in the target RNA, wherein the core oligonucleotide comprises the following sequence:
5′-N₋₅^{a}N₋₄^{b} N₋₃^{c}N₋₂^{d}N₋₁^{e}N₀^{f}N₊₁^{g}N₊₂^{h}N₊₃ⁱN₊₄^{j}-3′; and wherein:
(a) at least two of the three nucleotides of the CBT are chemically modified at the 2′ position of the sugar moiety, are deoxyribonucleosides, or a combination thereof and wherein d and e are internucleoside linkage modifications;
(b) the N₊₂ nucleotide carries a 2′-O-alkyl-modification; and wherein the N₊₃ nucleotide carries a 2′-fluoro (2′-F)-modification;
(c) at least 10% of nucleotides are 2′-F-modified and at least 10% of nucleotides are 2′-O-alkyl-modified, wherein no more than 6 consecutive nucleotides have the same 2′-modification;
(d) the regions 3′ and 5′ to the CBT do not contain more than a total of 6 deoxyribonucleosides;
(e) the internucleoside linkage modification content is at least 30%.

While the oligonucleotides of the invention tolerate DNA outside of the CBT, placement of a very high degree of DNA (or high DNA:RNA ratio) tends to interfere with editing efficiency. Nonetheless, potentially useful embodiments that contain a notable number of deoxyribonucleotides can be created that have a reasonable balance of stability versus editing efficiency. Hence, the oligonucleotides of the invention may contain different amounts of DNA. Specifically, the oligonucleotides may contain different amounts of DNA (2′-H modification) outside of the CBT. Thus, in one embodiment, the regions 3′ and 5′ to the CBT do not contain more than a total of 6 deoxyribonucleosides. In one embodiment, the regions 3′ and 5′ to the CBT do not contain more than a total of 5, 4, or 3 deoxyribonucleosides.

Outside of the CBT, 8, 7, 6, 5, 4, 3, 2, 1, or 0 nucleobases may be deoxyribonucleotides. In one embodiment, outside of the CBT, the oligonucleotide does not contain any deoxyribonucleosides. In one embodiment, outside of the CBT, no more than 1, 2, 3, or 4 nucleobases are deoxyribonucleotides. In one embodiment, outside of the CBT, no more than 3 nucleobases are deoxyribonucleotides. When compared to shorter ASOs, longer ASOs tend to tolerate higher amounts of DNA outside of the CBT. Hence, also provided herein is an oligonucleotide comprising a sequence with a length of 40 to 80 nucleotides, capable of binding to a target sequence in a target RNA, comprising a central base triplet (CBT) of 3 nucleotides (5′-N₋₁^{e}N₀^{f}N₊₁^{g}-3′) with the central nucleotide (N₀) directly opposite to the target adenosine in the target RNA, wherein the core oligonucleotide comprises the following sequence:
5′-N₋₅^{a}N₋₄^{b} N₋₃^{c}N₋₂^{d}N₋₁^{e}N₀^{f}N₊₁^{g}N₊₂^{h}N₊₃ⁱN₊₄^{j}-3′; and
wherein:
(a) at least two of the three nucleotides of the CBT are chemically modified at the 2′ position of the sugar moiety, are deoxyribonucleosides, or a combination thereof and wherein d and e are internucleoside linkage modifications;
(b) the N₊₂ nucleotide carries a 2′-O-alkyl-modification; and wherein the N₊₃ nucleotide carries a 2′-fluoro (2′-F)-modification;
(c) at least 10% of nucleotides are 2′-F-modified and at least 10% of nucleotides are 2′-O-alkyl-modified, wherein no more than 6 consecutive nucleotides have the same 2′-modification;
(d) the regions 3′ and 5′ to the CBT have a total deoxyribonucleoside content of 5-50%.

In one embodiment, the regions 3′ and 5′ to the CBT have a total deoxyribonucleoside content of 10-50%. In one embodiment, the deoxyribonucleoside content outside the CBT is 10-40%, more preferably 11-30%, and even more preferably 13-25%.

According to the invention, the chemically modified oligonucleotide contains internucleoside linkage modifications. In one embodiment, the oligonucleotide comprises at least one internucleoside linkage modification selected from the group consisting of phosphorothioate (PS), 3′-methylenephosphonate, 5-methylenephosphonate, 3′-phosphoroamidate, 2′-5′phosphodiester, and phosphoryl guanidine (PN). In a preferred embodiment, the internucleoside linkage modification is a PS linkage. In one embodiment, the internucleoside linkage modification is a 3′-methylenephosphonate linkage. In one embodiment, the internucleoside linkage modification is a 5′-methylenephosphonate linkage. In one embodiment, the internucleoside linkage modification is a 3′-phosphoroamidate linkage. In one embodiment, the internucleoside linkage modification is a 2′-5′-phosphodiester linkage. In one embodiment, the internucleoside linkage modification is a phosphoryl guanidine (PN) linkage. In one embodiment, the nucleic acid analogue is a PNA (peptide nucleic acid). In one embodiment, the nucleic acid analogue is PMO (phosphorodiamidate linked morpholino). In one embodiment the oligonucleotide comprises PS, phosphate (PO), and/or phosphorodiamidate linkages. In one embodiment, the at least one internucleoside linkage modification is PS. In one embodiment, the oligonucleotide contains a continuous stretch of PS linkages. In one embodiment, the continuous stretch of PS linkages is 10, 11, 12, 13, 14, 15, 16, 17, 18, 19, 20, 21, 22, 23, 24 or more linkages long.

The positioning of internucleoside linkages within the oligonucleotide plays an important role when determining a balance between high editing yields and a long half-life. At the same time, the need for a carefully considered placement of backbone modifications reflects the sensitivity of the CBT and bases surrounding the CBT for subtle chemical modifications on the ribose, the nucleobase or the linkage. For example, positions h and i may be chemically modified or unmodified. However, the inventors of the application discovered that PS linkages should not be placed at positions h and i, which are clearly detrimental to the editing efficacy of the constructs. Accordingly, in one embodiment, linkage h and i are not chemically modified. In one embodiment, linkage h is not chemically modified. In one embodiment, linkage i is not chemically modified. In some embodiments, linkages h and i are phosphate (PO) linkages. In some embodiments, linkages h and i are not phosphorothioate (PS) linkages. In one embodiment, up to three linkages selected from the group consisting of linkages b, c, f, g and j are also PS linkages. It is, however, excluded that all linkages a to j are PS linkages. In especially preferred embodiments the linkage f is a PS linkage. In especially preferred embodiments, linkages a, d and e are PS linkages whereas linkages h and i are PO linkages.

Stability and editing efficacy of the various oligonucleotides may be influenced by the amount and consecutive arrangement of the particular 2′-modifications. That is, repeated modifications of the same type of 2′-modification have been found to be detrimental to the RNA editing efficacy of the oligonucleotide. Hence, the inventors set out to investigate the effect of disrupting continuous stretches or blocks of identical 2′-modifications. As shown in the application, oligonucleotides comprising smaller blocks of no more than 6 consecutive nucleotides with the same 2′-modification provided the best editing efficacy. The oligonucleotides of the invention therefore do not contain uniform blocks of more than about 6 nucleotides with the same 2′-modification. For example, in one embodiment, the oligonucleotide comprises no more than 6 consecutive nucleotides that are 2′-F-modified and/or 2′-O-alkyl-modified. In one embodiment, no more than 4, 5, or 6 consecutive nucleotides are 2′-F-modified; and/or no more than 4, 5, or 6 consecutive nucleotides are 2′-O-alkyl-modified. In one embodiment, no more than 4, 5, or 6 consecutive nucleotides are 2′-F-modified. In one embodiment, no more than 4, 5, or 6 consecutive nucleotides are 2′-O-alkyl-modified. In one embodiment, the oligonucleotide contains 4 consecutive nucleotides that are 2′-F- and/or 2′-O-alkyl-modified. In one embodiment, the oligonucleotide contains 5 consecutive nucleotides that are 2′-F- and/or 2′-O-alkyl-modified. In one embodiment, the oligonucleotide contains 6 consecutive nucleotides that are 2′-F- and/or 2′-O-alkyl-modified. The oligonucleotides may contain fewer than 4 consecutive nucleotides with the same 2′-modification. In a preferred embodiment, the 2′-O-alkyl-modification is a 2′-OMe-modification.

Additionally, it was found that uniform and block-wise 2′-modification of the oligonucleotide as used in the prior art leads to a strong loss of editing activity. Without being bound by any theory, the inventors submit that this is in accordance with a negative effect of bulky 2′-modifications on the binding of dsRNA binding domains to dsRNA substrates. dsRNA recognition (e.g., by the deaminase and/or dsRBDs of ADAR) and dsRNA binding typically takes place through interactions of a protein with the minor groove of the RNA helix. 2′-ribose modifications project into the minor grove and create steric demands and change the hydrazination of the RNA helix. 2′-F modifications are sterically the most similar to the native 2′-OH in ribose but highly hydrophobic and may perturb the hydration. 2′-O-methyl-modifications are sterically more demanding and 2′-MOE-modifications even more so. Thus, it makes sense that bulky 2′-modifications are not well accepted, in particular not in large blocks as this would reject dsRBD binding. This is particularly true for 2′-MOE, but also for large blocks of 2′-O-methyl modifications. However, also continuous stretches of 2′-F are not ideal probably due to their strong hydrophobicity, but they are better accepted than 2′-O-methyl. Hence, mixing 2′-F and 2′-O-methyl modifications provides a means to create a duplex that provides easy binding access for ADAR. However, for the reasons given, 2′-F is better accepted.

Accordingly, the inventors discovered that avoiding uniform blocks of more than 6 nucleotides with the same 2′-modification prevented a strong loss of editing activity with natural ADARs. Thus, in one embodiment, less than 6, 5, 4, or 3 consecutive nucleotides have the same 2′-modification. In one embodiment, no more than 6 consecutive nucleotides are 2′-F-modified. In one embodiment, no more than 5 consecutive nucleotides are 2′-F-modified. In one embodiment, no more than 4 consecutive nucleotides are 2′-F-modified. In one embodiment, no more than 6 consecutive nucleotides are 2′-O-alkyl-modified. In one embodiment, no more than 5 consecutive nucleotides are 2′-O-alkyl-modified. In one embodiment, no more than 4 consecutive nucleotides are 2′-O-alkyl-modified, optionally wherein no more than 4 consecutive nucleotides are 2′-OMe-modified. In one embodiment, the oligonucleotide comprises 2, 3, 4, 5, or 6 consecutive nucleotides with the same 2′-modification, e.g., 5 consecutive nucleotides are 2′-F-modified.

The different kinds of PS-modification can impact the efficacy of the oligonucleotide. Hence, the instant application also provides chemically modified ASOs having reduced stereopure linkage chemistry, i.e., the ASOs of the invention relate to stereorandom PS-modified ASOs, which are generally easier and cheap in their production. Without being bound by any theory, the inventors submit that—contrary to the ASOs of the prior art—high levels of stereopure linkages may neither be useful nor necessary to provide efficient RNA editing. Given that the active site of the deaminase has to bind the target RNA/oligonucleotide drug duplex around the No position, certain linkages, e.g., where the protein interacts with the phosphate backbone, could potentially benefit from the insertion of stereopure linkage modifications, e.g., stereopure PS (or PN) modifications. However, the inventors believe that, if existing, these advantageous effects of stereopure linkage modifications may hold true only for a small number of sites; probably not more than 10 linkages per ASO, more likely for not more than 5 linkages per ASO. Hence, the inventors believe that a very high degree of stereopure linkages used in the prior art is not required to design ASOs with efficient editing.

Yet, introduction of (some) stereopure linkages may be used when applying an optimal 2′- and stereorandom linkage modification pattern in an oligonucleotide with an optimised length and asymmetry. Generally, the oligonucleotides comprise stereorandom internucleoside linkages. In one embodiment, the oligonucleotide comprises one or more stereorandom internucleoside linkage modifications. In one embodiment, the oligonucleotide does not comprise a stereopure PS linkage modification. In one embodiment, the oligonucleotide comprises no more than 10, preferably no more than 5 stereopure internucleoside linkages. In one embodiment, the oligonucleotide comprises no more than 5 stereopure internucleoside linkages. In one embodiment, the oligonucleotide comprises 1, 2, 3, 4, 5, 6, 7, 8, 9 or 10 stereopure internucleoside linkages.

While the oligonucleotides of the invention comprise different internucleoside linkage modifications, the inventors have shown that optimized PS linkage modifications in combination with balanced 2′-modification and reduced block sizes are beneficial in providing oligonucleotide with good RNA editing. Notably, without being bound by any theory, the inventors submit that different linkage modifications (or higher amounts of certain types, such as, e.g., phosphoryl guanidine (PN) linkages), may result in oligonucleotides that do not exhibit adequate RNA editing efficacy. Accordingly, in one embodiment, the stereopure linkages are PS linkages. In one embodiment, the stereopure linkages are PS linkages and/or PN linkages. In one embodiment, the stereopure linkages are PN linkages. In one embodiment, the oligonucleotide comprises no stereopure PS linkages and/or no stereopure PN linkages. In one embodiment, the oligonucleotide comprises no stereopure PS linkages. In one embodiment, the oligonucleotide comprises no stereopure PN linkages. In one embodiment, the chemically modified oligonucleotide does not comprise a stereopure PS linkage modification.

As previously mentioned, incorporation of higher levels of RNA into the oligonucleotide tends to make them metabolically unstable, which, however, can be attractive feature. To achieve the necessary metabolic stability of an oligonucleotide, the final oligonucleotide ideally should not contain any unmodified RNA nucleobases. In one embodiment, the oligonucleotide contains no unmodified RNA nucleobases. In one embodiment, the oligonucleotide contains more than 10%, 20%, 30%, 40%, 50%, 60%, 70%, 80%, or more than 90% modified nucleotides. In one embodiment, the oligonucleotide contains less than 90%, 80%, 70%, 60%, 50%, 40%, 30%, 20%, or less than 10% unmodified RNA nucleotides. In this context, the modifications include having deoxyribonucleotides.

2′-MOE residues are used for splice switching oligonucleotides and typically have very low toxicity. However, due to their bulkiness they are not well accepted in larger quantities. The inventors of the present invention have realized that while 2′-MOE modifications at the termini of the oligonucleotides did not affect overall editing yield, cellular toxicity of the test constructs was surprisingly reduced. Specifically, the inventors realised that the amount of 2′-MOE modifications could be limited to about no more than about 6, 7, or 8 nucleotides to still obtain good RNA editing. Similarly, the inventors realized that oligonucleotide comprising 2 to 6 LNAs provided good RNA editing.

Therefore, the oligonucleotide may comprise no more than 6, 7, or 8 2′-MOE modifications within the oligonucleotide. In one embodiment, the oligonucleotide comprises: (a) 2′-O-(2-methoxyethyl)-oligoribonucleotide (2′-MOE) terminal blocks at the 3′ and 5′ termini, wherein at each terminus there are no more than 4 nucleotides with 2′-MOE, preferably no more than 3 nucleotides with 2′-MOE; or (b) terminal locked nucleic acids (LNAs), wherein the oligonucleotide comprises 2 to 6 LNAs at each terminus or at the 5′ terminus, preferably wherein the oligonucleotide comprises 2 LNAs at each terminus or at the 5′ terminus. In one embodiment, at each terminus there are no more than 4 nucleotides with 2′-MOE, preferably no more than 3 nucleotides with 2′-MOE. In one embodiment, the oligonucleotide comprises 2′-MOE terminal blocks at the 3′ and 5′ termini, wherein at each terminus there are no more than 4 nucleotides with 2′-MOE. In one embodiment, the oligonucleotide comprises 2′-MOE terminal blocks at the 3′ and 5′ termini, wherein at each terminus there are no more than 3 nucleotides with 2′-MOE. In one embodiment, the oligonucleotide comprises terminal locked nucleic acids (LNAs), wherein the oligonucleotide comprises 2 to 6 LNAs at each terminus. In one embodiment, the oligonucleotide comprises terminal locked nucleic acids (LNAs), wherein the oligonucleotide comprises 2 to 6 LNAs at the 5′ terminus. In one embodiment, the oligonucleotide comprises 2 LNAs at each terminus. In one embodiment, the oligonucleotide comprises 2 LNAs at the 5′ terminus. In this context, the term “terminus” refers to the last or terminal nucleotides at either end of the oligonucleotide, e.g., “at each terminus there are no more than 4 nucleotides” refers to the last 4 nucleotides at each end of the oligonucleotide. In one embodiment, there is no 2′-MOE modification within the CBT. In one embodiment, there is no 2′-MOE modification within the +2 and/or +3 position.

In one embodiment, linkage g is not a PS linkage. In one embodiment, linkage g is a phosphate (PO) linkage.

In one embodiment, the 2′-O-alkyl-modification is a 2′-OMe-modification. Notably, in some cases, the 2′-O-alkyl-modification is not a 2′-MOE modification.

The CBT is very sensitive to position-specific linkage modification, which is due to interference with ADAR active site binding. Accordingly, to provide efficient editing and stabilisation of the oligonucleotide, the inventors have shown that (a mixture of) particular linkage modifications have to be placed at specific positions within the oligonucleotide. In one embodiment, d and e are PS linkage modifications, optionally wherein f is an internucleoside linkage modification. In one embodiment, d and e are PS linkage modifications. In one embodiment, f is a PS linkage.

In one embodiment, the modification at the 2′-position of the sugar moiety is a (i) 2′-O-alkyl-modification, (ii) 2′-F-modification, or (iii) 2′-fluoroarabinoside (FANA)-modification.

The CBT (5′-N₋₁-N₀-N₊₁-3′) may carry different modifications and permutations of the various modifications. In certain embodiments, the CBT is chemically modified. That is, positions N₋₁, N₀ and/or N₊₁ may carry modifications at the 2′ position. In one embodiment, only one position within the CBT is chemically modified. In one embodiment, two positions within the CBT are chemically modified. In one embodiment, all positions within the CBT are chemically modified. According to one embodiment, each of the three nucleosides of the CBT is either singularly or a combination of:
(a) a deoxyribonucleotide; and/or
(b) 2′-FANA-modification; and/or
(c) 2′-O-methyl-modification; and/or
(d) 2′-F-modification.

In one embodiment, at least one of the three oligonucleotides of the CBT is a deoxyribonucleotide. In one embodiment, at least one of the three oligonucleotides is 2′-FANA-modified. In one embodiment, at least one of the three oligonucleotides is —O-methyl-modified. In one embodiment, at least one of the three oligonucleotides is 2′-F-modified. In one embodiment,
(i) N₋₁ is 2′-F, 2′-FANA, DNA, or 2′-O-methyl; and/or
(ii) No is 2′-FANA or DNA; and/or
(iii) N+₁ is 2′-FANA, DNA, or 2′-O-methyl.
   In some embodiments, position N₋₁ is 2′-Fluoro-RNA, 2′-FANA or DNA. In some embodiments, position No is 2′-FANA or DNA. In some embodiments, position N₊₁ is 2′-FANA or DNA. CBT modification may comprise any permutation of the modifications described above.

In one embodiment, No is deoxycytidine or FANA-cytidine. In one embodiment, No is deoxycytidine. In one embodiment, No is FANA-cytidine. Other modifications may include nucleobase replacement by (N) heterocycles or aromatic rings that stack well in the RNA duplex, such as, e.g., a Benner's base Z (dZ) (and/or analogues) or 8-oxo-adenosine (8-oxo-A). Hence, in one embodiment, No is a Benner's base. In one embodiment, No is 8-oxo-adenosine.

The region and one or more nucleotides outside the CBT may be modified. For example, the nucleotides 5′ and/or 3′ to the CBT may be chemically modified to carry 2′-modifications. The oligonucleotides of the invention may be modified within an area that defines a “hotspot site” or “hotspot region”. In one embodiment, the oligonucleotide is modified at the first nucleotide directly 3′ to the CBT (i.e., position +2) (5′-^{d}N₋₁^{e}N₀^{f} N₊₁^{g} N₊₂^{h}N₊₃ⁱ-′3). In one embodiment, the oligonucleotide is modified at the second nucleotide directly 3′ to the CBT (i.e., position +3) (5′-^{d}N₋₁^{e}N₀^{f} N₊₁^{g}N₊₂^{h}N₊₃ⁱ-′3). In a preferred embodiment, the oligonucleotide is modified at the first two nucleotides directly 3′ to the CBT (i.e., positions +2 and +3) (5′-^{d}N₋₁^{e}N₀^{f} N₊₁^{g}N₊₂^{h}N₊₃ⁱ-′3).

The nucleotides 5′ and/or 3′ to the CBT may be chemically modified to carry 2′-modifications. In one embodiment, positions −5, −4, and −3 are 2′-O-alkyl-modified; and/or position −2 is 2′-F-modified. In one embodiment, positions −5, −4, and −3 are 2′-O-alkyl-modified. In one embodiment, position −2 is 2′-F-modified. In one embodiment, positions −5, −4, and −3 are 2′-O-alkyl-modified and position −2 is 2′-F-modified. In one embodiment, positions −5, −4, and −3 are 2′-O-alkyl-modified or position −2 is 2′-F-modified. In one embodiment, the 2′-OMe modification is at the +2 position. In one embodiment, the 2′-F modification is at the +2 position. In one embodiment, the 2′-F modification is at the −2 position. In one embodiment, the 2′-OMe modification is at the −5, −4, and −3 positions. In one embodiment, the 2′−OMe modification is at the −5, −4, or −3 positions. Other modifications may include nucleobase replacement by (N) heterocycles or aromatic rings that stack well in the RNA duplex, such as, e.g., a Benner's base Z (dZ) (and/or analogues) or 8-oxo-adenosine (8-oxo-A). In one embodiment, No is a Benner's base. In one embodiment, No is 8-oxo-adenosine (8-oxo-A). Specifically, in a preferred embodiment, the N₊₂ nucleotide is a 2′-O-alkyl-modification. In a preferred embodiment, the N₊₃ nucleotide carries a 2′-fluoro (2′-F)-modification. In the most preferred embodiment, the N₊₂ nucleotide is a 2′-O-alkyl-modification and the N₊₃ nucleotide is a 2′-fluoro (2′-F)-modification.

In one embodiment, 2′-OMe modifications are preferred over DNA close to the CBT. In one embodiment, 2′-F modifications are preferred over DNA close to the CBT. In one embodiment, there is no DNA at the +2 or +3 position.

As mentioned above, since internucleoside linkage modifications, such as PS linkages, tend to have a positive effect on, e.g., ASO stability, placement of these linkages within the ASO seems to play an important role. Amongst others, the inventors found that that PS linkages 3′ to DNA seem to be more important than 5′ to DNA in terms of tritosomal stability. Accordingly, in some embodiments, PS linkages are located 3′ to DNA. In one embodiment, PS linkages are located directly 3′ to DNA. In one embodiment, PS linkages are located 3′ and 5′ to DNA. In one embodiment there is only 2′-modified nucleotides within the ASO and no DNA. In one embodiment, the stability of the ASO is improved by placing PS linkages 3′ to DNA.

Uniform blocks or stretches of large 2′-sugar modifications within the ASO tend to interfere with the binding of ADAR's dsRNA binding proteins (dsRBDs). Hence, the oligonucleotides of the invention may be modified in a way to avoid such interference. For example, the oligonucleotides are modified such that they do not comprise continuous stretches or uniform blocks of nucleotides carrying the same chemical modification (i.e., avoidance of a block-like modification structure). Hence, in one embodiment, the oligonucleotide is not uniformly modified. In one embodiment, the oligonucleotide contains no uniform blocks and/or no block-like modification structure. In one embodiment, the oligonucleotide does not comprise continuous stretches or uniform blocks of nucleotides carrying the same chemical modification at the 2′ position of the sugar moiety. In a preferred embodiment, the oligonucleotides are modified as to avoid uniform blocks of 2′-F- and/or 2′-OMe-modifications. In some embodiments, the oligonucleotides do not contain any blocks of 2′-H (DNA). In a preferred embodiment, the oligonucleotides are modified as to avoid uniform blocks of 2′-F-modifications, 2′-OMe-modifications, and/or 2′-H groups. Notably, the maximum block size of 2′-F- and 2′-OME-modifications can differ. Accordingly, in one embodiment, the oligonucleotide comprises larger blocks of 2′F-modified nucleotides. In one embodiment, the oligonucleotide comprises larger blocks of 2′OMe-modified nucleotides. In one embodiment, 2′-OMe-modifications are accepted in smaller blocks than 2′-F-modifications. In one embodiment, 2′-F-modifications are accepted in larger blocks than 2′-OMe-modifications.

The oligonucleotide of the invention may contain some “continuous stretch(es)” or “uniform block(s)” of a certain length. In one embodiment, the size or length of the “continuous stretch(es)” or “uniform block(s)” is 2, 3, 4, 5, or 6 nucleotides long. In one embodiment, the size or length of the “continuous stretch(es)” or “uniform block(s)” is no more than 2, 3, 4, 5, or 6 nucleotides long. In one embodiment, the oligonucleotide comprises no more than 2, 3, 4, 5, or 6 consecutive nucleotides comprising a 2′-F modification. In one embodiment, the oligonucleotide comprises no more than 2, 3, 4, 5, or 6 consecutive nucleotides comprising a 2′-OMe modification. In one embodiment, one or more uniform blocks are interrupted. Interruption can take place by any other chemical modification (e.g., DNA, RNA, 2′-F, 2′-OMe, 2′-MOE, LNA, etc.). In one embodiment, one or more uniform blocks of 2′-F-modified nucleotides are interrupted, preferably by 2′-OMe-modified nucleotides. In one embodiment, one or more uniform blocks of 2′-OMe-modified nucleotides are interrupted, preferably by 2′-F-modified nucleotides. In some embodiments the blocks are disrupted by DNA.

According to the invention, the oligonucleotides do not contain blocks of more than 6 continuous 2′-OMe-modified nucleotides. According to the invention, the oligonucleotides do not contain blocks of more than 6 continuous 2′-F-modified nucleotides. In one embodiment, the oligonucleotides do not contain blocks of more than 5, 4, or 3 continuous 2′-OMe-modified nucleotides. In one embodiment, the oligonucleotides do not contain blocks of more than 4 continuous 2′-OMe-modified nucleotides.

Linkage g may be unmodified or modified. In one embodiment, linkage g is a phosphate (PO) linkage. In one embodiment, linkage g is a 3′,5′-phosphodiester linkage. In one embodiment, linkage g is a PS linkage. In one embodiment, the oligonucleotide comprises (a) at least 10 continuous internucleoside linkage modifications; and/or (b) 3 consecutive internucleoside linkage modifications at each terminus. In one embodiment, the oligonucleotide comprises at least 10 continuous internucleoside linkage modifications. In one embodiment, the oligonucleotide comprises 10, 11, 12, 13, 14, 15, 16, 17, 18, 19, 20, 21, 22, 23, 24, or more continuous PS linkages. In some embodiments, each terminus contains 3 consecutive internucleoside linkage modifications. In some embodiments, each terminus contains 4, 5, or 6 consecutive internucleoside linkage modifications. In some embodiments, each terminus contains no more than 8, 7, 6, 5, 4, or 3 consecutive internucleoside linkage modifications. In a preferred embodiment, each terminus contains 3 consecutive internucleoside linkage modifications. In some embodiments, the modification is a 3′-methylenephosphonate, 5-methylenephosphonate, 3′-phosphoroamidate, 2′-5′phosphodiester, or a phosphoryl guanidine (PN) modification. In one embodiment, the internucleoside linkage modification is a PS linkage modification. In another embodiment, the internucleoside linkage modification is a 3′-3′ or 5′-5′ phosphate ester bonds (3′-P-3′ and 5′-P-5′).

Without being bound by any theory, inventors submit that the ideal asymmetry for each target might depend on the length and the specific underlying sequence of the particular oligonucleotide. It is known that ADAR works as an asymmetric dimer with a footprint of up to 50 bp. While some substrates are more efficiently edited by the deaminase domain alone than by the full-length protein, the opposite holds true for other substrates. This suggests that depending on the size of the target/drug RNA helix, ADAR might bind in different ways (monomer versus dimer) and registers (with no, one, two or up to six dsRBDs). This leads to a situation, wherein, depending on the length of the ASO, specific symmetries on the target adenosine are and specific modifications patterns (e.g., ribose and linkage) are preferred. For an optimal binding of the deaminase, a short 3′ terminus seems to be sufficient (at least 4 nt beside the CBT). Elongation of the 3′ terminus may even lead to a loss of target editing. On the other hand, the 5′ terminus may provide binding space for the dsRBDs and thus typically requires more nucleotides (at least 16 nt). Some well-working embodiments of symmetries provided herein and identified by the inventors of the instant application are given in Table A.

Hence, in certain embodiment the ASO may be asymmetric. That is, there might be different numbers of nucleotides (nt) at the 3′ and 5′ end of the oligonucleotide. For example, there might be 20-40 nt at the 5′ terminus and 5-15 nt at the 3′ terminus. In one embodiment, there are
a) at least 4 nucleotides 3′ of the CBT; or
b) at least 16 nucleotides 5′ of the CBT.
   In some embodiments, there are 4-30 nt 3′ of the CBT. In some embodiments, there are no more than 10 nt 3′ of the CBT. In some embodiments, the 3′ terminus is shortened to a length of 5 nt 3′ of the CBT. In some embodiments, the 3′ terminus is shortened to a length of 4 nt 3′ of the CBT. In one embodiment, the region 3′ to the CBT contains 4, 5, or 6 nt. In some embodiments, there are 4-30 nt 5′ of the CBT. In one embodiment, there are no more than 35 nt 5′ of the CBT. In one embodiment, the 5′ terminus is shortened to a length of 25 nt or 26 nt 5′ of the CBT. In one embodiment, the region 5′ to the CBT contains 24, 25, or 26 nt.

According to the invention, the oligonucleotides may have a symmetry and length as disclosed in Table A below. The oligonucleotide has the following scheme: (length of 5′ terminus)-(CBT length)-(length of 3′terminus). For example, an ASO of the invention with a length of 32 nt, has a 5′ terminus that is 24 nt long, a CBT that is 3 nt long, and a 3′ terminus that is 5 nt long (Scheme “24-3-5”). Accordingly, in one embodiment, the oligonucleotide has any one of the symmetries listed in Table A.

TABLE A
Preferred asymmetries of some ASO
designs according to the invention.

| Exemplary Construct | | Scheme |
|---|---|---|
| Name | Length (nt) | (5′ terminus-CBT-3′ terminus) |
| SERPINA v117.142 | 32 | 24-3-5 |
| SERPINA v117.170 | | |
| CRB1 v117.42 | 32 | 24-3-5 |
| SERPINA v117.141 | 33 | 25-3-5 |
| STAT1 v117.56 | 33 | 25-3-5 |
| STAT1 v117.57 | 33 | 25-3-5 |
| CRB1 v117.44 | 38 | 28-3-7 |
| SERPINA v117.82 | 40 | 28-3-9 |
| SERPINA v117.123 | | |
| SERPINA v117.158 | | |
| CRB1 v117.30 | 45 | 33-3-9 |
| CRB1 v117.31 | | |

Depending on the length and/or minimal degree in linkage modification of a particular oligonucleotide, it may be differentially modified as described within this application. It is known that DNA prefers a different sugar puckering than RNA and that 2′-modified RNA leads to a preferred B-form helix. Moreover, DNA is fairly hydrophobic and changes the hydrazination of the double helix. Thus, DNA is only accepted at certain positions and is not well accepted in larger blocks. Accordingly, the oligonucleotides provided herein may have different ratios and amounts of DNA and/or RNA. In some embodiments, the oligonucleotides have a combination of RNA and DNA. In some embodiments, the oligonucleotides have a combination of RNA and DNA outside of the CBT.

Moreover, the oligonucleotides of the invention may have a limited DNA content outside of the CBT. In one embodiment, DNA is located outside the CBT. In one embodiment, DNA is located 3′ and/or 5′ of the CBT. In one embodiment, DNA is located 3′ of the CBT. In one embodiment, DNA is located 5′ of the CBT. Shorter oligonucleotides (≤45 nt) may have a lower DNA content than longer oligonucleotides (50 nt). In one embodiment, the oligonucleotide has a length of 45 nt. In one embodiment, the oligonucleotide has a length of 45 nt or less nucleotides and no more than 3 nucleotides outside of the CBT are deoxynucleotides. In one embodiment, no more than 1, 2, 3 or 4 nucleotides outside of the CBT are deoxyribonucleotides. In one embodiment, the nucleotide has a length of 28-60, 28-55, or 28-45 nucleotides. In one embodiment, the nucleotide has a length of 28-60 and a deoxyribonucleoside content outside the CBT that is 10-40%, more preferably 11-30%, and even more preferably 13-25%. In one embodiment, the oligonucleotide does not contain any unmodified RNA nucleotides.

In one embodiment, at least one of the three nucleotides of the CBT is chemically modified at the 2′ position of the sugar moiety, wherein said modification is a 2′-F-modification.

Loop-hairpin structured oligonucleotides have previously been described (WO 2020/001793) and used successfully to harness ADARs with chemically modified oligonucleotides. However, they are comparably large and—without being bound by any theory—the inventors believe that a more intelligent design of the ASO can form a substrate duplex that is also very well and quickly recognized by endogenous ADAR so that the large recruitment motifs can be omitted. For the delivery and manufacture this is a clear advantage as much shorter ASOs can be designed. Hence, the oligonucleotides may or may not include a recruitment motif for a deaminase. Instead, the chemically modified nucleic acids of the present invention form an RNA duplex to which the ADAR enzyme adheres, whereby the editing efficiency is increased. In one embodiment, the oligonucleotide does not comprise a loop-hairpin structured ADAR recruitment motif.

In certain cases, the ASO targeting domain, or nucleobase opposite to the target nucleobase that is to be edited, comprises, one or more wobble bases to compensate for the variability in the target sequence. That is, the less stringent base-pairing requirement of the wobble base (e.g., G-U, I-A, G-A, I-U, I-C, etc.) allows the ASO to pair with more than just one target nucleic acid. Accordingly, in some embodiments, mismatches and/or wobbles enable targeting of different target nucleic acids. In one embodiment, the oligonucleotide comprises one or more additional mismatches, wobble base and/or bulges. In some embodiments, the oligonucleotides of the invention may contain bulges of 1, 2, 3 or more nucleotides. In one embodiment, the oligonucleotide comprises one or more mismatches, wobble base, and/or bulges with respect to its target, and/or a mismatch at No. In one embodiment, the oligonucleotide comprises one or more mismatches, wobble base, and/or bulges with respect to its target. In one embodiment, the oligonucleotide comprises a mismatch at No.

The targeting sequence of the artificial nucleic acid typically comprises a nucleic acid sequence complementary or at least partially complementary to a nucleic acid sequence in the target RNA. In some embodiments, the targeting sequence comprises a nucleic acid sequence complementary or at least 60%, 70%, 80%, 90%, 95% or 99% of a nucleic acid sequence in the target RNA.

While the oligonucleotides may comprise DNA and/or RNA, they may also comprise additional modifications. LNAs improve the binding power of ASOs by preserving the nucleoside in a preferred sugar confirmation (entropic favour). However, this preorganisation of the sugar by the additional bridge also reduces flexibility. Double-stranded RNA (dsRNA) structures are strongly perturbed in the active site of ADAR (flip-out mechanism). LNA may interfere with this process and thus it is desirable to place any LNAs in positions that are not inside or too close to the CBT. In one embodiment, the oligonucleotide comprises LNA(s). In one embodiment, the oligonucleotide comprises DNA and/or RNA and/or LNA. In one embodiment, the oligonucleotide comprises DNA, RNA and LNA. In one embodiment, the oligonucleotide comprises DNA. In one embodiment, the oligonucleotide comprises RNA. In one embodiment, the oligonucleotide comprises LNA(s).

In addition to the specific backbone linkage modification pattern and modifications at the 2′-position of the sugar moiety, the purines and/or pyrimidines of the oligonucleotide may be specifically targeted. Purines and/or pyrimidines may be modified or unmodified. In one embodiment, purines and/or pyrimidines are modified. In some embodiments, a nucleobase is a substituted purine base residue. In some embodiments, a nucleobase is a substituted pyrimidine base residue. In one embodiment, purines are modified with 2′-OMe, 2′-F, or 2′-deoxy. In one embodiment, pyrimidines are modified with 2′-OMe, 2′-F, or 2′-deoxy. In some embodiments, the nucleobase is a substituted heterocyclic base analogue. In some embodiments, the heterocyclic base analogue is a nitrogen (N), oxygen (O), sulfur (S), or boron (B) heterocyclic base analogue. In certain embodiments, the modification includes a Benner's base Z and/or analogues thereof.

Oligonucleotides of the invention may be modified at their 5′ and/or 3′ termini. The oligonucleotides of the invention may comprise one or more different linkers, tags or coupling agents at either one or both termini. For example, the oligonucleotides may comprise amino-linkers, preferably C6-amino-linkers. Hence, in some embodiments, the oligonucleotides of the invention comprise a C6-amino-linker at the 5′ terminus. In some embodiments, the oligonucleotides comprise a C6-amino-linker at the 3′ terminus. The oligonucleotides may comprise a moiety, which enhances cellular uptake of the oligonucleotide, e.g., N-acetylgalactosamine (GalNAc). Hence, in some embodiments, the chemically modified oligonucleotide comprises a moiety or is conjugated to a moiety that enhances cellular uptake of the oligonucleotide. Preferably, the moiety enhancing cellular uptake is a triantennary N-acetyl galactosamine (GalNAc3), which is preferably conjugated to the 3′ terminus or to the 5′ terminus of the oligonucleotide.

### Compositions

The nucleic acids or oligonucleotides (orASOs) provided herein may be incorporated into compositions. For instance, targeted delivery of oligonucleotides to liver hepatocytes using bi- or triantennery N-acetylgalactosamine (GalNAc) conjugates has previously described for, e.g., treating liver diseases, including Hepatitis B virus (HBV), non-alcoholic Fatty Liver Disease and genetic diseases (Debacker et al., 2020).

Accordingly, provided herein is a composition containing the oligonucleotide(s) of the invention. In some embodiments, the present disclosure provides oligonucleotide compositions of oligonucleotides described herein. In some embodiments, the compositions are pharmaceutical compositions. As used herein, pharmaceutical composition means a mixture of substances suitable for administering to an individual. For example, a pharmaceutical composition may comprise one or more active pharmaceutical agents (such as an oligonucleotide) and a sterile aqueous solution. In one embodiment, the composition contains one or more oligonucleotides of the invention.

The pharmaceutical compositions provided herein can be in any form that allows for the composition to be administered to a subject. The compositions may be used in methods of treating and/or preventing a genetic disorder, condition, or disease. In a specific embodiment, the pharmaceutical compositions are suitable for veterinary and/or human administration.

Provided herein is a pharmaceutical composition comprising the oligonucleotide of the invention or a pharmaceutically acceptable salt thereof.

In one embodiment, a composition comprises an oligonucleotide of the invention in an admixture with a pharmaceutically acceptable carrier. In some embodiments, the pharmaceutically acceptable carrier can simply be a saline solution. This can be isotonic or hypotonic.

In some embodiments, a pharmaceutical composition may comprise one or more other therapies in addition to an oligonucleotide of the invention.

In certain embodiments, the compositions of the invention further include diluents of various buffer content (e.g., Tris-HCl, acetate, phosphate), pH, and ionic strength, and additives such as detergents and solubilizing agents (e.g., Tween 80, Polysorbate 80), anti-oxidants (e.g., ascorbic acid, sodium metabisulfite), preservatives (e.g., Thimersol, benzyl alcohol), and bulking substances (e.g., lactose, mannitol). In some embodiments, the material may be incorporated into particulate preparations of polymeric compounds such as polylactic acid, polyglycolic acid, etc. or into liposomes. In some embodiments, hyaluronic acid may also be used. Such compositions may influence the physical state, stability, rate of in vivo release, and/or rate of in vivo clearance of the present ASOs and derivatives. In some embodiments, the compositions may be prepared in liquid form, or may be in dried powder, such as lyophilized form.

In certain embodiments, the pharmaceutical compositions described herein additionally comprise one or more salts, e.g., sodium chloride, calcium chloride, sodium phosphate, monosodium glutamate, and aluminium salts (e.g., aluminium hydroxide, aluminium phosphate, alum (potassium aluminium sulfate), or a mixture of such aluminium salts). In other embodiments, the pharmaceutical compositions described herein do not comprise salts.

The pharmaceutical compositions described herein can be included in a container, pack, or dispenser together with instructions for administration.

### Prophylactic and Therapeutic Uses

The invention generally describes the use of the chemically modified oligonucleotide and/or composition comprising the same in the medical setting. Specifically, for site-directed editing of a target RNA (e.g., binding to the target RNA via the targeting sequence and by recruiting to the target site a deaminase). The present invention describes chemically modified oligonucleotide and/or composition for use in the treatment or prevention of a genetic disorder, condition, or disease as well as methods for treating or preventing a genetic disorder, condition, or disease. Site-directed editing may take place in vitro, in vivo or ex vivo.

A chemically modified oligonucleotide or composition comprising the same may be used in the treatment and/or prevention of a medical condition. In one aspect provided herein is the use of an oligonucleotide of the invention and/or a composition comprising the same in the treatment or prevention of a genetic disorder, condition, or disease. In one embodiment, the genetic disorder, condition or disease is selected from the group consisting of: Retinitis pigmentosa (RP), Stargardt macular degeneration, age-related macular degeneration (AMD), Choroideremia, Cone-rod Dystrophy, Cystic fibrosis (CF), Hurler Syndrome, alpha-1-antitrypsin (A1AT) deficiency, Parkinson's disease, Alzheimer's disease, albinism, Amyotrophic lateral sclerosis, Asthma, β-thalassemia, Cadasil syndrome, Charcot-Marie Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis (LCA), Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders.

In one embodiment, the genetic disorder, condition, or disease is associated with a point mutation. For example, the SERPINA1 gene encodes serine protease inhibitor alpha-I antitrypsin (A1AT). A1AT protects tissues from certain inflammatory enzymes, including neutrophil elastase. A deficiency in A1AT (alpha 1 antitrypsin deficiency, A1AD) can lead to excessive break down of elastin in the lungs by neutrophil elastase. This may lead to reduced elasticity in the lungs and subsequent respiratory complications, including emphysema and chronic obstructive lung disease (COPD). Mutant A1AT can also build up in the liver, resulting in cirrhosis and liver failure. Accordingly, in one embodiment, the genetic disorder, condition or disease is associated with a G-to-A mutation in genes selected from the list comprising: SERPINA1, PDE6A, LRRK2, and CRB1. In one embodiment, the mutation is selected from the list comprising: SERPINA1 E342K, PDE6A V685M, NLRP3 Y166, and CRB1 C948Y In one embodiment, the mutation is the PiZZ mutation (α1-antitrypsin deficiency).

A chemically modified oligonucleotide of the invention or composition comprising the same may be used to edit adenosine bases in wildtype alleles (beneficial editing). In one embodiment, such editing modulates signalling, e.g., JAK/STAT signalling. In one embodiment, editing introduces a STAT1 Y701C change. In one embodiment, editing modulates inflammasome signalling by introducing a NLRP3 Y166 to C mutation.

The chemically modified oligonucleotide of the invention or the (pharmaceutical) composition may be administered, for example, orally in any orally acceptable dosage form including, but not limited to, capsules, tablets, aqueous suspensions, or solutions, or parenterally, e.g., by parenteral injection. In some embodiments, formulations suitable for parenteral administration comprise sterile aqueous preparations of at least one embodiment of the present disclosure, which are approximately isotonic with the blood of the intended recipient. The amount of oligonucleotide or composition to be administered, the dosage and the dosing regimen can vary from cell type to cell type, the disease to be treated, the target population, the mode of administration (e.g., systemic versus local), the severity of disease and the acceptable level of side activity. In some embodiments, the amount of oligonucleotides administered in a pharmaceutical composition is dependent on the subject being treated, the subject's weight, the manner of administration.

Various delivery systems can be used to deliver the oligonucleotides of the invention. An oligonucleotide according to the invention can be delivered as is (i.e., naked and/or in isolated form) to an individual, an organ (the eye), or specifically to a cell. When administering an oligonucleotide according to the invention, it is preferred that the oligonucleotide is dissolved in a solution that is compatible with the delivery method. Such delivery may be in vivo, in vitro or ex vivo. Nanoparticles and micro-particles that may be used for in vivo ASO delivery are well known in the art. Alternatively, a plasmid can be provided by transfection using known transfection reagents.

In a preferred embodiment, the oligonucleotides of the present invention are administered and delivered ‘as is’, also referred to as ‘naked’. Nevertheless, the art contains multiple ways of delivering oligonucleotides to cells, either in vitro, ex vivo or in vivo. That is, depending on the disease, disorder or infection that needs to be treated, or on the cell, tissue or part of the body that needs to be reached by the oligonucleotides of the present invention (e.g., in case of beneficial editing), an administration route or delivery method may be selected. Examples for delivery when an oligonucleotide is not delivered naked, are delivery agents or vehicles such as nanoparticles, like polymeric nanoparticles, liposomes, antibody-conjugated liposomes, cationic lipids, polymers, or cell-penetrating peptides.

Use of an excipient or transfection reagents may aid in delivery of each of the oligonucleotides or compositions as defined herein to a cell and/or into a cell (preferably a cell affected by a G-to-A mutation or that wherein “beneficial editing” is to be achieved as outlined herein). Preferred are excipients or transfection reagents capable of forming complexes, nanoparticles, micelles, vesicles and/or liposomes that deliver each oligonucleotide or composition as defined herein, complexed or trapped in a vesicle or liposome through a cell membrane. Many of these excipients are known in the art. Suitable excipients or transfection reagents comprise polyethylenimine (PEI; ExGen500 (MBI Fermentas)), LipofectAMINE™ 2000 (Invitrogen), Lipofectin™, or derivatives thereof, and/or viral capsid proteins that are capable of self-assembly into particles that can deliver each constituent as defined herein to a target cell. Such excipients have been shown to efficiently deliver oligonucleotides to a wide variety of cultured cells. Their high transfection potential is combined with an excepted low to moderate toxicity in terms of overall cell survival.

An ASO of the present invention can be linked to a moiety that enhances uptake of the ASO in cells. Examples of such moieties are cholesterols, carbohydrates, vitamins, biotin, lipids, phospholipids, cell-penetrating peptides including but not limited to antennapedia, TAT, transportan and positively charged amino acids such as oligoarginine, poly-arginine, oligolysine or polylysine, antigen-binding domains such as provided by an antibody, a Fab fragment, or a single chain antigen binding domain such as a cameloid single domain antigen-binding domain. Accordingly in some embodiments, the ASO is delivered using drug conjugates with antibodies, nanobodies, cell penetrating peptides and aptamers. In one embodiment, the oligonucleotide is conjugated to an antibody, preferably a Fab fragment.

In some embodiments, toxicity and therapeutic efficacy can be determined by standard pharmaceutical procedures in cell cultures or experimental animals, e.g., for determining the LD₅₀ (the dose lethal to 50% of the population) and the LD₅₀ (the dose therapeutically effective in 50% of the population). In some embodiments, data obtained from the cell culture assays or animal studies can be used in formulating a range of dosage for use in humans.

The oligonucleotide or composition may be administered as a monotherapy or in combination with a further different medicament, particularly a medicament suitable for the treatment or prevention Retinitis pigmentosa (RP), Stargardt macular degeneration, age-related macular degeneration (AMD), Cystic fibrosis (CF), Hurler Syndrome, alpha-1-antitrypsin (A1AT) deficiency, Parkinson's disease, Alzheimer's disease, albinism, Amyotrophic lateral sclerosis, Asthma, β-thalassemia, Cadasil syndrome, Charcot-Marie Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders and/or symptoms associated therewith. In a preferred embodiment, the oligonucleotide or composition may be administered as a monotherapy or in combination with a further different medicament for the treatment of any retinal disease, including, e.g., inherited retinal diseases such as retinitis pigmentosa (RP), Choroideremia, Stargardt Disease, cone-rod dystrophy and/or Leber Congenital Amaurosis (LCA).

In some embodiments, compared to reference oligonucleotides or compositions, the provided oligonucleotides or compositions are surprisingly effective. In some embodiments, a change is measured by an increase of a desired mRNA and/or protein level compared to a reference condition. In some embodiments, a change is measured by an increase or decrease in editing efficacy mediated by the oligonucleotide or composition comprising the same. In some embodiments, a change is measured by an increase in stability of the oligonucleotide or composition comprising the same.

Further provided herein is a method of targeting adenosines. Specifically, provided herein is a method of targeting wildtype adenosines for beneficial and/or compensatory RNA editing. Provided herein is a method for targeting wildtype adenosines for beneficial editing. Provided herein is a method for targeting wildtype adenosines for compensatory editing.

Also provided herein is a method of treating a subject suffering from a genetic disorder, condition, or disease, wherein the method comprises administering to the subject in need thereof an effective amount of the chemically modified oligonucleotide of the invention or composition of the invention. In one embodiment, the genetic disorder, condition, or disease is associated with a G-to-A mutation. Treating disorders associated with G-to-A mutations can result in a decrease in the mortality rate of a population of treated subjects in comparison to an untreated population.

Provided herein is the use of an oligonucleotide of the invention in therapy. Also, provided herein is the use of an oligonucleotide of the invention in the manufacture of a medicament for treating a condition, disorder or disease associated with a G-to-A mutation. Also provided herein is the use of an oligonucleotide of the invention in the manufacture of a medicament for treating a genetic disorder, condition or disease associated with a G-to-A mutation. Also provided herein is a use of an oligonucleotide of the invention in the manufacture of a medicament for treating a genetic disorder, condition or disease associated with a G-to-A mutation.

The compositions of the present disclosure can be administered in a number of ways depending upon whether local or systemic treatment is desired and upon the area to be treated. Administration may be by inhalation (e.g., through nebulization), intranasally, orally, by injection or infusion, intravenously, subcutaneously, intra-dermally, intra-cranially, intramuscularly, intra-tracheally, intra-peritoneally, intra-rectally, by direct injection into a tumour, and the like. Administration may be in solid form, in the form of a powder, a pill, or in any other form compatible with pharmaceutical use in humans. In some embodiments the oligonucleotide construct can be delivered systemically.

### Patient Population

The oligonucleotides of the invention of compositions comprising the same may be administered to various groups of subjects or patients. In certain embodiments, the patient is in need of treatment. In other embodiments, the patient is not in need of treatment (“beneficial editing”), that is, the subject receives the oligonucleotide or composition to edit an RNA derived from a wildtype allele (not a mutated allele) in order to, e.g., modulate the function of a wildtype protein in a useful way to prevent or treat a disease.

In certain embodiments, an oligonucleotide or composition containing an oligonucleotide described herein is be administered to a naive subject, i.e., a subject that does not have a disease or disorder. In one embodiment, an oligonucleotide or composition containing an oligonucleotide described herein is be administered to a subject. In one embodiment, an oligonucleotide or composition provided herein is administered to a naive subject that is at risk of developing a disease or disorder.

In certain embodiments, an oligonucleotide or composition containing an oligonucleotide described herein is administered to a patient who has been diagnosed with a disease or disorder. In some embodiments, an oligonucleotide or composition containing an oligonucleotide described herein described herein is administered to a patient before symptoms manifest or symptoms become severe.

In some embodiments, an oligonucleotide or composition containing an oligonucleotide described herein is administered to a human. In some embodiments, the human subject to be administered an oligonucleotide or composition containing an oligonucleotide described herein is any individual at risk of developing a disease or disorder associated with a G-to-A mutation in genes. In one embodiment, the patient suffers from a disease or disorder associated with a G-to-A mutation in genes.

In some embodiments, a subject or patient suitable for treatment of a condition, disorder, or disease associated with a G-to-A mutation, can be identified or diagnosed by a health care professional. In some embodiments, a symptom of a condition, disorder or disease associated with a G-to-A mutation can be any condition, disorder or disease that can benefit from an A-to-I conversion.

Also provided herein are methods of treating a condition, disorder or disease associated with a G-to-A mutation in a subject. In some embodiments, a method of the present disclosure can be for the treatment of a condition, disorder or disease associated with a G-to-A mutation in a subject wherein the method comprises administering to a subject a therapeutically effective amount of an oligonucleotide or a pharmaceutical composition thereof.

Also provided herein is a use of an oligonucleotide of the invention in therapy. Also, provided herein is a use of an oligonucleotide of the invention in the manufacture of a medicament for treating conditions, diseases and/or disorders associated with a G-to-A mutation in a subject. Also provided herein is a use of an oligonucleotide of the invention in the manufacture of a medicament for treating a condition, disease and/or disorder associated with a G-to-A mutation. In certain embodiments, the use of an oligonucleotide of the invention is in the manufacture of a medicament for treating a condition, disease and/or disorder associated with a G-to-A mutation.

The composition of the invention comprises the oligonucleotide of the invention. According to a further aspect, the invention relates to a kit or kit of parts comprising an oligonucleotide of the invention and/or the (pharmaceutical) composition according to the invention. The kit additionally comprises instructions for use.

### Methods for Editing

The present invention also relates to methods for editing a target adenosine in a target nucleic acid. For example, the present invention provides methods of editing a SERPINA1 polynucleotide, e.g., a SERPINA1 polynucleotide comprising a single nucleotide polymorphism (SNP) associated with alpha I antitrypsin deficiency. Moreover, the present invention relates to in vitro methods for editing a target adenosine in a target nucleic acid and in vitro method for deaminating at least one specific adenosine present in a target RNA sequence in a cell.

In one aspect provided herein is an in vitro method for editing a target adenosine in a target nucleic acid, wherein the method comprises contacting the target nucleic acid with the oligonucleotide of the invention.

In another aspect provided herein is an in vitro method for deaminating at least one specific adenosine present in a target RNA sequence in a cell, wherein the method comprises the steps of:
(a) contacting the target nucleic acid with a chemically modified oligonucleotide of the invention;
(b) allowing uptake by the cell of the chemically modified oligonucleotide;
(c) allowing annealing of the chemically modified oligonucleotide to the target RNA sequence; and
(d) allowing a mammalian ADAR enzyme comprising a natural dsRNA binding domain as found in the wild type enzyme to deaminate the target adenosine in the target RNA sequence to an inosine.

In one embodiment, the method comprises after step (d), a step of identifying the presence of the inosine in the RNA sequence.

The editing reaction is preferably monitored or controlled by sequence analysis of the target RNA.

Also, a chemically modified oligonucleotide of the invention or a (pharmaceutical) composition may be used in the diagnosis of a genetic condition, disease, or disorder. Therein, the disease or disorder is preferably selected from the group consisting of infectious diseases, tumour diseases, cardiovascular diseases, autoimmune diseases, allergies and neurological diseases or disorders. In one embodiment, the genetic disorder, condition, or disease is associated with a G-to-A mutation.

The invention is used to make desired changes in a target sequence in a cell or a subject by site-directed editing of nucleotides using an oligonucleotide that is capable of effecting an adenosine deaminase acting on RNA (ADAR)-mediated adenosine to inosine. As a result, the target sequence is edited through an adenosine deamination reaction mediated by ADAR, converting adenosines into inosine. In some embodiments, because I is recognized as G, the deamination correcting the pathogenic mutation in the SERPINA1 gene reverses the E342K mutation back to wild-type, reversing or slowing symptoms associated with A1AD experienced by the patient.

The methods of the present invention can be used with cells from any organ, e.g., skin, lung, heart, kidney, liver, pancreas, gut, muscle, gland, eye, brain, blood and the like. The invention is particularly suitable for modifying sequences in cells, tissues or organs implicated in a diseased state of a (human) subject. For example, such cells may include, but are not limited, to hepatocytes, hepatocyte like cells, and/or alveolar type II cells, neurons (PNS, CNS), retina, photo receptors cells, Müller Glia cells, RPE, immune cells, B cells, T cells, dendritic cells, macrophages.

The present invention shall be described in more detail by the following Figures and Examples.

### EXAMPLES

The examples shown in the following are merely illustrative and shall describe the present invention in a further way. These examples shall not be construed to limit the present invention thereto. The sequences disclosed herein are also shown in the enclosed sequence listing. However, the sequence listing shows only the sequence of nucleotides, whereas the modification of the nucleotides and of the bonds between the nucleotides is not shown in the sequence listing. The relevant modifications associated with the sequences are disclosed in the tables below and, to some extent, in the Figures of this application.

For all experiment, editing efficacy is expressed as the percentage [%] of edited target sites found in all detected target sites in the target transcript.

### Example 1. Editing Efficacy and Lysosomal Stability of SERPINA1 E342K Targeting Oligonucleotides with Increasing DNA to RNA (DNA:RNA) Ratios

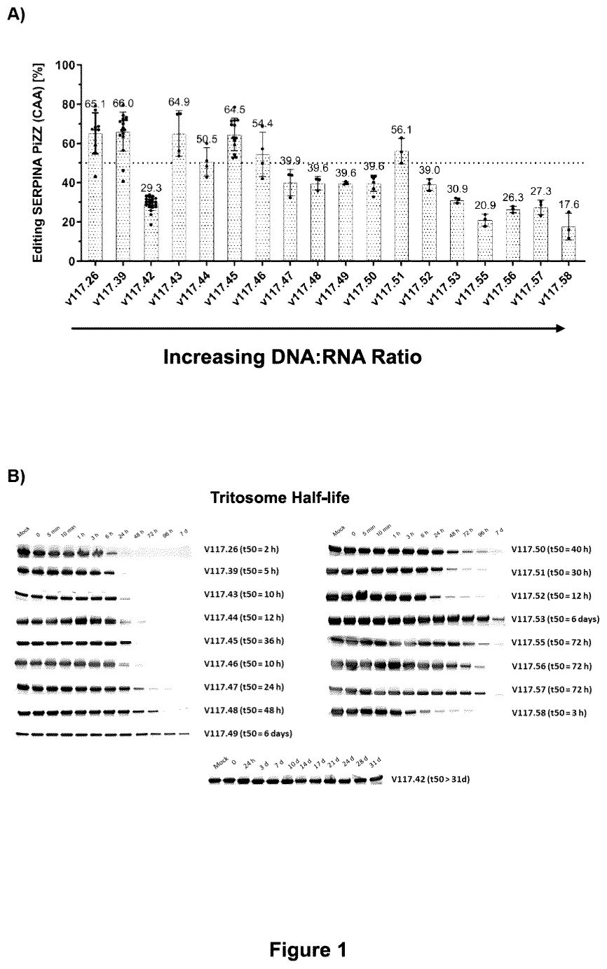

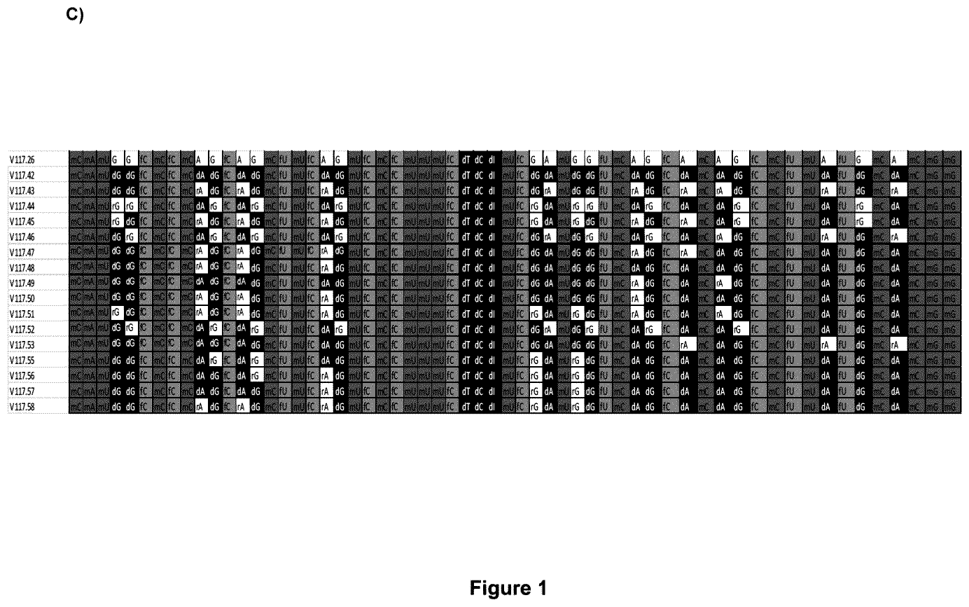

To determine the effect of DNA and RNA on lysosomal stability of the various oligonucleotides, different versions of SERPINA1 E342K targeting constructs were generated (v117.26 to v117.58) and tested in vitro. The various ASOs differ from one another in their DNA and RNA content and are listed in Table 1. The results of Example 1 are shown in FIG. 1. v117.26 served as control.

To assess cellular efficacy, 2.5×10⁴ HeLa cells (Cat. No.: ATCC CCL-2) were seeded in a 24-well plate. After 24 h, cells were forward transfected with a plasmid containing the human SERPINA1 E342K mutated cDNA. 300 ng plasmid and 0.9 μl FuGENE® 6 (Promega) were each diluted in 50 μl Opti-MEM and incubated for 5 min, then combined and incubated for an additional 20 min. The medium was changed, and the transfection mix evenly distributed into one well. 24 h after plasmid transfection, cells were forward transfected with 5 pmol construct/well and 1.5 μl/well Lipofectamine RNAiMAX Reagent (ThermoFisher Scientific). After 24 h, medium was changed. 48 h post transfection, cells were harvested for RNA isolation and sequencing. Lysosomal degradation assay was conducted in mixed gender rat liver tritosomes (tebu-bio, article no. 098R0610.LT). Tritosome solutions were diluted to an acid phosphatase concentration of 0.1115 U/ml with a 20 mM Na-Citrate solution (pH=5.0). In the experiments, 15 pmol of the respective ASO were diluted in the 0.1115 U/ml acid phosphatase tritosome solutions. Mock samples contained 15 pmol ASO diluted in PBS only. All samples were incubated at 37° C. for the given time points, then frozen with liquid nitrogen and stored immediately at −80° C. Degradation of the oligonucleotides was visualized by denaturing urea PAGE. Denaturation of the samples was achieved by adding 7 μl RNA loading dye (1:10 dilution of Rotiphorese® Sequencing gel buffer concentrate in Rotiphorese® Sequencing gel diluent, Carl Roth) to each sample before incubating at 70° C. for 2 min. Denatured samples were subsequently loaded onto a urea (7 M) polyacrylamide (15%) electrophoresis (PAGE) gel and run for 4-6 h at 1200 V in 1×TEE (Tris-borate-EDTA) buffer. Bands were visualized using a SYBR™ Gold Nucleic Acid Gel Stain (ThermoFisher Scientific) according to manufacturer's instructions and scanned at an excitation wavelength λₑₓ=473 nm with a Fujifilm FLA-5100 Fluorescent Image Analyzer. Half-lives were quantified by integration of the greyscale values of each individual full-length ASO band with ImageJ. The construct half-lives were calculated relative to the mock samples

TABLE 1
SERPINA1 E342K targeting construct sequences including nucleobase and
backbone modifications used in Example 1. mN = 2′-O-methyl (2′-OMe), fN = 2′-fluoro (2′-
F), N = 2′-OH (ribose; RNA), dN = 2′-H (deoxyribose; DNA), * = phosphorothioate (PS) linkage;
GalNAc = triantennary N-acetylgalactosamine.

| Construct | | Seq | Fig. |
|---|---|---|---|
| Name | Construct Sequence (5′ to 3′ direction) | ID | No. |
| V117.26 | mC*mA*mU*G*G* fC*mC* fC* mC*A*G* fC* A*G* mC* fU* mU* fC* A*G* | 1 | 1 |
| | mU* fC* mC* fC* mU* fU mU fC dTdCdI mU fC G*A* mU* G*G* fU* mC* | | |
| | A*G* fC* A* mC*A*G* fC*mC* fU*mU* A* fU* G* fC*A* mC*mG*mG | | |
| V117.39 | mC* mA* mU* G* G* fC* mC* fC* mC* A* G* fC* A* G* mC* fU* mU* fC* | 2 | 1 |
| | A* G* mU* fC* mC* fC* mU*mUmUfC* dT*dC*dI mU f C G* A* mU* G* | | |
| | G* fU*mC*A*G*fC*A*mC*A*G*fC*mC* fU* mU* A* fU* G* mC* A* mC* | | |
| | mG* mG | | |
| V117.42 | C6-Aminolinker - | 3 | 1 |
| | mC* mA* mU* dG* dG* fC*mC* fC*mC* dA* dG* fC* dA* dG* mC* fU* | | |
| | mU*fC*dA*dG*mU*fC*mC*fC*mU*mUmU fC* dT*dC*dI mU fC dG*dA* | | |
| | mU*dG*dG*fU*mC*dA*dG*fC*dA*mC*dA*dG*fC*mC*fU*mU*dA*fU*dG* | | |
| | mC*dA*mC*mG*mG | | |
| V117.43 | C6-Aminolinker - mC* mA* mU* dG*dG*fC*mC* fC* mC* A* dG*fC* A* | 4 | 1 |
| | dG*mC*fU*mU*fC*A*dG*mU*fC*mC*fC*mU*mUmUfC* dT*dC*dImU fC | | |
| | dG*A*mU*dG*dG*fU*mC*A*dG*fC*A*mC*A*dG*fC*mC*fU*mU*A*fU*dG* | | |
| | mC*A*mC*mG*mG | | |
| V117.44 | C6-Aminolinker - | 5 | 1 |
| | mC*mA*mU*G*G*fC*mC*fC*mC*dA*G*fC*dA*G*mC*fU*mU*fC*dA*G*m | | |
| | U*fC*mC*fC*mU*mUmUfC* dT*dC*dImU fC G* dA*mU* G* G* fU* mC* | | |
| | dA*G*fC*dA*mC*dA*G*fC*mC*fU*mU*dA*fU*G*mC*dA*mC*mG*mG | | |
| V117.45 | C6-Aminolinker - | 6 | 1 |
| | mC*mA*mU*G*dG*fC*mC*fC*mC*A*dG*fC*A*dG*mC*fU*mU*fC*A*dG* | | |
| | mU*fC*mC*fC*mU*mUmUfC* dT*dC*dImUfC G* dA*mU* G* dG*fU* | | |
| | mC* A* dG*fC* A* mC*dA*G* fC* mC* fU* mU* dA*fU* G* mC* dA*mC* | | |
| | mG* mG | | |
| V117.46 | C6-Aminolinker - | 7 | 1 |
| | mC*mA*mU*dG*G*fC*mC*fC*mC*dA*G*fC*dA*G*mC*fU*mU*fC*dA*G* | | |
| | mU*fC*mC*fC*mU*mUmUfC* dT*dC*dImU fC dG*A* mU* dG*G* fU* | | |
| | mC* dA*G*fC*dA*mC*A*dG*fC*mC*fU*mU*A*fU*dG*mC*A*mC*mG*mG | | |
| V117.47 | mC*mA*mU*dG*dG*fC*mC*fC*mC*A*dG*fC*A*dG*mC*fU*mU*fC*A*dG* | 8 | 1 |
| | mU*fC*mC*fC*mU*mUmUfC* dT*dC*dImU fC dG*dA*mU* dG*dG* fU* | | |
| | mC* A* dG*fC* A*mC* dA*dG*fC* mC* fU* mU* dA*fU* dG*mC* dA*mC* | | |
| | mG* mG | | |
| V117.48 | mC*mA*mU*dG*dG*fC*mC*fC*mC*A*dG*fC*A*dG*mC*fU*mU*fC*A*dG* | 9 | 1 |
| | mU*fC*mC*fC*mU*mUmUfC* dT*dC*dImUfCdG*dA*mU* dG*dG*fU* | | |
| | mC* dA*dG*fC* dA*mC* dA*dG*fC* mC*fU* mU* dA*fU* dG*mC* | | |
| | dA*mC* mG* mG | | |
| V117.49 | mC*mA*mU*dG*dG*fC*mC*fC*mC*dA*dG*fC*dA*dG*mC*fU*mU*fC*dA | 10 | 1 |
| | *dG*mU*fC*mC*fC*mU*mUmUfC* dT*dC*dImU fC dG*dA*mU* | | |
| | dG*dG*fU* mC* A* dG*fC*dA*mC*A*dG*fC* mC*fU* mU* dA*fU* | | |
| | dG*mC* dA*mC* mG* mG | | |
| V117.50 | mC*mA*mU*dG*dG*fC*mC*fC*mC*A*dG*fC*A*dG*mC*fU*mU*fC*A*dG* | 11 | 1 |
| | mU*fC*mC*fC*mU*mUmUfC* dT*dC*dImU fC dG*dA*mU* dG*dG*fU* | | |
| | mC* A* dG*fC* dA*mC*dA*dG*fC* mC*fU*mU*dA*fU* dG*mC* dA*mC* | | |
| | mG* mG | | |
| V117.51 | mC*mA*mU*G*dG*fC*mC*fC*mC*A*dG*fC*A*dG*mC*fU*mU*fC*A*dG* | 12 | 1 |
| | mU*fC*mC*fC*mU*mUmUfC* dT*dC*dI mUfC G*dA*mU*G*dG*fU* mC* | | |
| | A*dG*fC*dA*mC*A*dG*fC*mC*fU*mU*dA*fU*dG*mC*dA*mC*mG*mG | | |
| V117.52 | mC*mA*mU*dG*G*fC*mC*fC*mC*dA*G*fC*dA*G*mC*fU*mU*fC*dA*G* | 13 | 1 |
| | mU*fC*mC*fC*mU*mUmUfC* dT*dC*dImUfC dG*A* mU* dG*G* fU*mC* | | |
| | dA*G*fC*dA*mC*dA*G*fC*mC*fU*mU*dA*fU*dG*mC*dA*mC*mG*mG | | |
| V117.53 | mC*mA*mU*dG*dG*fC*mC*fC*mC*dA*dG*fC*dA*dG*mC*fU*mU*fC*dA | 14 | 1 |
| | *dG*mU*fC*mC*fC*mU*mUmUfC* dT*dC*dImUfCdG*dA*mU* | | |
| | dG*dG*fU* mC*dA*dG*fC*A* mC* dA*dG*fC*mC* fU* mU* A* fU* dG* | | |
| | mC* A* mC*mG*mG | | |
| V117.54 | mC*mA*mU*dG*dG*fC*mC*fC*mC*dA*dG*fC*dA*dG*mC*fU*mU*fC*dA | 15 | 1 |
| | *dG*mU*fC*mC*fC*mU*mUmUfC* dT*dC*dImU fC dG*dA*mU* dG*dG* | | |
| | fU* mC*dA*dG*fC* dA*mC* dA*dG*fC*mC* fU* mU* | | |
| | dA*fU*dG*mC*dA*mC*mG*mG - GalNAc | | |
| V117.55 | mC*mA*mU*dG*dG*fC*mC*fC*mC*dA*G*fC*dA*G*mC*fU*mU*fC*dA* | 16 | 1 |
| | dG*mU*fC*mC*fC*mU*mUmUfC* dT*dC*dImUfCG*dA*mU*G*dG*fU* | | |
| | mC* dA*dG*fC* dA*mC* dA*dG*fC* mC* fU* mU* dA*fU* dG*mC* dA* | | |
| | mC* mG* mG | | |
| V117.56 | mC*mA*mU*dG*dG*fC*mC*fC*mC*dA*dG*fC*dA*G*mC*fU*mU*fC*A* | 17 | 1 |
| | dG*mU*fC*mC*fC*mU*mUmUfC* dT*dC*dImUfCG*dA*mU*G*dG*fU* | | |
| | mC* dA*dG*fC* dA*mC* dA*dG*fC* mC*fU* mU* dA*fU* dG*mC* | | |
| | dA*mC*mG*mG | | |
| V117.57 | mC*mA*mU*dG*dG*fC*mC*fC*mC*dA*dG*fC*dA*dG*mC*fU*mU*fC*A* | 18 | 1 |
| | dG*mU*fC*mC*fC*mU*mUmUfC* dT*dC*dImUfCG*dA*mU*G*dG*fU* | | |
| | mC* dA*dG*fC*dA*mC*dA*dG*fC* mC* fU* mU* dA*fU* dG*mC* | | |
| | dA*mC*mG*mG | | |
| V117.58 | mC*mA*mU*dG*dG*fC*mC*fC*mC*A*dG*fC*A*dG*mC*fU*mU*fC*A*dG* | 19 | 1 |
| | mU*fC*mC*fC*mU*mUmUfC* dT*dC*dImUfCG*dA*mU*G*dG*fU* mC* | | |
| | dA*dG*fC*dA*mC*dA*dG*fC*mC*fU*mU*dA*fU*dG*mC*dA*mC*mG* | | |
| | mG | | |

As shown in FIG. 1A, incorporation of increasing amounts of DNA into the oligonucleotide backbone, thus increasing the DNA-to-RNA (DNA:RNA) ratio, led to an overall decrease in SERPINA1 E342K editing yields. Two control constructs were used: v117.26 and v117.39 (5.1% DNA overall). These ASOs differ from one another in that v117.39 differs from v117.26 in that it contains additional phosphorothioate linkages at position d, e, and f (Table 1). The inclusion of the additional PS linkages at the CBT alone was able to improve the lysosomal half-life (t₅₀) of the embodiments from 2 h (v117.26) to 5 h (v117.39). However, an even longer lysosomal half-life would be more preferable from a therapeutic perspective. ASO v117.42 (39% DNA), wherein all remaining RNA nucleosides were replaced with DNA, showed low editing yields compared to the v117.26 control construct that carried no DNA outside the CBT. Interestingly, constructs that contained a higher DNA:RNA ratio in their backbone (e.g., v117.53 (33.9% DNA) to v117.58 (30.5%)) showed lower levels of RNA editing when compared to constructs with a smaller DNA:RNA ratio (e.g., v117.43 (23.7% DNA) to v117.46 (22% DNA)).

Furthermore, incorporating increasing amounts of DNA into the ASO backbone, thus increasing the DNA:RNA ratio of the ASO, led to higher lysosomal stability (FIG. 1B). This stability peaks when all remaining RNA nucleosides are replaced by DNA: ASO v117.42 (control DNA backbone; 39% DNA) showed the highest degree of stability, achieving a half-life exceeding 31 days (t₅₀>d31). Constructs v117.49 and v117.53 were able to withstand 10% fetal bovine serum (FBS) and also showed high levels of lysosomal stability up until about day 7 (7 d) with a half-life (t₅₀) of 6 days. On the other hand, v117.39, which lacks chemical modifications at many 2′-hydroxyl group of the nucleoside sugar moiety was rapidly degraded by 6 h (t₅₀=5 h). While the stability (t₅₀) of both ASOs against degradation was improved compared to v117.39, the editing efficacy was reduced to 39.6% and 30.9% respectively (see, FIGS. 1A and B).

Overall, these data show that increasing the amount of DNA (DNA:RNA ratio) outside of the CBT in oligonucleotides carrying 2′-OMe- and 2′-F-modifications at the +2 and +3 positions, respectively, wherein h and i are not PS linkages, and wherein no more than 6 consecutive nucleotides have the same 2′-modification tends to decrease editing efficacy. At the same time, increasing the DNA:RNA ratio improves lysosomal stability and thus durability against serum RNase degradation, thus promoting longevity of the different constructs in serum. However, placement of a very high degree of DNA interferes with editing efficiency. Nevertheless, potentially useful embodiments that contain a notable number of 2′-unmodified ribonucleotides can be created that have a reasonable balance of stability versus editing efficiency and also have a relatively low content of (potentially toxic) 2′-F (e.g., 20% in v117.53) and 2′-O-methyl (e.g., about 35% in v117.49), even though 2′-F and 2′-O-methyl are typically required to some degree to stabilize the ASO (e.g., at least 10% of all nucleosides are 2′-F and another at least 10% are 2′-O-methyl-modified). However, for many applications further stabilization is desired. Hence, without being bound by any particular theory, the inventors submit that there must be an optimal balance between a mixture of 2′-F, 2′-OMe-nucleoside modifications, PS linkage modifications, and the target editing efficacy and lysosomal stability of the oligonucleotide, which may be achieved by replacement of the RNA nucleosides with DNA nucleosides.

### Example 2. Editing Efficacy of SERPINA1 E342K Targeting Oligonucleotides by Mixing 2′-F- and 2′-OMe-Modifications and DNA

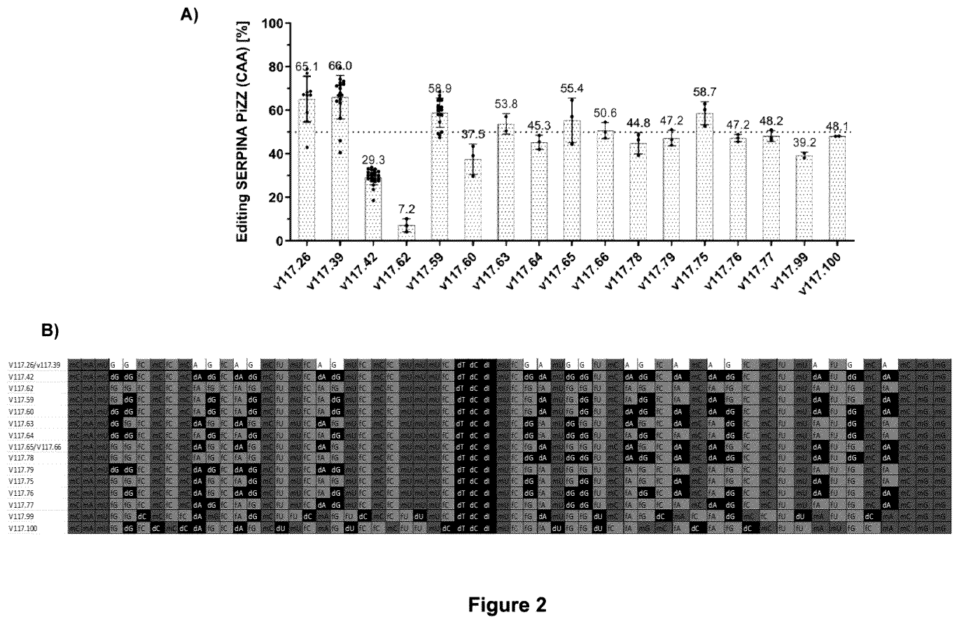

To investigate the stabilization effect of 2′-F- and 2′-OMe backbone modifications relative to and in combination with DNA content more thoroughly, different oligonucleotides were generated and assayed for their in vitro RNA editing efficacy according to the protocols described in Example 1. The different ASO constructs tested are listed in Table 2. Results are shown in FIG. 2. As described for Example 1, v117.26 and v117.39 were used as controls.

As shown in FIG. 2A, high levels of 2′-F modification is associated with a decrease in editing efficacy. Oligonucleotides containing high levels of DNA content (v117.42; 29.3%, containing 39% DNA) or 2′-F-modified nucleosides (v117.62; 7.2%, containing 58% 2′-F) showed a drastic decrease in editing efficacy. On the other hand, constructs that carried a more balanced amount of 2′-F-modifications and DNA showed an RNA editing efficacy of between about 37.5% (v117.60) and 58.9% (v117.59), which was comparable to that of the control. These data confirm that a balanced mixture of 2′-F-, 2′-OMe-, and 2′-H-modifications may be used to compensate for the replacement of 2′-OH modifications and that containing excess amounts of a single type of modification, i.e., 2′-F (v117.62) or 2′-H (v117.42), has a detrimental effect on overall RNA editing. A certain preference for more 2′-F than DNA could also be observed. Embodiments that contained higher amounts of 2′-F usually performed better than embodiments with more DNA, e.g., v117.65 (55.4% editing, containing 42% 2′-F and 20% DNA) versus v117.60 (37.5% editing, containing 32% 2′-F and 31% DNA). Also, although containing 39% DNA and only 24% 2′-F, v117.42 still achieved an editing efficacy of 29.3%, which may be sufficient when combined with its desirably high lysosomal stability (see Example 1). The 2′-OMe amount of all embodiments remained between 32-37% (e.g., v117.99 and v117.59, respectively).

To further determine whether the 2′-sugar modifications outside of the CBT and the end-blocked termini were position-dependent or -independent of the nature of the nucleotide (purine versus pyrimidine), ASOs v117.99 and v117.100, both of which comprise the same amount of overall 2′-F-, 2′-OMe-, and 2′-H-modifications (25.4% DNA, 42.4% 2′-F and 32.2% 2′-OMe), were assessed for their RNA editing efficacy. The positions of all 2′-sugar modifications (2′-F, 2′-OMe, and DNA) were randomised apart from the three modifications at each of the two termini (the “endblocks”), the CBT, and the N₊₂ and N₊₃ positions of the extended hotspot (see, e.g., Example 14 for extended hotspot). The linkage backbone modifications were not changed compared to all other embodiments. The results were subsequently compared to the parent reference sequence v117.59. As shown in FIG. 2A, v117.99 and 177.100 showed good editing (39.2% and 48.1%, respectively), indicating that the RNA editing efficacy was not significantly affected by the relative positioning of the 2′-modifications.

TABLE 2
SERPINA1 E342K targeting construct sequences and modifications used in
Example 2. mN = 2′-O-methyl (2′-OMe), fN = 2′-fluoro (2′-F), N = 2′-OH (ribose; RNA), dN =
2′-H (deoxyribose; DNA), * = phosphorothioate (PS) linkage, GalNAc = triantennary N-
acetylgalactosamine

| Construct | | Seq. | Fig. |
|---|---|---|---|
| Name | Construct Sequence (5′ to 3′ direction) | ID | No. |
| V117.26 | mC*mA*mU*G*G* fC*mC*fC*mC*A*G* fC* A* G* mC* fU* mU* fC* | 1 | 2 |
| | A*G*mU*fC*mC*fC*mU*fUmUfC dTdCdI mU fC G* A* mU* G* G* | | |
| | fU*mC*A*G*fC*A*mC*A*G*fC*mC*fU*mU*A*fU*G*mC*A*mC*mG*mG | | |
| V117.39 | mC*mA*mU*G*G* fC*mC*fC*mC*A*G* fC* A* G* mC* fU* mU* fC* | \|2 | 2 |
| | A*G*mU*fC*mC*fC*mU*mUmUfC* dT*dC*dI mU fC G* A* mU* G* G* | | |
| | fU*mC*A*G*fC*A*mC*A*G*fC*mC*fU*mU*A*fU*G*mC*A*mC*mG*mG | | |
| V117.42 | C6-Aminolinker - | 3 | 2 |
| | mC*mA*mU*dG*dG*fC*mC*fC*mC*dA*dG*fC*dA*dG*mC*fU*mU*fC* | | |
| | dA*dG*mU*fC*mC*fC*mU*mUmUfC* dT*dC*dImU fC dG*dA*mU* | | |
| | dG*dG*fU*mC*dA*dG*fC*dA*mC*dA*dG*fC*mC*fU*mU*dA*fU*dG* | | |
| | mC*dA*mC*mG*mG | | |
| V117.59 | mC*mA*mU*fG*dG*fC*mC*fC*mC*fA*dG*fC*fA*dG*mC*fU*mU*fC*fA | 20 | 2 |
| | *dG*mU*fC*mC*fC*mU*mUmUfC* dT*dC*dImU fC fG*dA*mU* | | |
| | fG*dG*fU*mC*fA*dG*fC*fA*mC*dA*fG*fC*mC*fU*mU*dA*fU*fG*mC* | | |
| | dA*mC*mG*mG | | |
| V117.60 | mC*mA*mU*dG*dG*fC*mC*fC*mC*fA*dG*fC*fA*dG*mC*fU*mU*fC* | 21 | 2 |
| | fA*dG*mU*fC*mC*fC*mU*mUmUfC* dT*dC*dImU fC fG* dA*mU* fG* | | |
| | dG*fU*mC*dA*dG*fC*dA*mC*dA*dG*fC*mC*fU*mU*dA*fU*dG*mC* | | |
| | dA*mC*mG*mG | | |
| V117.62 | mC*mA*mU*fG*fG*fC*mC*fC*mC*fA*fG*fC*fA*fG*mC*fU*mU*fC*fA* | 22 | 2 |
| | fG*mU*fC*mC*fC*mU*mUmUfC*dT*dC*dImUfCfG*fA*mU*fG*fG*fU* | | |
| | mC*fA*fG*fC*fA*mC*fA*fG*fC*mC*fU*mU*fA*fU*fG*mC*fA*mC*mG*mG | | |
| V117.63 | mC*mA*mU*dG*fG*fC*mC*fC*mC*dA*fG*fC*dA*fG*mC*fU*mU*fC* | 23 | 2 |
| | dA*fG*mU*fC*mC*fC*mU*mUmUfC*dT*dC*dImUfCdG*fA*mU*dG*fG* | | |
| | fU*mC*dA*fG*fC*dA*mC*fA*dG*fC*mC*fU*mU*fA*fU*dG*mC*fA*mC* | | |
| | mG*mG | | |
| V117.64 | mC*mA*mU*dG*dG*fC*mC*fC*mC*fA*dG*fC*fA*dG*mC*fU*mU*fC* | 24 | 2 |
| | fA*dG*mU*fC*mC*fC*mU*mUmUfC*dT*dC*dImUfCdG*fA*mU*dG* | | |
| | dG* fU* mC* fA*dG*fC*fA*mC* fA*dG*fC*mC* fU*mU*fA* fU* dG*mC* | | |
| | fA* mC* mG*mG | | |
| V117.65 | mC*mA*mU*fG*fG*fC*mC*fC*mC*dA*fG*fC*dA*fG*mC*fU*mU*fC*dA | 25 | 2 |
| | *fG*mU*fC*mC*fC*mU*mUmUfC*dT*dC*dImUfCfG*dA*mU*fG*fG*fU | | |
| | *mC*dA*fG*fC*dA*mC*dA*fG*fC*mC*fU*mU*dA*fU*fG*mC*dA*mC* | | |
| | mG*mG | | |
| V117.66 | mC*mA*mU*fG*dG*fC*mC*fC*mC*fA*dG*fC*fA*dG*mC*fU*mU*fC*fA | 26 | 2 |
| | *dG*mU*fC*mC*fC*mU*mUmUfC* dT*dC*dImU fC fG* dA*mU* fG* | | |
| | dG*fU*mC*fA*dG*fC*fA*mC*dA*fG*fC*mC*fU*mU*dA*fU*fG*mC*dA* | | |
| | mC*mG*mG - GalNAc | | |
| V117.75 | mC*mA*mU*fG*fG*fC*mC*fC*mC*dA*fG*fC*fA*dG*mC*fU*mU*fC*fA* | 27 | 2 |
| | fG*mU*fC*mC*fC*mU*mUmUfC* dT*dC*dI mU fC dG*fA* mU* fG* | | |
| | dG*fU*mC*fA*fG*fC*dA*mC*fA*fG*fC*mC*fU*mU*dA*fU*fG*mC*fA* | | |
| | mC*mG*mG | | |
| V117.76 | mC*mA*mU*fG*dG*fC*mC*fC*mC*dA*fG*fC*dA*dG*mC*fU*mU*fC* | 28 | 2 |
| | fA*dG*mU*fC*mC*fC*mU*mUmUfC* dT*dC*dI mU fC dG*fA*mU* | | |
| | dG*dG*fU*mC*fA*dG*fC*dA*mC*fA*dG*fC*mC*fU*mU*dA*fU*fG*mC | | |
| | *dA*mC*mG*mG | | |
| V117.77 | mC*mA*mU*fG*fG*fC*mC*fC*mC*dA*dG*fC*fA*fG*mC*fU*mU*fC*dA | 29 | 2 |
| | *dG*mU*fC*mC*fC*mU*mUmUfC* dT*dC*dI mU fC fG* fA* mU* | | |
| | dG*dG*fU*mC*fA*fG*fC*fA*mC*dA*dG*fC*mC*fU*mU*fA*fU*fG*mC* | | |
| | dA*mC*mG*mG | | |
| V117.78 | mC*mA*mU*fG*fG*fC*mC*fC*mC*fA*fG*fC*A*fG*mC*fU*mU*fC*fA* | 30 | 2 |
| | fG*mU*fC*mC*fC*mU*mUmUfC* dT*dC*dImU fC dG*dA* mU* | | |
| | dG*dG*fU* mC* dA*dG*fC* dA*mC* dA*dG*fC* mC* fU* mU* dA*fU* | | |
| | dG*mC*dA*mC*mG*mG | | |
| V117.79 | mC*mA*mU*dG*dG*fC*mC*fC*mC*dA*dG*fC*dA*dG*mC*fU*mU*fC* | 31 | 2 |
| | dA*dG*mU*fC*mC*fC*mU*mUmUfC* dT*dC*dImU fC fG* fA* mU* | | |
| | fG* fG* fU* mC* fA* fG* fC*fA* mC*fA* fG*fC* mC* fU* mU* | | |
| | fA*fU*fG*mC*fA*mC*mG*mG | | |
| V117.99 | mC*mA*mU*fG*fG*dC*mC*fC*fC*dA*mG*fC*fA*dG*mC*fU*fU*dC* | 32 | 2 |
| | mA*fG*fU*dC*mC*fC*fU*dUmUfC* dT*dC*dImUfCfG*dA*mU* fG* fG* | | |
| | dU*mC*fA*fG*dC*mA*fC*fA*dG*mC*fC*fU*dU*mA*fU*fG*dC*mA*mC | | |
| | *mG*mG | | |
| V117.100 | mC*mA*mU*fG*dG*fC*dC*mC*dC*dA*fG*fC*dA*fG*mC*dU*mU*fC* | 33 | 2 |
| | mA*fG*dU*fC*fC*mC*fU*fUmUdC* dT*dC*dImUfC fG* fA* dU*fG* fG* | | |
| | dU*fC*fA*mG*mC*fA*dC*fA*fG*dC*mC*fU*fU*mA*mU*fG*fC*mA*mC | | |
| | *mG*mG | | |

In summary, the data suggest that high levels of only one type of 2′-modification (e.g., high levels of 2′-F- or 2′-H modifications) has a detrimental effect on the overall editing efficacy of the oligonucleotide (cf. v117.42 and v117.62). Generally, the highest performing long (>40 nt, here: 59 nt) embodiments (that achieved >45% editing) contained more than 35% 2′-F modifications and did not exceed a DNA content of 25%, suggesting these may be preferred 2′ modification amounts. However, a certain tolerance for more DNA (up to approx. 40%) and fewer 2′-F modifications (down to approx. 25%) can be observed and may be dependent on other factors such as the embodiment's nucleobase sequence. Also, the data suggest that the overall amount of certain 2′-modifications has a stronger influence on the corresponding editing yields than the precise position of each 2′-modification within the oligonucleotide.

### Example 3. Editing Efficacy and Lysosomal Stabilization of STAT1 Y701 Targeting Oligonucleotides

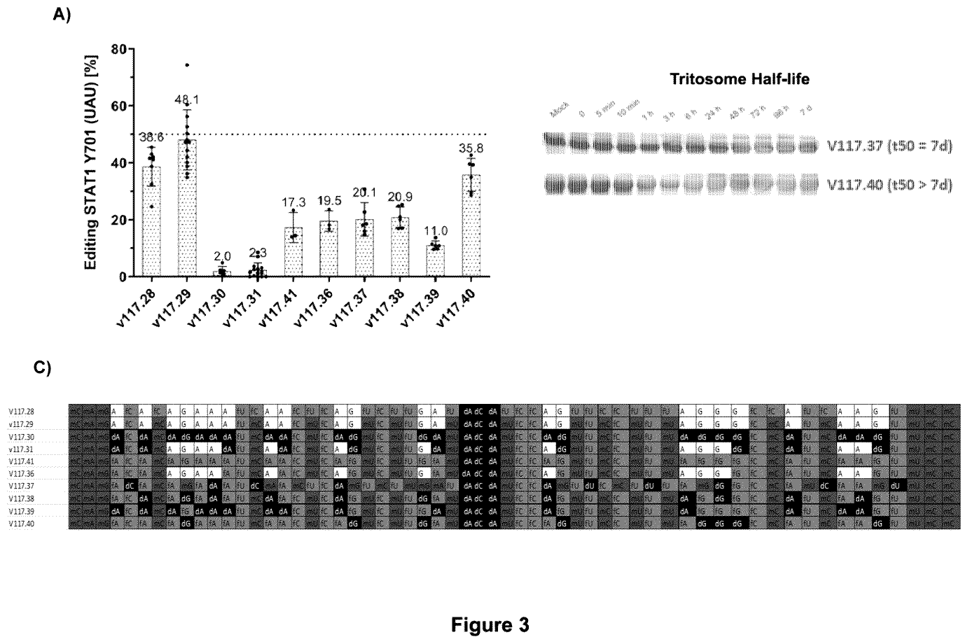

Analogous to the evaluation of SERPINA1 E342K targeting ASOs for their RNA editing efficacy and lysosomal stability, editing efficacy and lysosomal stability was determined for a variety of human STAT1 Y701 targeting ASOs. The different ASO construct sequences and respective modifications are listed in Table 3. The results are shown in FIG. 3. Constructs v117.28 and v117.29 (having 2′-F- and 2′-OMe-modifications but no 2′-H outside of the CBT) were used as control.

To assess cellular efficacy, 1×10⁵ HeLa cells were forward transfected with 25 pmol construct and 1.5 μl Lipofectamine RNAiMAX Reagent (ThermoFisher Scientific) per 24-well according to the Lipofectamine RNAiMAX reagent protocol. RNA was isolated 24 h later for Sanger sequencing. Lysosomal degradation assays were determined as described in Example 1.

The data show a trend similar to the one seen with the SERPINA1 E342K specific oligonucleotides. Replacing 2′-OH groups (RNA) with 2′-H groups (DNA), thus increasing the DNA:RNA ratio, resulted in a general decrease in editing efficacy down to 2% for v117.30 and 2.3% for v117.31 (each containing 44% and 22% DNA, respectively) (FIG. 3A). Similarly, comprising high levels of 2′-F-modifications also led to low levels of RNA editing (17.36 and v17.41, containing 41% and 63% 2′-F modifications, respectively). Similarly, v117.39, which contains a relatively higher 2′-H content when compared to, e.g., v117.83 showed editing yields of only 11%. On the other hand, ASOs comprising a combination of 2′-F, 2′-OMe and 2′-H-modifications showed enhanced editing capacity. However, these embodiments only performed similarly to the controls when containing approx. double the amount of 2′-F modifications compared to DNA, which is seen for construct v117.40 (comprising 49.2% 2′-F, 18.6% DNA and 32.2% 2′-OMe). V117.40 showed enhanced editing efficacy (35.8%) and lysosomal stability (t₅₀>7 d vs t₅₀=7 d) when compared to v117.37 (FIGS. 3A and B).

TABLE 3
STAT1 Y701 targeting construct sequences and modifications used in
Example 3. mN = 2′-O-methyl (2′-OMe), fN = 2′-fluoro (2′-F), N = 2′-OH (ribose; RNA), dN =
2′-H (deoxyribose; DNA), * = phosphorothioate (PS) linkage, GalNAc = triantennary N-
acetylgalactosamine.

| Construct | | Seq. | Fig. |
|---|---|---|---|
| Name | Construct Sequence (5′ to 3′ direction) | ID | No. |
| V117.28 | mC*mA*mG*A*fC*A*fC*A*G*A*A*A*fU*fC*A*A*fC*fU*fC*A*G*fU*fC* | 34 | 3 |
| | fU*fU*GAfU dAdCdA fU fC fC* A* G* fU* fU* fC* fC* fU* fU* fU*A* G* | | |
| | G* G* fC* fC* A* fU* fC* A*A*G*fU*mU*mC*mC | | |
| V117.29 | mC*mA*mG*A*fC*A*mC*A*G*A*A*A*fU*mC*A*A*fC*mU*fC*A*G*mU* | 35 | 3 |
| | fC*mU*fU*GAmU* dA*dC*dA mUfC fC* A* G* mU* fU* mC* fC* | | |
| | mU*fU* mU* A* G* G* G* fC* mC* A* fU* mC*A* A* G* fU* mU* mC* | | |
| | mC - GalNAc | | |
| V117.30 | C6-Aminolinker - | 36 | 3 |
| | mC*mA*mG*dA*fC*dA*mC*dA*dG*dA*dA*dA*fU*mC*dA*dA*fC*mU | | |
| | *fC*dA*dG*mU*fC*mU*fU*dGdAmU* dA*dC*dA mUfC fC* | | |
| | dA*dG*mU*fU*mC*fC*mU*fU*mU*dA*dG*dG*dG*fC*mC*dA*fU*mC* | | |
| | dA*dA*dG*fU*mU*mC*mC | | |
| V117.31 | C6-Aminolinker - | 37 | 3 |
| | mC*mA*mG*dA*fC*dA*mC*A*G*A*A*dA*fU*mC*A*dA*fC*mU*fC*A* | | |
| | dG*mU*fC*mU*fU*GdAmU* dA*dC*dA mUfC fC*A* dG*mU* fU* | | |
| | mC*fC*mU*fU*mU*A*G*G*dG*fC*mC*dA*fU*mC*A*A*dG*fU*mU*mC | | |
| | *mC | | |
| V117.36 | C6-Aminolinker - | 38 | 3 |
| | mC*mA*mG*fA*fC*fA*mC*A*G*A*A*A*fU*mC*A*fA*fC*mU*fC*A*fG* | | |
| | mU*fC*mU*fU*GfAmU* dA*dC*dA mUfC fC*A*fG* mU* fU* mC* | | |
| | fC*mU*fU*mU*A*G*G*fG*fC*mC*fA*fU*mC*A*A*fG*fU*mU*mC*mC | | |
| V117.37 | mC*mA*mG*fA*dC*fA*mC*fA*mG*fA*dA*fA*fU*dC*mA*fA*mC*fU*fC* | 39 | 3 |
| | dA*mG*fU*mC*fU*mU*mGmAfU* dA*dC*dA mU fC fC* dA* | | |
| | mG*fU*dU*fC*mC*fU*dU*fU*fA*mG*dG*fG*fC*mC*fA*mU*dC*fA*fA* | | |
| | mG*dU*mU*mC*mC | | |
| V117.38 | mC*mA*mG*fA*fC*dA*mC*fA*dG*fA*dA*fA*fU*mC*dA*fA*fC*mU*fC* | 40 | 3 |
| | dA*fG*mU*fC*mU*fU*dGfAmU* dA*dC*dA mUfC fC*dA*fG* mU* | | |
| | fU* mC* fC* mU*fU*mU*dA*fG*dG*fG*fC*mC* dA*fU*mC*fA* dA*fG* | | |
| | fU*mU*mC*mC | | |
| V117.39 | mC*mA*mG*dA*fC*dA*mC*dA*fG*dA*dA*dA*fU*mC*dA*dA*fC*mU* | 41 | 3 |
| | fC*dA*fG*mU*fC*mU*fU*fGdAmU* dA*dC*dA mUfC fC* | | |
| | dA*fG*mU*fU*mC*fC*mU*fU*mU*dA*fG*fG*fG*fC*mC*dA*fU*mC*dA | | |
| | *dA*fG*fU*mU*mC*mC | | |
| V117.40 | mC*mA*mG*fA*fC*fA*mC*fA*dG*fA*fA*fA*fU*mC*fA*fA*fC*mU*fC*fA* | 42 | 3 |
| | dG*mU*fC*mU*fU*dGfAmU* dA*dC*dA mUfC fC* fA* dG*mU* | | |
| | fU*mC*fC*mU*fU*mU*fA*dG*dG*dG*fC*mC*fA*fU*mC*fA*fA*dG*fU* | | |
| | mU*mC*mC | | |
| V117.41 | mC*mA*mG*fA*fC*fA*mC*fA*fG*fA*fA*fA*fU*mC*fA*fA*fC*mU*fC*fA* | 43 | 3 |
| | fG*mU*fC*mU*fU*fGfAmU*dA*dC*dAmUfCfC*fA*fG*mU*fU*mC*fC* | | |
| | mU*fU*mU*fA*fG*fG*fG*fC*mC*fA*fU*mC*fA*fA*fG*fU*mU*mC*mC | | |

As before, the data indicate that optimal editing efficacies can be obtained when combining 2′-F and 2′-OMe-modifications together with 2′-H groups within the ASO sugar moiety, and whereby a 2′-O-alkyl-modification is at position +2 (N₊₂) and a 2′-F-modification is at position +3 (N₊₃), and the ASOs contains no more than 6 consecutive nucleotides with the same 2′ modifications. While a combination of the particular 2′-modifications provided the highest editing, there was a trend to preferentially including more 2′-F-modified nucleosides than DNA (v117.40 with 35.8% editing efficacy compared to v117.39 with 11% editing efficacy). There is also a greater tolerance for a high 2′-F content (up to approx. 50% for v117.40) and less DNA (approx. 19% for v117.40) in the STAT1 Y701 targeting constructs compared to the SERPINA1 E342K targeting constructs.

Overall, the data confirm the results observed with SERPINA1 E342K targeting ASOs, in that certain levels of 2′-F and 2′-OMe modifications should be included and that blocks of continuous uniform sugar modifications should be avoided. In all embodiments that no longer contained natural RNA, there was no block of more than six continuous 2′-F, 2′O-Me or DNA nucleosides. More importantly, these data suggest that specific modification patterns can be transferred to different targets (e.g., STAT1 Y701 v117.37 pattern is taken from SERPINA1 E342K v117.59). That is, while general modification patterns are transferable to a good degree, they may still require some additional target-specific adaptations in order to provide ASOs with optimal editing capacity.

### Example 4. Editing Efficacy and Lysosomal Stabilization of CRB1 C948Y Targeting Oligonucleotides

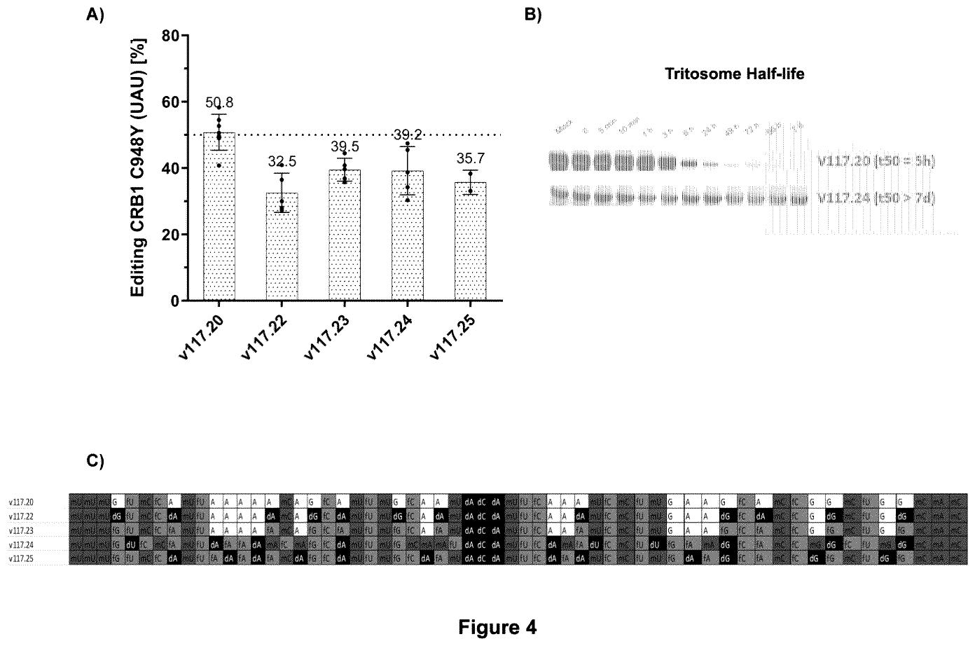

To further determine the combined effect of 2′-F-, 2′-OMe- and 2′-H-modifications on the editing efficacy and stabilization of other genetic targets, different human CRB1 C948Y targeting oligonucleotides were generated and tested. The different construct sequences and their modifications are listed in Table 4. The results for Example 4 are shown in FIG. 4. Construct v117.20, having no 2′-H (DNA) outside the CBT, served as control.

Cellular efficacy was assessed by forward transfecting 300 ng plasmid (containing the CRB1 C948Y cDNA) and 0.9 μl FuGENE® 6 onto 5×10⁴ HeLa cells seeded 24 h prior to transfection in a 24-well. Medium was changed every 24 h. 24 h after plasmid transfection, 25 pmol of construct were forward transfected with 1.5 μl Lipofectamine RNAiMAX (ThermoFisher Scientific) according to the Lipofectamine RNAiMAX protocol. After 24 h incubation, cells were harvested for RNA isolation and Sanger sequencing. Lysosomal degradation assays were performed as described in Example 1.

TABLE 4
CRB1 C948Y targeting construct sequences and modifications used in
Example 4. mN = 2′-O-methyl (2′-OMe), fN = 2′-fluoro (2′-F), N = 2′-OH (ribose; RNA), dN =
2′-H (deoxyribose; DNA), * = phosphorothioate (PS) linkage.

| Construct | | Seq. | Fig. |
|---|---|---|---|
| Name | Construct Sequence (5′ to 3′ direction) | ID | No. |
| V117.20 | mU*mU*mU*G*fU*mC*fC*A*mU*fU*A*A*A*A*A*mC*A*G*fC*A*mU*fU | 44 | 4 |
| | *mU*G*fC* AAmU* dA*dC*dA mUfUfC* A*A* A* mU* fC* mC*fU* mU* | | |
| | G* A* A* G* fC* A* mC* fC* G* G* mC* fU* G* G* mC* mA* mC | | |
| V117.22 | mU*mU*mU*dG*fU*mC*fC*dA*mU*fU*A*A*A*A*dA*mC*A*dG*fC*dA | 45 | 4 |
| | *mU*fU*mU*dG*fC* AdAmU* dA*dC*dA mUfUfC*A* A* dA*mU* fC* | | |
| | mC* fU* mU* G* A* A* dG*fC* dA*mC* fC* G* dG*mC* fU* G* | | |
| | dG*mC* mA*mC | | |
| V117.23 | mU*mU*mU*fG*fU*mC*fC*fA*mU*fU*A*A*A*A*fA*mC*A*fG*fC*fA*mU | 46 | 4 |
| | *fU*mU*fG*fC* AfAmU* dA*dC*dA mUfUfC*A * A* fA* mU* fC* mC* | | |
| | fU* mU* G* A* A* fG* fC* fA* mC* fC* G* fG* mC* fU* G* fG* mC* | | |
| | mA*mC | | |
| V117.24 | mU*mU*mU*fG*dU*fC*mC*fA*mU*fU*dA*fA*fA*dA*mA*fC*mA*fG*fC | 47 | 4 |
| | *dA*mU*fU*mU*fG*mC* mAmAfU* dA*dC*dA mUfUfC*dA* mA* fA* | | |
| | dU*fC* mC* fU* dU*fG* fA* mA* dG*fC* fA* mC* fC* mG* | | |
| | dG*fC*fU*mG*dG*mC*mA*mC | | |
| V117.25 | mU*mU*mU*fG*fU*mC*fC*dA*mU*fU*fA*dA*fA*dA*fA*mC*dA*fG*fC | 48 | 4 |
| | *dA*mU*fU*mU*fG*fC* dAfAmU* dA*dC*dA mUfUfC* dA* fA* | | |
| | dA*mU* fC* mC* fU* mU* fG* dA*fA* dG*fC* fA* mC* fC* | | |
| | dG*fG*mC*fU*dG*fG*mC*mA*mC | | |

As shown in FIG. 4A, while replacing some of the 2′OH groups in v117.20 with 2′H groups only led to a slight decrease in ASO editing efficacy from 50.8% (v117.20, containing 5.1% DNA and 22% 2′-F) to 32.5% (v117.22, containing 25.4% DNA), good editing was still maintained. Similar observations were made for the replacement of 2′OH groups in v117.20 with 2′-F-modifications (V117.23; 39.5%, 2′-F levels increased to 42.4%). Notably, introducing a mixture of 2′-F-, 2′-OMe-, and 2′-H modifications led to similar editing efficacies (v117.24 and v117.25; containing 40.7% 2′-F, 22% DNA, 37.3% 2′-OMe and 44.1% 2′-F, 25.4% DNA, 30.5% 2′-OMe, respectively). However, with respect to lysosomal stabilisation, it was observed that introducing a mixture of 2′-F-, 2′-OMe-, and 2′-H modifications led to an overall improvement. While construct v117.20 had a t₅₀ of 5 h, v117.24 achieved a t₅₀>7 d, indicating that a mixture of 2′-F/2′-OMe/DNA replacing all native RNA nucleosides promotes ASO stabilisation (FIG. 4A).

These data again demonstrate that well balanced mixtures of 2′-F and 2′-OMe modifications (about 40-45% 2′-F, 22-25% DNA and 31% 2′-OMe) avoiding blocks of continuous uniform sugar modifications gave efficient editing yields and lysosomal stabilisation. Also, as already observed in Example 3, the length of uniform 2′-modification blocks does not exceed 6 nt, or even 3 nt. Furthermore, these data again suggest that this combination of 2′-modifications can be applied to different oligonucleotide sequences and thus used independently of the actual target (e.g., CRB1 C948Y v117.24 pattern is taken from SERPINA1 E342K v117.59).

### Example 5. Editing Efficacy and Lysosomal Stability of LRRK2 G2019S Targeting Oligonucleotides

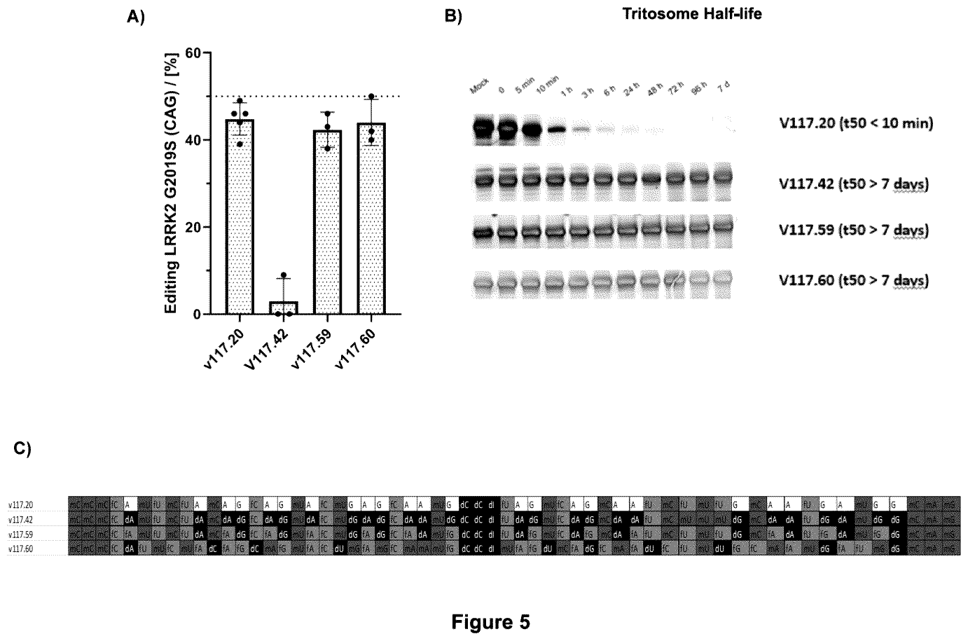

Several mutations in leucine-rich repeat kinase-2 (LRRK2) have been associated with Parkinson's disease (PD) with the G2019S as a very prominent one. To determine the impact of 2′-F, 2′-OMe- and 2′-H-backbone modifications on LRRK2 G2019S targeting oligonucleotides, differently modified constructs were generated and tested for their in vitro editing efficacy and lysosomal stability. The different human LRRK2 G2019S targeting constructs used in Example 5 are listed in Table 5. The results are shown in FIG. 5.

To assess cellular efficacy, 5×10⁴ HeLa cells were seeded in a 24-well plate. After 24 h, cells were forward transfected with 300 ng plasmid containing the human LRRK2 G2019S mutated cDNA and 0.9 μl FuGENE® 6. 24 h after plasmid transfection, 25 pmol of each construct was forward transfected with 1.5 μl Lipofectamine RNAiMAX reagent. After 24 h of incubation, cells were harvested for RNA isolation and Sanger sequencing. Lysosomal degradation assays were performed as described in Example 1.

TABLE 5
LRRK2 G2019S targeting construct sequences and modifications. mN = 2′-
O-methyl (2′-OMe), fN = 2′-fluoro (2′-F), N = 2′-OH (ribose; RNA), dN = 2′-H (deoxyribose;
DNA), * = phosphorothioate (PS) linkage.

| Construct | | Seq. | Fig. |
|---|---|---|---|
| Name | Construct Sequence (5′ to 3′ direction) | ID | No. |
| V117.20 | mC*mC*mC*fC*A*mU*fU*mC*fU*A*mC*A*G*fC*A*G*mU*A*fC*mU* | 49 | 5 |
| | G*A*G*fC*A*AmUG dCdCdI fUA G* mU* fC* A*G*mC* A* A* fU* | | |
| | mC* fU* mU* fU*G*mC*A*A*fU*G*A*mU*G*G*mC*mA*mG | | |
| V117.42 | mC*mC*mC*fC*dA*mU*fU*mC*fU*dA*mC*dA*dG*fC*dA*dG*mU* | 50 | 5 |
| | dA*fC*mU*dG*dA*dG*fC*dA*dA mUdG*dC*dC*dI fU dAdG*mU* fC* | | |
| | dA*dG* mC* dA*dA* fU* mC*mU* mU* mU* dG*mC* dA*dA*fU* | | |
| | dG*dA* mU* dG*dG*mC*mA*mG | | |
| V117.59 | mC*mC*mC*fC*fA*mU*fU*mC*fU*dA*mC*fA*dG*fC*fA*dG*mU*fA* | 51 | 5 |
| | fC*mU*dG*fA*dG*fC*fA*dAmUfG* dC*dC*dI fUdAfG*mU*fC*dA*fG* | | |
| | mC* dA*fA*fU*mC*fU* mU*fU*dG*mC*fA*dA*fU*fG*dA*mU* fG* | | |
| | dG*mC*mA*mG | | |
| V117.60 | mC*mC*mC*fC*dA*fU*mU*fC*mU*fA*dC*fA*fG*dC*mA*fG*mU*fA* | 52 | 5 |
| | fC*dU*mG*fA*mG*fC*mA*mAmUfG* dC*dC*dI mU fA fG* dU* mC* | | |
| | fA* dG* fC* mA* fA* du*fC*fU* mU* dU*fG*fC* mA* fA* mU* dG*fA* | | |
| | fU* mG* dG* mC*mA*mG | | |

As shown in FIG. 5A, backbone modifications comprising merely the replacement of 2′-OH groups in v117.20 with 2′-H groups in v117.42 (49.2% DNA, 16.9% 2′-F, 33.9% 2′-OMe) caused a strong decrease in editing efficacy (below 10% editing yield). However, oligonucleotides carrying a combination of 2′-F-, 2′-OMe-, and 2′-H modifications were able to restore their editing efficacy to that of the v117.20 construct. For example, v117.59, carrying a mixture of 42.4% 2′-F, 30.5% 2-OMe, and 27.1% 2′-H modifications had an editing efficacy around 40%, which was similar to that of v117.20. In fact, in addition to showing normal editing efficacy, v117.59 also showed enhanced lysosomal stability (t₅₀>7 days) (FIG. 5B). Similarly, v117.60, which comprises a mixture of 40.7% 2′-F, 37.3% 2-OMe, and 22% 2′-H modifications outside the CBT, not only showed stable editing efficacy but also experienced enhanced lysosomal stability (t₅₀>7 days) when compared to v117.20 (FIG. 5B).

These data confirm the observations made for SERPINA1 E342K (Example 2), STAT1 Y701 (Example 3) and CRB1 C948Y (Example 4) targeting constructs in that certain levels of 2′-F, 2′-OMe, and 2′-H modifications should be included in the oligonucleotide backbone in order to provide good editing efficacy and lysosomal stability. As already observed in Example 3, the length of uniform 2′-modification blocks does not exceed 6 nt, or even 4 nt. As observed previously in Example 2, embodiments containing high levels of DNA (nearly 50% for LRRK2 G2019S v117.42) combined with lower levels of 2′-F (just over 15% for LRRK2 G2019S v117.42) strongly enhance lysosomal stability but show compromised editing efficacy. Preferably, the LRRK2 G2019S targeting embodiments contain around 40% 2′-F, around 25% DNA and around 35% 2′-OMe. Also, these data suggest that this combination of 2′-modifications can be applied to different oligonucleotide sequences and thus used independently of the actual target (see, LRRK2 G2019S v117.60 and SERPINA1 E342K v117.59).

This demonstrates that the modification pattern may be transferred in a position-specific manner independent of the target sequences. While transferability between embodiments for SERPINA1 E342K and LRRK2 G2019S seems high, corresponding embodiments may still require some additional target-specific adaptations in order to provide ASOs with the optimal editing capacity.

### Example 6. Editing Efficacy of Murine PDE6A (mPDE6A) V685M Targeting Oligonucleotides

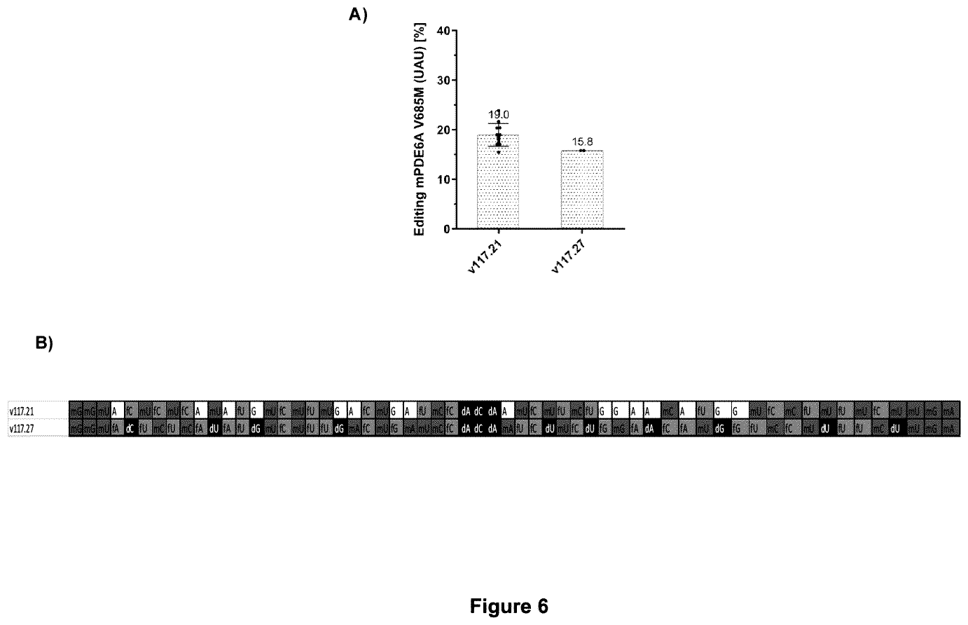

Mutations in the PDE6A gene can cause rod photoreceptors degeneration and have been associated with the blinding disease retinitis pigmentosa (RP) (Sothilingham et al., 2015). RP animal models include the Pde6a V685M mutant mouse model Therefore, to determine the effect of 2′-F, 2′-OMe, and 2′-H modifications on murine PDE6A (mPDE6A) targeting ASOs, a construct was prepared comprising such modifications and tested editing efficacy in vitro. The different constructs used in Example 6 are listed in Table 6. The results are shown in FIG. 6A.

TABLE 6
mPDE6A targeting construct sequences and modifications used in Example
6. mN = 2′-O-methyl (2′-OMe), fN = 2′-fluoro (2′-F), N = 2′-OH (ribose; RNA), dN = 2′-H
(deoxyribose; DNA), * = phosphorothioate (PS) linkage.

| Construct | | Seq. | Fig. |
|---|---|---|---|
| Name | Construct Sequence (5′ to 3′ direction) | ID | No. |
| V117.21 | C6-Aminolinker - | 53 | 6 |
| | mG* mG*mU*A*fC*mU*fC*mU* fC*A*mU*A*fU*G* mU* fC*mU*fU* mU* | | |
| | G*A*fC*mU*G*A*fU mC fC dAdCdA A mUfC* mU* fU* mC* fU* G*G* | | |
| | A*A*mC*A*fU*G*G*mU*fC*mC*fU*mU*fU*mU*fC* mU* mU* mG* mA | | |
| V117.27 | mG*mG*mU*fA*dC*fU*mC*fU*mC*fA*dU*fA*fU*dG*mU*fC*mU*fU*fU* | 54 | 6 |
| | dG*mA*fC*mU*fG*mA*mU mC fC* dA*dC*dA mA fU fC* dU*mU* fC* | | |
| | dU* fG* mG* fA* dA*fC* fA* mU* dG*fG* fU* mC* fC* mU* du*fU* fU* | | |
| | mC* dU*mU*mG*mA | | |

Construct v117.21 comprises a mixture of 2′-F-, 2′-OMe, and 2′-OH (RNA) (28.8% 2′F, 39% 2′OMe and 27.1% 2′OH nucleoside modifications. The results show a stable editing efficacy of around 19%. Replacing 2′-OH groups with 2-H groups and/or introducing 2′-F- and 2′-OMe-modifications gave rise to v117.27 (40.7% 2′F, 37.3% 2′OMe and 22% 2′H), which showed a similar editing efficacy.

These data confirm the previous observations in that a combination of 2′-F and 2′-OMe modifications together with 2′H groups can provide stable ASOs without negatively impacting their editing efficacy. As already observed in previous examples, the length of uniform 2′-OH modification blocks does not exceed 6 nt, or even 3 nt.

### Example 7. Editing Efficacy of NLRP3 Y166 Targeting Oligonucleotides

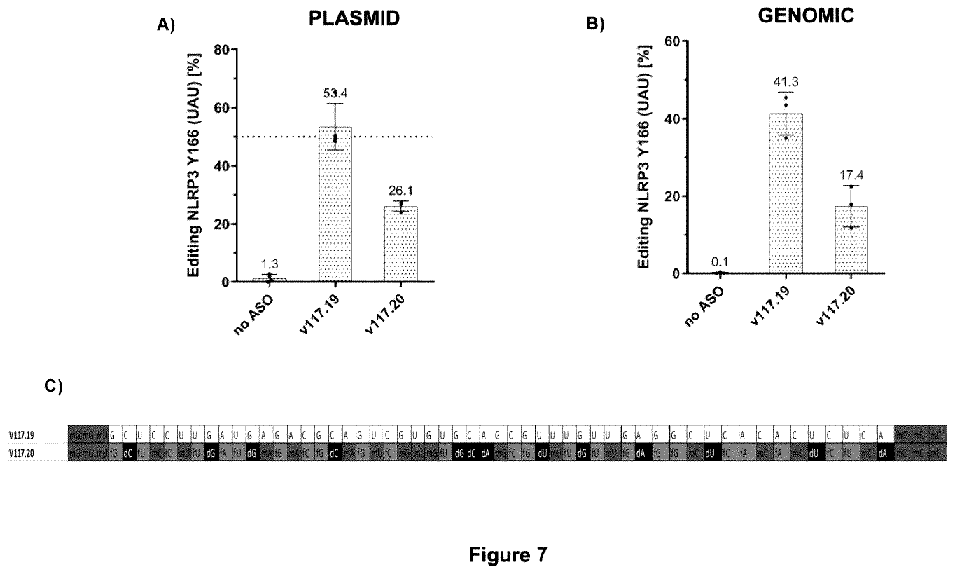

NLRP3 (nucleotide-binding domain, leucine-rich-repeat containing family, pyrin domain containing-3) inflammasome, has been reported to play a critical role in retinal neurodegeneration and many other diseases. NLRP3 is responsible for inflammasome formation, which is linked to several ailments such as inflammation, ageing, heart and vascular disease, metabolic syndrome, gout, autoimmune diseases, etc. It is often a factor that, once activated, prolongs the duration of sicknesses. The target site, Y166, is a phosphorylation site that is essential for NLRP3 activation (Bittner et al., 2021). Through beneficial RNA editing, the phosphorylation site can be blocked so that NLRP3 can no longer or less efficiently be activated. Accordingly, the editing efficacy of human NLRP3 Y166 targeting oligonucleotides was tested using a plasmid expression system (A) and a genomically integrated oligonucleotide sequence (B). The different NLRP3 Y166 targeting construct sequences and modifications used in Example 7 are shown in Table 7. The results are shown in FIG. 7. “no ASO” was used as negative control.

Plasmid-based approach (A): 5×10⁴ HeLa cells (Cat. No.: ATCC CCL-2) were seeded into a 24-well, before being transfected with 300 ng plasmid-containing wildtype NLRP3 cDNA and 0.9 μl FuGENE® 6 (Promega). 24 h post transfection, 25 pmol of the construct was forward transfected with 1.5 μl Lipofectamine RNAiMAX reagent. After 24 h incubation at 37° C., cells were harvested for RNA isolation and Sanger sequencing.

Genomically integrated approach (B): 1×10⁵ HeLa cells, containing human wildtype NLRP3-mNeonGreen cDNA stably integrated into their genome via the piggyBac transposase system, were seeded in a 24-well plate. 24 h post seeding, each well was forward transfected with 25 pmol construct and 1.5 μl RNAiMAX. After 24 h of incubation at 37° C., cells were harvested for RNA isolation and Sanger sequencing.

TABLE 7
NLRP3 Y166 targeting construct sequences and modifications used in
Example 7. mN = 2′-O-methyl (2′-OMe), fN = 2′-fluoro (2′-F), N = 2′-OH (ribose; RNA),
dN = 2′-H (deoxyribose; DNA), * = phosphorothioate (PS) linkage.

| Construct | | Seq. | Fig. |
|---|---|---|---|
| Name | Construct Sequence (5′ to 3′ direction) | ID | No. |
| V117.19 | mG*mG*mU*G*C*U*C*C*U*U*G*A*U*G*A*G*A*C*G*C*A*G*U*C*G* | 55 | 7 |
| | UGU GCA GCG* U* U* U* G*U*U* G* A* G* G* C* U* C* A* C* A* | | |
| | C*U* C*U*C*A*mC*mC*mC | | |
| V117.20 | mG*mG*mU*fG*dC*fU*mC*fC*mU*fU*dG*fA*fU*dG*mA*fG*mA*fC* | 56 | 7 |
| | fG*dC*mA*fG*mU*fC*mG* mUmGfU* dG*dC*dA mGfCfG* dU* mU* | | |
| | fU* dG*fU* mU* fG* dA*fG* fG* mC* du*fC* fA* mC* fA* mC* dU* fC* | | |
| | fU* mC*dA*mC*mC*mC | | |

As shown in FIGS. 7A and B, no significant difference was observed between the plasmid-based approach (A) and the genomically integrated approach (B). Overall, the data show that a combination of 40.7% 2′-F, 37.3% 2′-OMe and 22% 2′-H modifications was able to stabilise and preserve efficient editing yields (v117.20) and that this stabilisation and preservation was obtained without continuous (≤6 nt) blocks of 2′-modifications (neither 2′-F, nor 2′-O-methyl, nor DNA).

### Example 8. Adaptation of 5′ and 3′ Termini Lengths of GAPDH 3′UTR Targeting Oligonucleotides

Depending on the type and/or sequence of a particular ASO, and without being bound by any theory, the inventors believe that there is an optimal ASO length, where hybridization strength is optimal and ADAR harnessing is at least sufficient for editing. Furthermore, it is assumed that the shorter the ASO, the easier the delivery (e.g., endosomal escape), the lower the risk for aggregation, toxicity, and/or immunogenicity, and the easier it is to manufacture and screen such ASO on the large scale. Using the artificial SNAP-ADAR approach, the inventors have previously shown that the guide RNA can easily be shorten down to 14 nt, while still maintaining recognition of the substrate duplex and editing by ADAR's deaminase domain (data not shown). These latter findings differ from the prior art, where recruitment of endogenous ADAR to endogenous substrates was mediated by block design ASOs having stereopure PS/PN-modified backbones and a length of 30-33 nt (Monian et al., 2022).

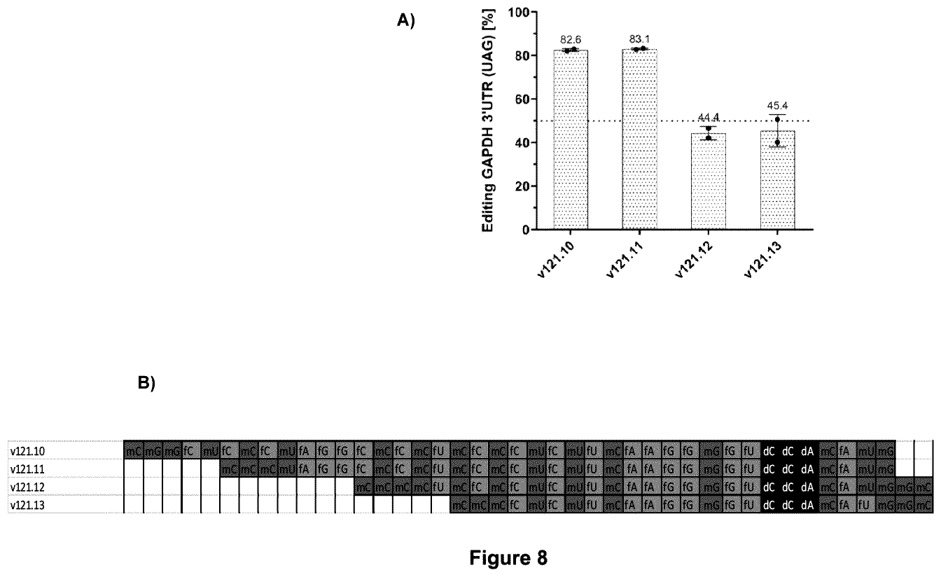

To determine the possible range of lengths for ADAR-recruiting oligonucleotides, four embodiments targeting the 3′UTR of human GAPDH were assessed for their editing efficacy in vitro (see, v121.10 to v121.13). The corresponding results of Example 8 are shown in FIG. 8. The different ASO constructs, and their modifications, are listed in Table 8.

To assess cellular efficacy, 1×10⁵ HeLa cells were forward transfected with 25 pmol construct and 1.5 μl Lipofectamine RNAiMAX Reagent (ThermoFisher Scientific) per 24-well according to the Lipofectamine RNAiMAX reagent protocol. The RNA was isolated 24 h later for Sanger sequencing.

TABLE 8
GAPDH 3′UTR targeting construct sequences and modifications used in
Example 8. mN = 2′-O-methyl, fN = 2′-fluoro, N = 2′OH (ribose; RNA), dN = 2′H
(deoxyribose; DNA), * = phosphorothioate linkage.

| Construct | | Seq. | Fig. |
|---|---|---|---|
| Name | Construct Sequence (5′ to 3′ direction) | ID | No. |
| V121.10 | mC*mG*mG*fC*mU*fC*mC*fC*mU*fA*fG*fG*fC*mC*fC*mC*fU* | 57 | 8 |
| | mC*fC*mC*fC*mU*fC*mU*fU*mC*fA*fA*fG*fG* mGfGfU | | |
| | dCdCdA *mC*fA*mU*mG | | |
| V121.11 | mC*mC*mC*mU*fA*fG*fG*fC*mC*fC*mC*fU*mC*fC*mC*fC*mU* | 58 | 8 |
| | fC*mU*fU*mC* fA*fA*fG*fG* mGfGfU dCdCdA *mC*fA*mU*mG | | |
| V121.12 | mC*mC*mC*mC*fU*mC*fC*mC*fC*mU*fC*mU*fU*mC* | 59 | 8 |
| | fA*fA*fG*fG* mGfGfU dCdCdA mCfAfU *mG*mG*mC | | |
| V121.13 | mC*mC*mC*fC*mU*fC*mU*fU*mC*fA*fA*fG*fG* mGfGfU | 60 | 8 |
| | dCdCdA mCfAfU *mG*mG*mC | | |

Overall, the data set shows that even very short embodiments (e.g., down to at least 25 nt) can achieve efficient editing.

### Example 9. Adaptation of the 5′ and 3′ Termini Lengths of SERPINA1 E342K Targeting Oligonucleotides

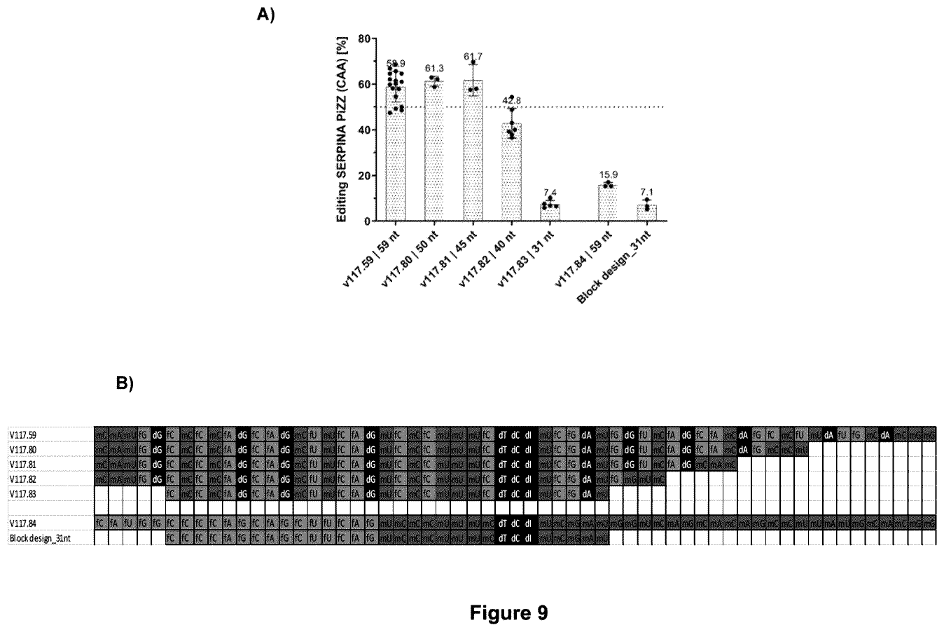

To determine the significance and role of the 5′- and 3′-terminal ends in RNA editing, several truncated versions of human SERPINA1 E342K targeting oligonucleotides were generated and assessed for their editing efficacy in vitro (see, v117.80 to v117.83). The corresponding results of Example 9 are shown in FIG. 9. The various 5′ and/or 3′ terminus truncated ASO constructs, and their modifications, are listed in Table 9. A 31 nt long oligonucleotide [Block design_31 nt] and a 40 nt long oligonucleotide [Block design_40 nt] served as negative controls.

TABLE 9
SERPINA1 E342K targeting construct sequences and modifications used in
Example 9. mN = 2′-O-methyl (2′-OMe), fN = 2′-fluoro (2′-F), N = 2′-OH (ribose; RNA),
dN = 2′-H (deoxyribose; DNA), * = phosphorothioate (PS) linkage.

| Construct | | Seq. | Fig. |
|---|---|---|---|
| Name | Construct Sequence (5′ to 3′ direction) | ID | No. |
| V117.59 | mC*mA*mU*fG*dG*fC*mC*fC*mC*fA*dG*fC*fA*dG*mC*fU*mU*fC | 20 | 9 |
| | *fA*dG*mU*fC*mC*fC*mU*mUmUfC* dT*dC*dImU fC fG* dA* | | |
| | mU* fG* dG*fU* mC* fA* dG*fC* fA* mC*dA*fG* fC* mC* fU* mU* | | |
| | dA*fU*fG*mC*dA*mC*mG*mG | | |
| V117.80 | mC*mA*mU*fG*dG*fC*mC*fC*mC*fA*dG*fC*fA*dG*mC*fU*mU*fC | 61 | 9 |
| | *fA*dG*mU*fC*mC*fC*mU*mUmUfC* dT*dC*dImU fC fG* dA* | | |
| | mU* fG*dG*fU*mC*fA*dG*fC*fA*mC*dA*fG*mC*mC*mU | | |
| V117.81 | mC*mA*mU*fG*dG*fC*mC*fC*mC*fA*dG*fC*fA*dG*mC*fU*mU*fC | 62 | 9 |
| | *fA*dG*mU*fC*mC*fC*mU*mUmUfC* dT*dC*dI mU fC fG* | | |
| | dA*mU*fG*dG*fU*mC*fA*dG*mC*mA*mC | | |
| V117.82 | mC*mA*mU*fG*dG*fC*mC*fC*mC*fA*dG*fC*fA*dG*mC*fU*mU* | 63 | 9, 10 |
| | fC*fA*dG*mU*fC*mC*fC*mU*mUmUfC* dT*dC*dI mU fC fG* dA* | | |
| | mU*fG*mG*mU*mC | | |
| V117.83 | fC*mC*fC*mC*fA*dG*fC*fA*dG*mC*fU*mU*fC*fA*dG*mU*fC*mC* | 64 | 9, 10 |
| | fC*mU*mUmUfC* dT*dC*dImUfCfG*dA*mU | | |
| V117.84 | fC*fA*fU*fG*fG*fC*fC*fC*fC*fA*fG*fC*fA*fG*fC*fU*fU*fC*fA*fG*mU | 65 | 9 |
| | *mC*mC*mC*mU*mU*mU*mC* dT*dC*dI*mU* mC* mG* mA* | | |
| | mU* mG* mG*mU*mC*mA*mG*mC*mA*mC*mA*mG* mC* mC* | | |
| | mU*mU* mA* mU*mG*mC*mA*mC*mG*mG | | |
| V117.85 | mC*mA*mU*fG*dG* fC* mC* fC* mC* fA* dG* fC*fA* dG*mC* fU* | 66 | 10 |
| | mU* fC*fA*dG*mU*fC*mC*fC*mU*mUmUfC* dT*dC*dI mU fC fG* | | |
| | dA*mU* fG*mG | | |
| V117.86 | mC*mA*mU*fG*dG* fC*mC* fC* mC* fA* dG* fC* fA* dG*mC* fU* | 67 | 10 |
| | mU* fC*fA*dG*mU*fC*mC*fC*mU*mUmUfC* | | |
| | dT*dC*dImUfCfG*dA*mU | | |
| V117.87 | mU*fG*dG*fC*mC*fC*mC*fA*dG*fC*fA*dG*mC*fU*mU*fC*fA*dG* | 68 | 10 |
| | mU*fC*mC*fC*mU*mUmUfC* dT*dC*dImUfCfG*dA*mU* fG* mG* | | |
| | mU*mC | | |
| V117.88 | fC*mC*fC*mC*fA*dG*fC*fA*dG*mC*fU*mU*fC*fA*dG*mU*fC*mC* | 69 | 10 |
| | fC*mU*mUmUfC* dT*dC*dI mU fC fG* dA*mU* fG* mG* mU* mC | | |
| V117.89 | mA*mU*fG*dG*fC*mC*fC*mC*fA*dG*fC*fA*dG*mC*fU*mU*fC*fA* | 70 | 10 |
| | dG*mU*fC*mC*fC*mU*mUmUfC* dT*dC*dImU fC fG* dA*mU* fG* | | |
| | mG* mU | | |
| V117.90 | mU*fG*dG*fC*mC*fC*mC*fA*dG*fC*fA*dG*mC*fU*mU*fC*fA*dG* | 71 | 10 |
| | mU*fC*mC*fC*mU*mUmUfC* dT*dC*dImUfCfG*dA*mU*fG*mG | | |
| V117.91 | fG*dG*fC*mC*fC*mC*fA*dG*fC*fA*dG*mC*fU*mU*fC*fA*dG*mU* | 72 | 10 |
| | fC*mC*fC*mU*mUmUfC* dT*dC*dImUfCfG*dA*mU*fG | | |
| V117.141 | fG*dG*fC*mC*fC*mC*fA*dG*fC*fA*dG*mC*fU*mU*fC*fA*dG*mU* | 73 | 10 |
| | fC*mC*fC*mU*mUmUfC* dT*dC*dImUfC*fG*dA*mU | | |
| V117.142 | dG*fC*mC*fC*mC*fA*dG*fC*fA*dG*mC*fU*mU*fC*fA*dG*mU*fC* | 74 | 10 |
| | mC*fC*mU*mUmUfC* dT*dC*dImUfC*fG*dA*mU | | |
| Block | fC*fC*fC*fC*fA*fG*fC*fA*fG*fC*fU*fU*fC*fA*fG*MU*mC*mC*mC* | 75 | 9, 10 |
| design_31 nt | mU*mU*mU*mC* dT*dC*dI*mU*mC*mG*mA*mU | | |
| Block | fC*fA *fU*fG*fG*fC*fC*fC*fC*fA*fG*fC*fA*fG*fC*fU*fU*fC*fA*fG*mU | 76 | 10 |
| design_40 nt | *mC*mC*mC*mU*mUmUmC* dT*dC*dI mU mC mG* mA* mU* | | |
| | mG* mG* mU*mC | | |

Initially, different 3′ terminus truncated ASO constructs were tested for their ability to maintain RNA editing efficacy (FIG. 9). A symmetric 59 nt long ASO (v117.59) served as the starting point and was sequentially truncated from the 3′ terminus (while maintaining the 3′ terminal 1-3×2′-OMe modifications) to give rise to 3′ terminus truncated ASOs of varying lengths (v117.80 to v117.83) (FIG. 9B). As shown in (FIG. 9A), 3′-end shortening of v117.59 to 50 nt (v117.80) or to 45 nt (v117.81) did not change the editing yield. While experiencing a slight decrease, construct v117.82 (40 nt) maintained its ability to efficiently mediate SERPINA target editing. Most significantly, constructs v117.81 and v117.80 even showed a small increase in editing efficacy when compared to v117.59 (FIG. 9A).

However, simultaneous truncation of the 5′ and 3′ termini led to a significant reduction in editing efficacy close to the detection limit with Sanger sequencing (v117.83; 31 nt) (FIG. 9A). That is, while 3′ terminus shortening to 40 nt resulted in only a slight reduction in editing efficacy, shortening of the ASO from the 3′ terminus, and in particular, the 5′ terminus, down to 31 nt (v117.83) nearly led to a complete loss in editing (FIG. 9A). Without being bound by any theory, the inventors submit that truncation from the 3′ terminus may be better tolerated than from the 5′ terminus.

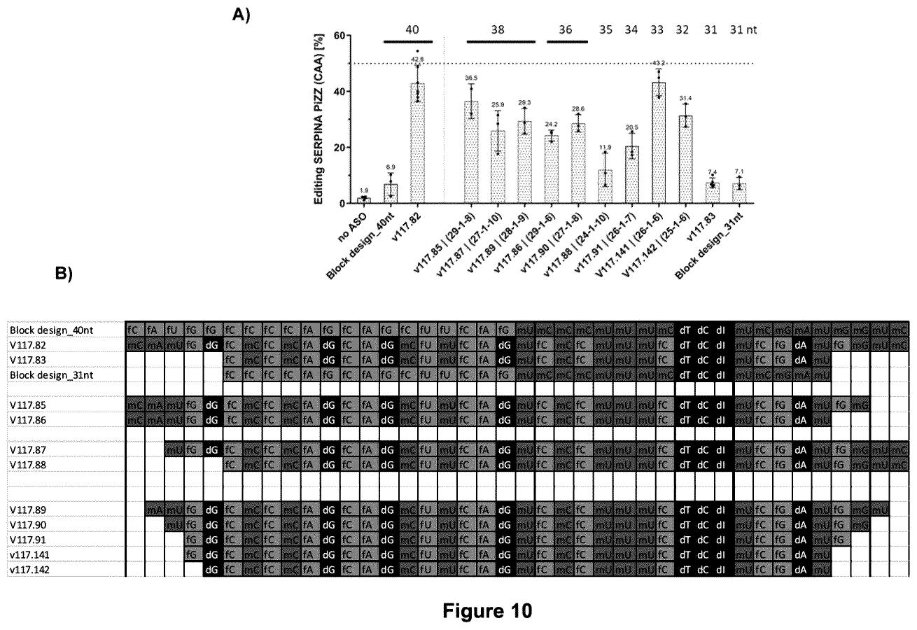

As a next step, and to further determine the importance of the 5′ and 3′ termini in regulating editing efficacy, additional constructs carrying different combinations of 5′ and 3′ termini truncations were tested. Specifically, to assess the editing efficacy of 5′ and 3′ terminus double truncated ASOs, different ASOs were generated lacking 1, 2, 3, 4 or 5 nucleobases at one or both termini. “no ASO” served as negative control. SERPINA1 E342K targeting constructs and their modifications are also listed in Table 9. The results are shown in FIG. 10.

ASOs carrying 5′ and 3′ terminus truncations (v117.83 and a block design_31 nt) showed an overall decrease in editing efficacy when compared to full length construct (v117.82 and a Block design_40 nt) (FIG. 10A). Interestingly, two constructs (v117.85 and v117.86) with a 3′ terminus truncation only showed a small decrease when compared to the full-length construct (v117.82), indicating that 3′ terminus truncations are generally well tolerated, particularly to 5 nt length outside of the CBT. On the other hand, 5′ terminus truncations had a greater impact on the editing efficacy of the particular oligonucleotides (v117.87 and v117.88). Surprisingly, the truncation of the 5′ terminus to only 25 or 24 nucleotides outside of the CBT was particularly effective, especially when combined with a 3′ terminus length of 5 nucleotides, yielding ASOs v117.141 and v117.142 (of 33 and 32 nt length, respectively). As shown in FIG. 10, 5′ and 3′ terminus truncated versions maintained an editing efficacy similar to that of 3′ truncated ASOs.

Overall, these data confirm the results presented above, in that a combination of 2′-F and 2′-OMe modifications together with 2′H groups is able to provide stable ASOs without negatively impacting editing efficacy. The observed ranges of 2′-F modifications lie between 37.5-42.4% (v117.141 and v117.82, respectively), the range for 2′H lies between 20-25% (v117.82 and v117.142, respectively) and the range for 2′-OMe lies between 32.4% and 40% (v117.91 and v117.88, respectively). Importantly, the data confirm that blocks of continuous sugar modifications should be avoided, and certain levels of 2′-F and 2′-OMe modifications included to obtain efficient editing yields, as all embodiments contain uniform 2′-modification blocks that do not exceed 6 nt, or even 3 nt. Furthermore, the data of Example 9 demonstrate that truncation of the 3′ terminus is generally better tolerated than truncation of the 5′ terminus. Conclusively, truncation of the oligonucleotide results in an asymmetric positioning of the oligonucleotide around the CBT, for which there are preferred specific 5′ and 3′ termini lengths. That is, shortening of the 3′ terminus is tolerated to a length of about 5 nt 3′ of the CBT (e.g., v117.86, v117.141, v117.142), while 5′ terminus shortening strongly decreases the overall editing efficacy of the oligonucleotides (e.g., v117.88). Surprisingly, the data from Example 9 shows that when optimal 2′-modifications, and ideal internucleoside modification/PS pattern and correct truncations are applied to the 5′ and 3′ terminus, very short (33 and 32 nt) and highly effective oligonucleotides can be obtained. This was the case in particular for the symmetries 5′-24-3-5 (scheme: 5′ terminus-CBT-3′ terminus) for 32 nt embodiments (v117.142), and 5′-25-3-5 for 33 nt embodiments (v117.141). Lastly, the data suggests that a high content of stereopure linkage modifications is not necessarily required to obtain high editing yields with short and stable ASOs.

### Example 10. Fine Screening of Truncated SERPINA (33 nt/32 nt) Targeting Oligonucleotides

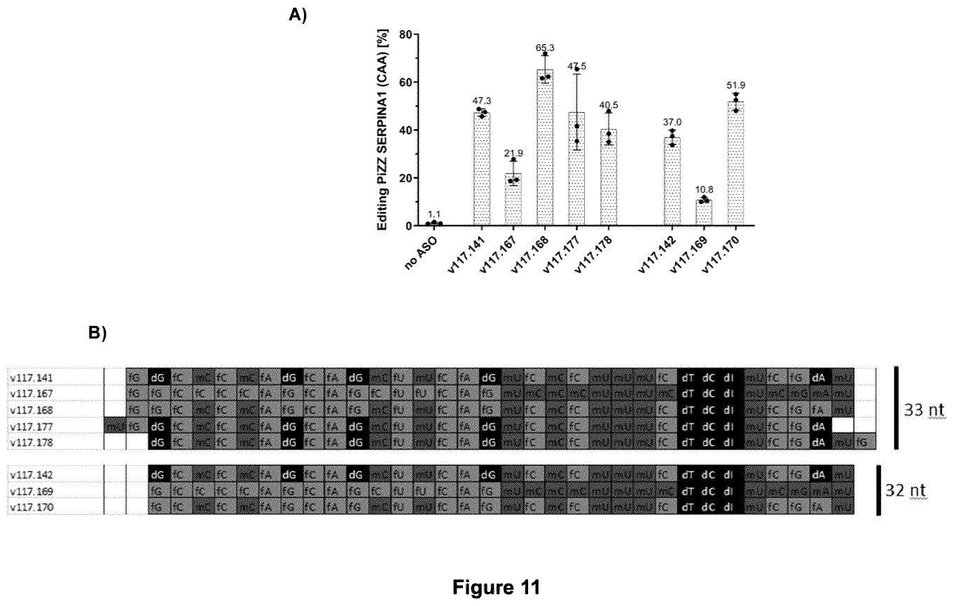

To determine the joint effect of continuous and/or interrupted 2′-F- and 2′-OMe-modifications in combination with DNA on the editing efficacy of short (32 or 33 nt), asymmetric ASOs, several SERPINA targeting constructs were prepared and used in additional screening experiments. The different 32 and 33 nt ASO designs and respective backbone modifications are listed in Table 10. The corresponding results are shown in FIG. 11. “no ASO” was used as negative control.

TABLE 10
32 and 33 nt long SERPINA1 targeting construct sequences and
modifications used in Example 10. mN = 2′-O-methyl (2′-OMe), fN = 2′-fluoro (2′-F), N =
2′-OH (ribose; RNA), dN = 2′-H (deoxyribose; DNA), * = phosphorothioate (PS) linkage.

| Construct | | Seq. | Fig. |
|---|---|---|---|
| Name | Construct Sequence (5′ to 3′ direction) | ID | No. |
| V117.141 | fG*dG*fC*mC*fC*mC*fA*dG*fC*fA*dG*mC*fU*mU*fC*fA*dG*mU* | 73 | 11 |
| | fC*mC*fC*mU*mUmUfC* dT*dC*dImUfC*fG*dA*mU | | |
| V117.167 | fG*fG*fC*fC*fC*fC*fA*fG*fC*fA*fG*fC*fU*fU*fC*fA*fG*mU*mC*mC* | 77 | 11 |
| | mC*mU*mUmUmC* dT*dC*dImUmC*mG*mA*mU | | |
| V117.168 | fG*fG*fC*mC*fC*mC*fA*fG*fC*fA*fG*mC*fU*mU*fC*fA*fG*mU*fC* | 78 | 11 |
| | mC*fC*mU*mUmUfC* dT*dC*dImU fC* fG*fA*mU | | |
| V117.177 | mU*fG*dG*fC*mC*fC*mC*fA*dG*fC*fA*dG*mC*fU*mU*fC*fA*dG* | 79 | 11 |
| | mU*fC*mC*fC*mU*mUmUfC* dT*dC*dImUfC*fG*dA | | |
| V117.178 | dG*fC*mC*fC*mC*fA*dG*fC*fA*dG*mC*fU*mU*fC*fA*dG*mU*fC* | 80 | 11 |
| | mC*fC*mU*mUmUfC* dT*dC*dImU fC*fG*dA*mU*fG | | |
| V117.142 | dG*fC*mC*fC*mC*fA*dG*fC*fA*dG*mC*fU*mU*fC*fA*dG*mU*fC* | 74 | 11 |
| | mC*fC*mU*mUmUfC* dT*dC*dImU fC*fG*dA*mU | | |
| V117.169 | fG*fC*fC*fC*fC*fA*fG*fC*fA*fG*fC*fU*fU*fC*fA*fG*mU*mC*mC*mC | 81 | 11 |
| | *mU*mUmUmC* dT*dC*dImU mC*mG*mA*mU | | |
| V117.170 | fG*fC*mC*fC*mC*fA*fG*fC*fA*fG*mC*fU*mU*fC*fA*fG*mU*fC*mC | 82 | 11 |
| | *fC*mU*mUmUfC* dT*dC*dImU fC*fG*fA*mU | | |

As shown in FIG. 11A, a 33 nt long construct (v117.141), containing a mixture of 42.4% 2′-F- and 33.3% 2′-OMe-modifications, and 24.2% DNA (5 nt outside the CBT) showed good editing (47.3%) when compared to v117.167, a construct based on the prior art that contains continuous blocks of 2′-F-modifications at the 5′ terminus and 2′-OMe-modifications 5′ and 3′ to the CBT (blocks of ≤5 nt) (FIG. 11B). Interestingly, blocks of 2′-F- and 2′-OMe modifications of limited size (e.g., ≤5 nt) were very well tolerated (v117.168) in the 33 nt constructs.

Similarly, for the 32 nt long constructs, disruption of continuous 2′-F-stretches at the 5′ end by 2′-OMe-modifications and DNA led to an increase in overall editing efficacy (see, v117.169 vs v117.142 and v117.170) (FIG. 11A). Versions v117.168 and v117.170 both showed that the exchange of DNA from the short (<40 nt) versions by 2′F can increase editing yields. Specifically, the increase in editing efficacy seen with v117.170 suggests that blocks of continuous 2′-F- and 2′-OMe modifications with limited size (e.g., ≤6 nt) may be beneficial in 33 nt and 32 nt short ASOs. Furthermore, it shows that short ASOs might benefit from a reduced amount of DNA nucleosides (≤6 nt) outside the CBT (v117.141 versus v117.168 and v117.142 versus v117.170). Conclusively, this suggests that a higher 2′-F content (ranging between 40.6-57.6% in v117.142 and v117.168, respectively) is well tolerated and even preferable for short embodiments (≤50 nt).

Overall, these findings were similar to the editing efficacy reported for an oligonucleotide previously described (Monian et al., 2022). Without being bound by any theory, the inventors believe that for effective editing efficacy, ASOs with a length of 30 nt (or “sweet spot” of around 33/32 nt) (see, results in FIG. 11) are dependent on certain modifications rules, i.e., the absence of uniform blocks of 2′-F in the 5′-half (5′ to the CBT) and 2′-OMe in the 3′-half (3′ to the CBT) of the ASO. This was also shown in Example 9 for the 40 nt lead, where the uniform solution (i.e., consecutive 2′-F-modifications) was comparably inefficient in target editing.

Hence, the inventors believe that the editing efficacy and stability of oligonucleotides may be optimised by using a combination of chemical modifications. For example, a large amount of stereopure linkage modifications (or the mere introduction of stereopure linkages) may be possible upon using an optimal 2′- and stereorandom linkage modification pattern in an oligonucleotide with an optimised length and asymmetry. However, the data shows that efficient RNA editing is accessible with chemically modified oligonucleotides that do not contain large amounts of stereopure internucleoside linkages given that an optimal modification pattern (maximal block size, e.g., ≤6 nt) and amount of 2′-F and 2′-O-alkyl (approx. 35-65% 2′-F and 30-35% 2′-O-alkyl) and natural as well as stereorandom internucleoside modification pattern (e.g., linkage d, e, are modified; linkage h, and i are not phosphorothioate) is obtained. While this does not exclude that a small number stereopure linkage modifications might improve editing efficiency further (e.g., not more than 10, more preferably not more than 5), it clearly shows that the high levels of stereopure linkage modifications, as seen in the prior art, are not absolutely essential. Furthermore, the data shows that the positioning of the ASO, specifically the lengths of the asymmetric termini, comprising at least 4 nt 3′ the CBT, and/or at least 16 nt 5′ of the CBT, is very important in combination with the other modification rules to obtain very efficient editing oligonucleotides of short length (e.g., below 40 nt).

### Example 11. Adaptation of the 3′ Terminus Length of STAT1 Y701 Targeting Oligonucleotides

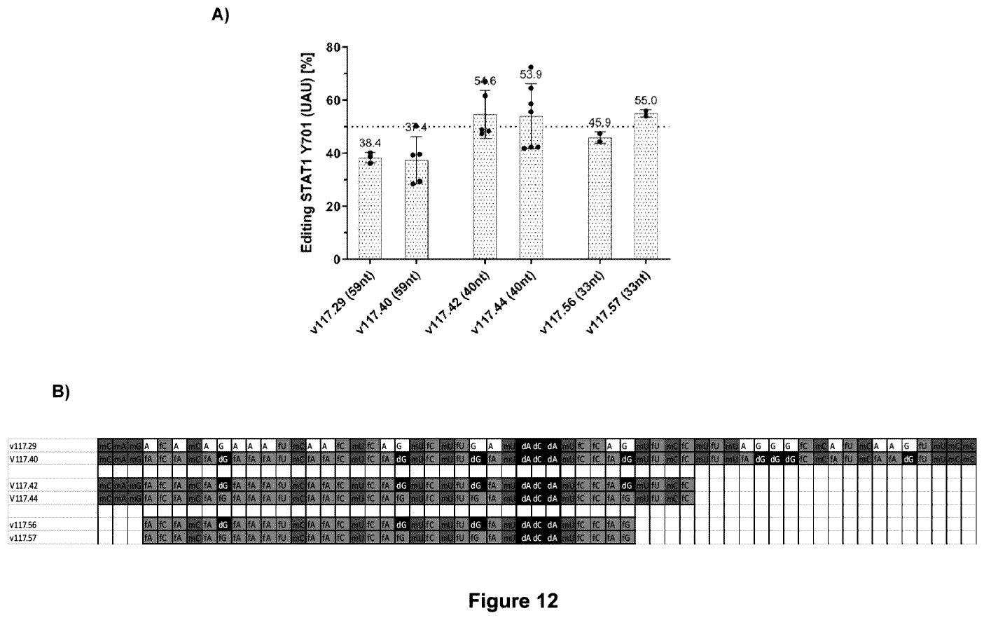

To determine the combined effect of 2′-F- and 2′-OMe-modifications, linkage modifications and DNA on editing efficacy, similar truncation experiments were conducted for STAT1 Y701 targeting oligonucleotides. The specific oligonucleotide construct sequences and their modifications used in Example 11 are listed in Table 12, while the corresponding results are shown in FIG. 12.

TABLE 12
STAT1 Y701 targeting construct sequences and modifications used in
Example 11. mN = 2′-O-methyl (2′-OMe), fN = 2′-fluoro (2′-F), N = 2′-OH (ribose; RNA),
dN = 2′-H (deoxyribose; DNA), * = phosphorothioate (PS) linkage, GalNAc = triantennary
N-acetylgalactosamine.

| Construct | | Seq. | \|Fig. |
|---|---|---|---|
| Name | Construct Sequence (5′ to 3′ direction) | ID | No. |
| V117.29 | mC*mA*mG*A*fC*A*mC*A*G*A*A*A*fU*mC*A*A*fC*mU*fC*A*G*mU* | 35 | 12 |
| | fC*mU*fU*GAmU* dA*dC*dA mUfC fC* A* G* mU* fU* mC* fC* mU* | | |
| | fU* mU* A*G*G*G*fC*mC*A*fU*mC*A*A*G*fU*mU*mC*mC - GalNAc | | |
| V117.40 | mC*mA*mG*fA*fC*fA*mC*fA*dG*fA*fA*fA*fU*mC*A*fA*fC*mU*fC*fA* | 42 | 12 |
| | dG*mU*fC*mU*fU*dGfAmU* dA*dC*dA mUfC fC* fA* dG* mU* fU* | | |
| | mC* fC* mU* fU* mU* fA* dG*dG*dG* fC* mC* fA* fU* mC* fA* fA* dG* | | |
| | fU*mU*mC*mC | | |
| V117.42 | mC*mA*mG*fA*fC*fA*mC*fA*dG*fA*fA*fA*fU*mC*fA*fA*fC*mU*fC*fA* | 83 | 12 |
| | dG* mU*fC*mU*fU*dGfAmU* dA*dC*dA mUfC fC* fA* dG*mU* fU* | | |
| | mC* fC | | |
| V117.44 | mC*mA*mG*fA*fC*fA*mC*fA*fG*fA*fA*fA*fU*mC*fA*fA*fC*mU*fC*fA* | 84 | 12 |
| | fG*mU*fC*mU*fU*fGfAmU* dA*dC*dA mUfC fC*fA*fG*mU*fU*mC*fC | | |
| V117.56 | fA*fC*fA*mC*fA*dG*fA*fA*fA*fU*mC*fA*fA*fC*mU*fC*fA*dG*mU*fC* | 85 | 12 |
| | mU*fU*dG fA mU* dA*dC*dA mUfC fC*fA*fG | | |
| V117.57 | fA *fC*fA*mC*fA*fG*fA*A*fA*U*mC*fA*fA*fC*mU*fC*fA*fG*mU*fC*mU* | 86 | 12 |
| | fU*fGfAmU* dA*dC*dA mUfC fC*fA*fG | | |

As basis for introducing a mixture of 2′-F, 2′-OMe and 2′-H backbone modifications and subsequently generating a 3′ terminus truncated ASO (v117.42), the 59 nt long ASO construct (v117.29) was used (cf. Example 3). Construct v117.29, where most linkages are phosphorothioate (PS) linkages and which does not contain any 2′-H modifications outside the CBT, had a lysosomal stability of 2 h (data not shown) and an editing efficacy of about 38%. This editing efficacy was slightly reduced to 37.4% in construct v117.40 (59 nt), which contains a mixture of 49.2% 2′-F, 32.2% 2′-OMe and 13.5% DNA (8 nt outside the CBT) modifications outside the CBT (FIG. 12A). Moreover, shortening of the 3′ terminus to achieve a total oligonucleotide length of 40 nt (v117.42 and v117.44) improved the editing yields compared to the efficacy of the parent construct v117.29, thereby shifting the 2′-OH modification ranges up to 62.5% 2′F (v117.44), down to 30% 2′O-Me and to 0% DNA outside of the CBT. The achieved editing yields (approx. 50%) are even maintained when the oligonucleotide is further shortened down to 33 nt (v117.56 and v117.57). These embodiments contain 60.6-69.7% 2′-F and 9.1-0% DNA (up to 3 nt DNA outside the CBT, v117.56 and v117.57, respectively). Particularly interesting is that these short embodiments (≤50 nt) also tolerate comparably low amounts of 2′-OMe modifications (21.2% in v117.56 and v117.57). This demonstrates that a full 2′ ribose modification through a proper pattern of 2′-F- and 2′-OMe-modifications, PS linkages and DNA is to be observed when a reasonable editing efficacy shall be achieved.

Generally, these findings suggest that a mixture of particular oligonucleotide modifications is needed to maintain, improve or restore ASO editing efficacy. Specifically, the data not only show that a mixture of 2′-F, 2′-OMe and 2′-H modifications can be used to stabilize or maintain the editing efficacy of the ASO, but also that a subsequent shortening of these oligonucleotides down to at least 33 nt embodiments, containing such mixtures of 2′-F, 2′-OMe and 2′-H modifications and PS linkages, does not negatively impact the overall editing efficacy, Accordingly, a mixture of 2′-F-, 2′-OMe- and 2′-H- as well as PS linkage modifications is needed when a reasonable editing efficacy ought to be achieved. Furthermore, this dataset presents embodiments that shows that block sizes of 2′-F up to 6 nt are well accepted and that embodiments of short ASO length (e.g., ≤below 45 nt) prefer slightly higher 2′-F content (e.g., up to 70%) and benefit from a reduced DNA content (down to 0 nt) outside the CBT.

### Example 12. Adaptation of the 3′ Terminus Length of mCTNNB1 T41 Targeting Oligonucleotides

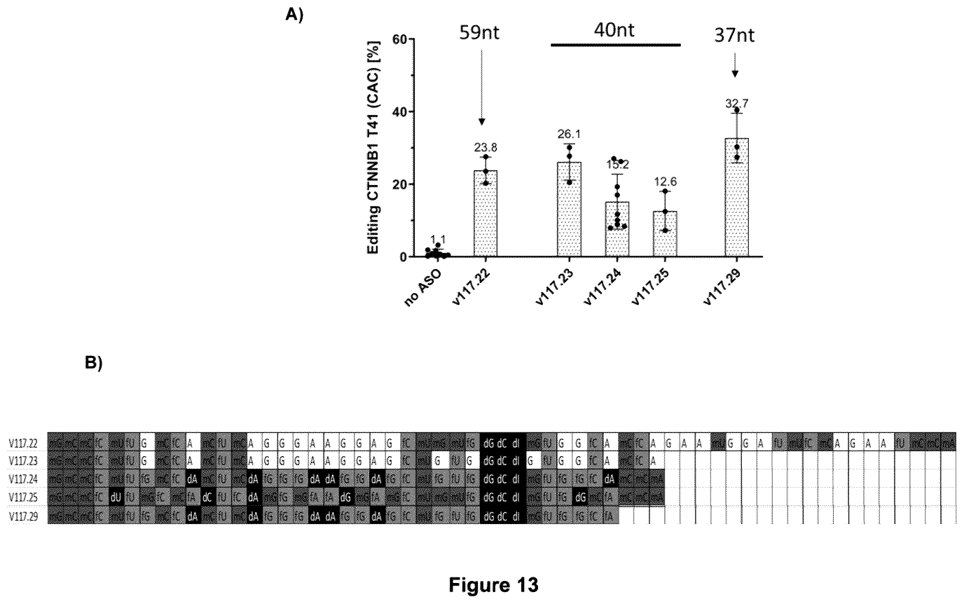

To further test the hypothesis that a certain, optimal mixture of 2′-F-, 2′-OMe- and 2′-H- as well as PS linkage modifications is required to provide stable and effective ASOs and to further validate the results obtained with 3′ terminally truncated STAT1 targeting ASOs (Example 10), similar truncation tests were conducted using mCTNNB1 T41 targeting oligonucleotides. The specific construct sequences and their modifications are listed in Table 12. Corresponding results are shown in FIG. 13. “no ASO” served as negative control.

TABLE 12
mCTNNB1 T41 targeting construct sequences and modifications used in
Example 12. mN = 2′-O-methyl (2′-OMe), fN = 2′-fluoro (2′-F), N = 2′-OH (ribose; RNA),
dN = 2′-H (deoxyribose; DNA), * = phosphorothioate (PS) linkage.

| Construct | | Seq. | Fig. |
|---|---|---|---|
| Name | Construct Sequence (5′ to 3′ direction) | ID | No. |
| V117.22 | mG*mC*mC*fC*mU*fU*G*mC*fC*A*mC*fU*mC*A*G*G*G*A*A*G*G* | 87 | 13 |
| | A*G*fC*mU* mGmUfG* dG*dC*dI mGfUG* G* fC* A* mC* fC* A* | | |
| | G*A*A*mU*G*G*A*fU*mU*fC*mC*A*G*A*A*fU*mC*mC*mA | | |
| V117.23 | mG*mC*mC*fC*mU*fU*G*mC*fC*A*mC*fU*mC*A*G*G*G*A*A*G*G* | 88 | 13 |
| | A*G*fC*mU* GfUG* dG*dC*dI GfUG* G*fC*A*mC*fC*A | | |
| V117.24 | mG*mC*mC*fC*mU*fU*fG*mC*fC*dA*mC*fU*mC*dA*fG*fG*fG*dA* | 89 | 13 |
| | dA*fG*fG*dA*fG*fC*mU* fGfUfG* dG*dC*dI mG fUfG* fG* fC* | | |
| | dA*mC*mC*mA | | |
| V117.25 | mG*mC*mC*fC*dU*fU*mG*fC*mC*fA*dC*fU*fC*dA*mG*fG*mG*fA*fA | 90 | 13 |
| | *dG*mG*fA*mG*fC*mU* mG mU fG* dG*dC*dI mGfUfG* | | |
| | dG*mC*fA*mC*mC*mA | | |
| V117.29 | mG*mC*mC*fC*mU*fU*fG*mC*fC*dA*mC*fU*mC*dA*fG*fG*fG*dA* | 91 | 13 |
| | dA*fG*fG*dA*fG*fC*mU* fG fU fG* dG*dC*dI mGfUfG* fG*fC*fA | | |

The different ASOs were based on v117.22, a 59 nt long construct comprising a mixture of 2′-F- and 2′-OMe-modifications, RNA/DNA and PS linkage modifications. On the basis of this construct, three 3′ terminus truncated versions comprising different mixtures of 2′-F- and 2′-OMe-modifications, RNA/DNA and PS linkage modifications were generated (FIG. 13). Replacement of all native RNA nucleosides with DNA and additional 2′-F- and 2′-OMe-modifications initially caused a drop in editing efficacy (v117.24, 15.2% and v117.25, 12.6%). Interestingly, the 37 nt long construct (v117.29), which comprised an overall DNA content of as high as 21.6% (5 nt outside the CBT), as well as 2′-F- and 2′-OMe-modifications showed an editing efficacy of 32.7%.

These data suggest that for 40 nt long ASOs, a mixture of 2′-F- and 2′-OMe-modifications, RNA/DNA content and PS linkage modifications is important for editing. Particularly for these embodiments, up to six DNA nucleosides were tolerated outside of the CBT for short (≤50 nt, here: 37-40 nt) oligonucleotides.

### Example 13. Adaptation of the 5′ and 3′ Termini Lengths of CRB1 C948Y Targeting Oligonucleotides

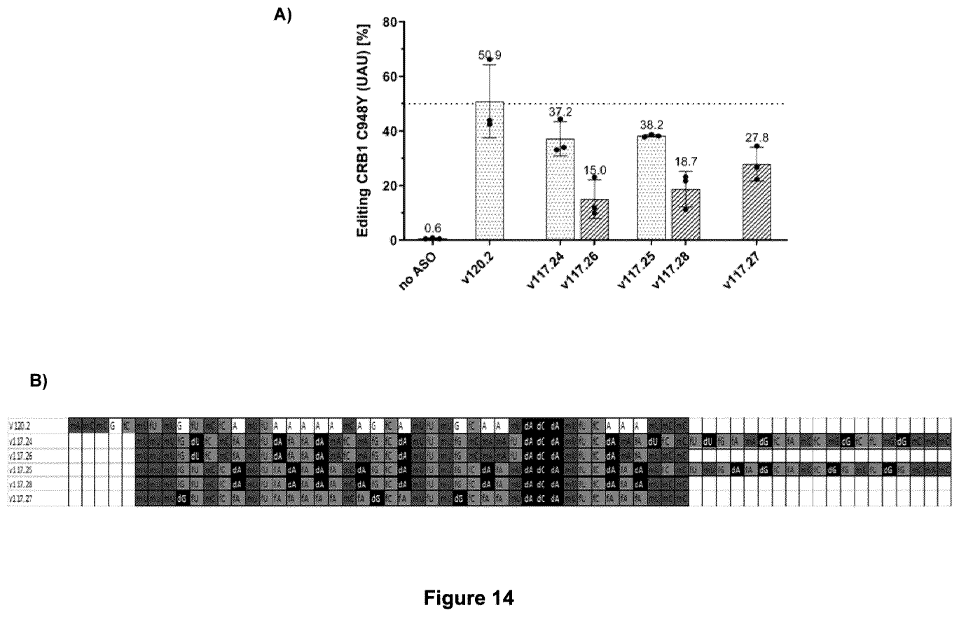

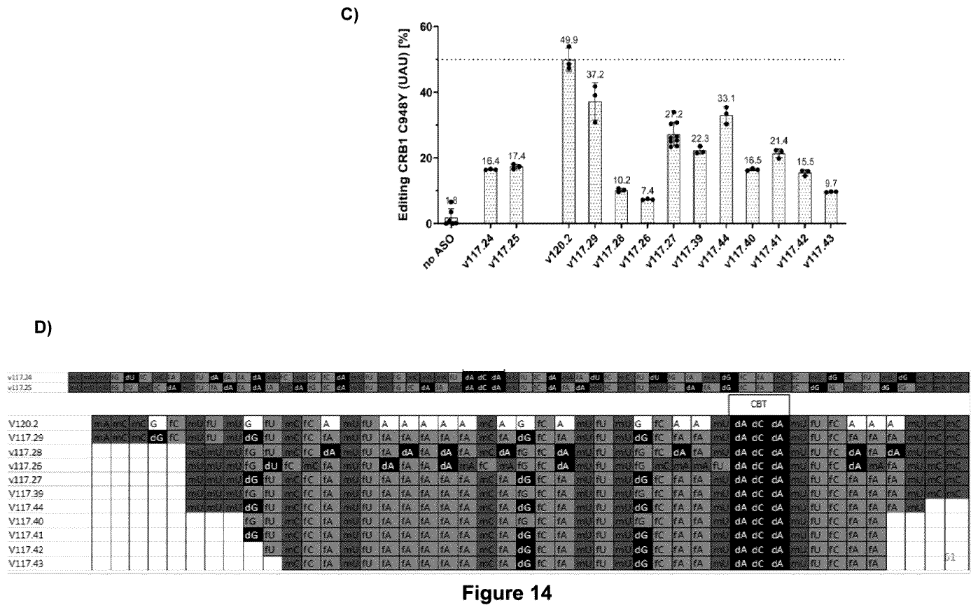

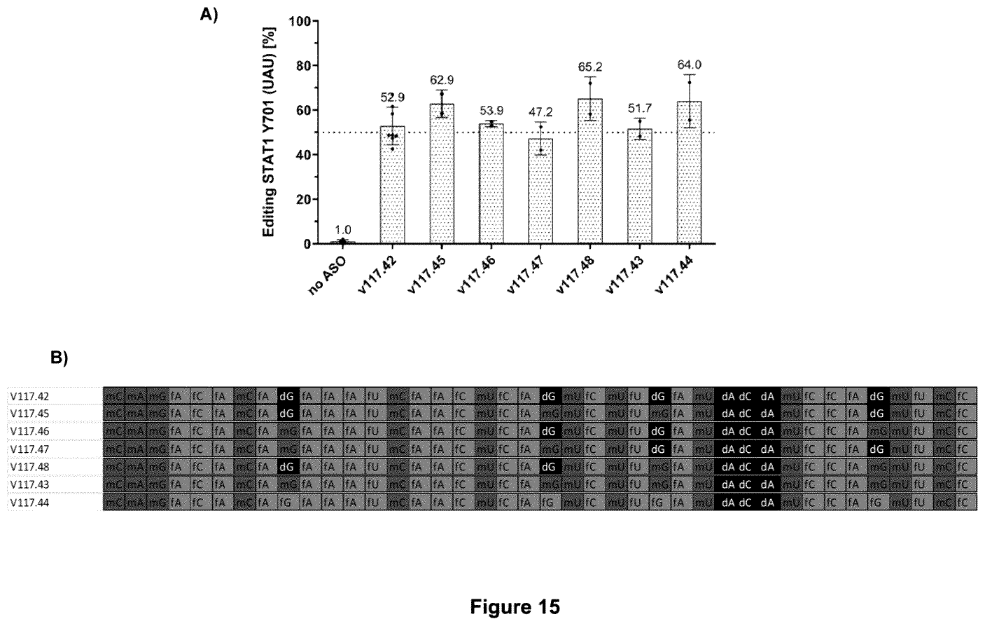

Analogous to the generation of 5′ and 3′ termini truncated constructs specific to the SERPINA1 target (Example 8), 5′ and 3′ terminally truncated versions of CRB1 C948Y specific oligonucleotides were generated and tested for their RNA editing efficacy in vitro. The different constructs tested in Example 13 are listed in Table 13. The results are shown in FIG. 14. “no ASO” served as negative control.

TABLE 13
CRB1 C948Y targeting construct sequences and modifications used in
Example 13. mN = 2′-O-methyl (2′-OMe), fN = 2′-fluoro (2′-F), N = 2′-OH (ribose; RNA),
dN = 2′-H (deoxyribose; DNA), * = phosphorothioate (PS) linkage.

| Construct | | Seq. | Fig. |
|---|---|---|---|
| Name | Construct Sequence (5′ to 3′ direction) | ID | No. |
| V120.2 | mA*mC*mC*G*fC*mU*fU*mU*G*fU*mC*fC*A*mU*fU*A*A*A*A*A*mC | 92 | 14 |
| | *A*G*fC*A*mU*fU*mU*G*fC* AAmU* dA*dC*dA | | |
| | mUfUfC*A*A*A*mU*mC*mC | | |
| V117.24 | mU*mU*mU*fG*dU*fC*mC*fA*mU*fU*dA*fA*fA*dA*mA*fC*mA*fG*fC | 47 | 14 |
| | *dA*mU*fU*mU*fG*mC* mAmAfU* dA*dC*dA mUfUfC | | |
| | *dA*mA*fA*dU*fC*mC*fU*dU*fG*fA*mA*dG*fC*fA*mC*fC*mG*dG*fC | | |
| | *fU*mG*dG*mC*mA*mC | | |
| V117.25 | mU*mU*mU*fG*fU*mC*fC*dA*mU*fU*fA*dA*fA*dA*fA*mC*dA*fG*fC* | 48 | 14 |
| | dA*mU*fU*mU*fG*fC* dAfAmU* dA*dC*dA mUfUfC *dA*fA* dA*mU* | | |
| | fC*mC*fU*mU*fG*dA*fA*dG*fC*fA*mC*fC*dG*fG*mC*fU*dG*fG*mC* | | |
| | mA*mC | | |
| V117.28 | mU*mU*mU*fG*fU*mC*fC*dA*mU*fU*fA*dA*fA*dA*fA*mC*dA*fG*fC* | 93 | 14 |
| | dA*mU*fU*mU*fG*fC* dAfAmU* dA*dC*dA mUfUfC *dA* fA* dA* | | |
| | mU* mC* mC | | |
| V117.26 | mU*mU*mU*fG*dU*fC*mC*fA*mU*fU*dA*fA*fA*dA*mA*fC*mA*fG*fC | 94 | 14 |
| | *dA*mU*fU*mU*fG*mC* mAmAfU* dA*dC*dA mUfUfC *dA*mA* fA* | | |
| | mU* mC* mC | | |
| V117.27 | mU*mU*mU*dG*fU*mC*fC*fA*mU*fU*fA*fA*fA*fA*fA*mC*fA*dG*fC*fA | 95 | 14 |
| | *mU*fU*mU*dG*fC* fAfAmU* dA*dC*dA mUfUfC | | |
| | *fA*fA*fA*mU*mC*mC | | |
| V117.29 | mA*mC*mC*dG*fC*mU*fU*mU*dG*fU*mC*fC*fA*mU*fU*fA*fA*fA*fA* | 96 | 14 |
| | fA*mC*fA*dG*fC*fA*mU*fU*mU*dG*fC* fAfAmU* dA*dC*dA mUfUfC | | |
| | *fA*A*fA*mU*mC*mC | | |
| V117.39 | C6 Aminolinker - | 97 | 14 |
| | mU*mU*mU*fG*fU*mC*fC*fA*mU*fU*fA*fA*fA*fA*fA*mC*fA*fG*fC*fA* | | |
| | mU*fU*mU*fG*fC* fAfAmU* dA*dC*dA mUfUfC | | |
| | *fA*fA*fA*mU*mC*mC | | |
| V117.40 | C6 Aminolinker - | 98 | 14 |
| | fG*fU*mC*fC*fA*mU*fU*fA*fA*fA*fA*fA*mC*fA*fG*fC*fA*mU*fU*mU* | | |
| | fG*fC*fAfAmU* dA*dC*dA mUfUfC *fA*fA | | |
| V117.41 | C6 Aminolinker - | 99 | 14 |
| | dG*fU*mC*fC*fA*mU*fU*fA*fA*fA*A*A*mC*fA*dG*fC*fA*mU*fU*mU* | | |
| | dG*fC*fAfAmU* dA*dC*dA mUfUfC *fA*fA | | |
| V117.42 | C6 Aminolinker - | 100 | 14 |
| | fU*mC*fC*fA*mU*fU*fA*fA*A*fA*fA*mC*fA*dG*fC*fA*mU*fU*mU*dG* | | |
| | fC*fAfAmU* dA*dC*dA mUfUfC *fA*fA | | |
| V117.43 | C6 Aminolinker - | | 14 |
| | mC*fC*fA*mU*fU*A*A*fA*fA*fA*mC*fA*dG*fC*fA*mU*fU*mU*dG*fC*f | 101 | |
| | AfAmU* dA*dC*dA mUfUfC *fA*fA | | |
| V117.44 | C6 Aminolinker- | 102 | 14 |
| | mU*mU*mU*dG*fU*mC*fC*fA*mU*fU*fA*fA*fA*fA*fA*mC*A*dG*fC*fA | | |
| | *mU*fU*mU*dG*fC* fAfAmU* dA*dC*dA mUfUfC *fA*fA*fA*mU | | |

As shown in FIGS. 14A and 14C, a 45 nt long version (v120.2), comprising 2′-F, 2′-OMe and 2′-H modifications outside of the CBT, exhibited good RNA editing efficacy (50.9% and 49.9%). Notably, truncation from the 5′ terminus led to a gradual decrease in editing (v117.26 (15%) and v117.28 (18.7%)). Notably, extension of the 3′ terminus led to an overall increase in editing efficacy (v117.24 (37.2%) and v117.25 (38.2%)) (FIG. 14A). These data show that truncation of the 5′ terminus of the oligonucleotide may have a significant impact on the editing efficacy of the ASO. Furthermore, the data suggest that a higher 2′-F content (>50%) in 40 nt long ASOs correlates with higher editing values (v117.27, contains 52.5% 2′-F). Notably, all embodiments do not contain uniform modification blocks larger than 6 nt.

The inventors went on to test shorter versions of CRB1 C948Y targeting constructs in order to assess whether there is a correlation between ASO length and overall 2′ modification pattern and editing efficacy. To do so, a set of shorter CRB1 C948Y targeting constructs were generated (v117.39 to v117.44) (Table 13) and tested for their in vitro editing efficacy. As shown in FIG. 14C, a DNA content of about 20% (5 DNA nucleosides outside of the CBT) in a 40 nt long ASO (i.e., v117.26) led to a decrease in editing. However, further shortening to a length of 38 nt (v117.44), the inventors discovered a “38 nt sweet spot”. For example, for v117.44 (38 nt long), the inventors discovered that an increase in editing yields to about 33% compared to the similar 40 nt embodiment v117.27 (27.2% editing yield). As also seen in Example 12, these shorter embodiments (≤50 nt) also tolerate very low amounts of 2′-OMe (21.2% in v117.40 and v117.41). Additionally, the inventors found certain short (e.g., ≤50 nt) embodiments where a low level (e.g., 3 nt outside the CBT) of DNA can improve editing yields compared to a pure 2′-F and 2′-OMe version, e.g., v117.27 (15% DNA/three DNA nucleosides outside of the CBT) versus v117.39 (7.5% DNA/no DNA nucleosides outside of the CBT) and v117.40 (9.1% DNA/no DNA nucleosides outside of the CBT) versus v117.41 (18.2% DNA/three DNA nucleosides outside of the CBT).

Overall, while these data confirm that ASOs comprising, e.g., no more than 6 consecutive nucleotides with the same modification and a mixture of 2′-F- and 2′-OMe-modifications have good editing efficacy, the data further suggest that a certain level of DNA may be better than a pure 2′-F- and 2′-OMe-modied version.

### Example 14. Modification Preference of Short (<45 nt) STAT1 Y701 Targeting ASOs after Shortening

To determine whether the replacement of DNA by 2′-OMe modification(s) influences RNA editing in a position-dependent manner, various ASOs were generated and tested for their STAT1 Y701 RNA editing efficacy. The different construct sequences and their modifications are listed in Table 14. Results are shown in FIG. 15. “no ASO” served as negative control.

TABLE 14
STAT1 Y701 targeting construct sequences and modifications used in
Example 14. mN = 2′-O-methyl (2′-OMe), fN = 2′-fluoro (2′-F), N = 2′-OH (ribose; RNA),
dN = 2′-H DNA), * = phosphorothioate (PS) linkage.

| Construct | | Seq. | FIG. |
|---|---|---|---|
| Name | Construct Sequence (5′ to 3′ direction) | ID | No. |
| V117.42 | mC*mA*mG*fA*fC*fA*mC*fA*dG*fA*fA*fA*fU*mC*fA*fA*fC*mU*fC*fA* | 83 | 15 |
| | dG* mU*fC*mU*fU*dGfAmU* dA*dC*dA mUfC | | |
| | fC*fA*dG*mU*fU*mC*fC | | |
| V117.43 | mC*mA*mG*fA*fC*fA*mC*fA*mG*fA*fA*fA*fU*mC*fA*fA*fC*mU*fC*fA | 103 | 15 |
| | *mG*mU*fC*mU*fU*mGfAmU* dA*dC*dA mUfC | | |
| | fC*fA*mG*mU*fU*mC*fC | | |
| V117.44 | mC*mA*mG*fA*fC*fA*mC*fA*fG*A*fA*A*fU*mC*fA*fA*fC*mU*fC*fA* | 84 | 15 |
| | fG*mU*fC*mU*fU*fGfAmU* dA*dC*dA mUfC fC*fA*fG*mU*fU*mC*fC | | |
| V117.45 | mC*mA*mG*fA*fC*fA*mC*fA*dG*fA*fA*fA*fU*mC*fA*fA*fC*mU*fC*fA* | 104 | 15 |
| | mG*mU*fC*mU*fU*mGfAmU* dA*dC*dA mUfC | | |
| | fC*fA*dG*mU*fU*mC*fC | | |
| V117.46 | mC*mA*mG*fA*fC*fA*mC*fA*mG*fA*fA*fA*fU*mC*fA*fA*fC*mU*fC*fA | 105 | 15 |
| | *dG*mU*fC*mU*fU*dGfAmU* dA*dC*dA mUfC | | |
| | fC*fA*mG*mU*fU*mC*fC | | |
| V117.47 | mC*mA*mG*fA*fC*fA*mC*fA*mG*fA*fA*fA*fU*mC*fA*A*fC*mU*fC*fA | 106 | 15 |
| | *mG*mU*fC*mU*fU*dGfAmU* dA*dC*dA mUfC | | |
| | fC*fA*dG*mU*fU*mC*fC | | |
| V117.48 | mC*mA*mG*fA*fC*fA*mC*fA*dG*fA*fA*fA*fU*mC*fA*fA*fC*mU*fC*fA* | 107 | 15 |
| | dG* mU*fC*mU*fU*mGfAmU* dA*dC*dA mUfC | | |
| | fC*fA*mG*mU*fU*mC*fC | | |

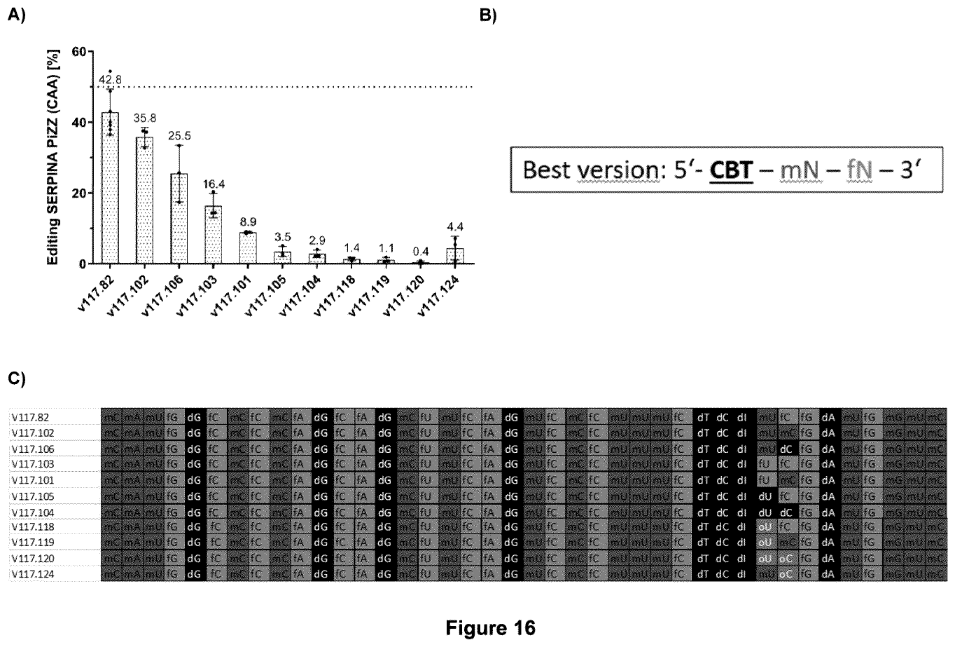

The data demonstrate that the replacement of DNA by 2′-OMe modification(s) influences editing in a position-dependent manner. As shown in FIG. 16A, 2′-OMe modifications are preferred over DNA close to the CBT (from N₋₉ to N₊₉, v117.48 versus v117.47). It was also shown that all four DNA nucleotides outside of the CBT (see, v117.42) could be replaced by 2′-F-modifications (in v117.44) keeping the largest block at s 6 nt to improve editing yields to a level of 64%, similar to that of construct v117.48 (65.2%) (FIG. 16A), showing that 2′-F modifications are generally well tolerated and even better tolerated than for the 59 nt ASO (see, FIG. 3, 17.3% editing efficacy for v117.41). However, 2′-F-modified nucleosides outside the CBT could also be replaced by 2′-OMe and/or DNA nucleosides (e.g., 2 DNA nt without loss of editing efficiency, v117.48) in the short ASOs (≤50 nt) to some extent to, e.g., reduce the overall 2′-F/PS content, which can cause toxicity in certain sequence contexts. Nevertheless, the effect of replacing 2′-F-modifications by other 2′-modifications on editing efficiency can be very position-specific. Again, avoidance of blocks of uniform 2′-modification (≤6 nt) seems to be preferred. Additionally, the data also indicates that higher levels of 2′-OMe (up to 40% in v117.43) are generally well tolerated and may only have a minor impact on editing yields when correctly placed.

### Example 15. Identification of Hotspot Site 3′ to the Central Base Triplet (CBT) of SERPINA1 E342K Targeting ASOs

As previously described by the inventors of the present application selection of the nucleotide at the first position 3′ of the traditional CBT (at the +2 position in a structure of [Am]-N₋₁-N₀-N₊₁-N₊₂-[Bn], where N₀ is the editing site) can significantly impact the editing rate of the target. To further examine the effect of nucleotide modification 3′ to the CBT, different SERPINA1 targeting constructs were generated that carried nucleotide modifications at the first two nucleotides directly 3′ to the CBT (i.e., positions +2 and +3) (5′-^{d}N₋₁^{e}N₀^{f} N₊₁^{g}N₊₂^{h}N₊₃ⁱ-′3). Generally, the different SERPINA1 targeting constructs carry the same base sequence, with differences only in positions +2 and +3 (Table 15 and FIG. 16B). The results are shown in FIG. 16. The various SERPINA1 targeting ASOs and their modifications are listed in Table 15.

As shown in FIG. 16A, construct v117.82, which contains a 2′-O-methyl modification at position +2 (N₊₂) and a 2′-fluoro modification at position +3 (N₊₃), showed, with a value of 42.8%, the highest level of editing efficacy. On the other hand, replacing the 2′-O-methyl modification at the +2 position with either a 2′-F, 2′-H or 2′-MOE modification led to a gradual decrease in SERPINA1 editing efficacy. Similarly, ASO constructs that carried either a 2′-OMe, 2′-H or 2′-MOE modification at the +3 position instead of the 2′-fluoro modification showed a decrease in SERPINA1 editing efficacy, dropping down to editing levels of as low as 0.4% (see, v117.101, v117.102, v117.104, v117.106, v117.119, v117.120, and v117.124). Interestingly, having a 2′-MOE modification at either the +2 position (v117.118 and v117.119) or at the +2 and +3 positions (v117.120) lead to the greatest decrease in editing efficacy (FIG. 16A). These data suggest that that a 2′-O-methyl modification at +2, and a 2′-F modification at the +3 position are needed to provide optimal editing efficacy. Particularly important is the +2 position, followed by the +3 position.

Overall, these data suggest that the types of 2′-modification at the first two nucleosides directly 3′ to the CBT (5′-^{d} N₋₁^{e}N₀^{f}N₊₁^{g}-′3), i.e., N₊₂ and N₊₃, play an important role in determining the editing efficacy of the ASO. Interestingly, the inventors thus discovered a modification-sensitive “hot spot” region directly 3′ to the CBT at positions +2 and +3. Hence, the inventors submit that the CBT together with the first two nucleosides directly 3′ to the CBT form an “extended CBT” (5′-^{d}N₋₁^{e}N₀^{f} N₊₁^{g} N₊₂^{h}N₊₃ⁱ-′3), wherein the best arrangement and/or modification to obtain efficient target editing is
5′-CBT-mN-fN-3′,
   wherein CBT refers to the central base triplet, mN is a 2-O-methyl-modification, and fN is 2′-fluoro-modification. The organisational structure of this extended modification-sensitive “hot spot” is also shown in FIG. 16B.

Interestingly, this extended CBT is also sensitive to the internucleoside linkage modification, accepting PS modification very well at linkages d and e, well at f and/or g, but does not accept the PS well at position h and i. Accordingly, in one embodiment, d and e are PS linkage modifications. In one embodiment, f is a PS linkage. In one embodiment, g is a PS linkage. In some cases, h and i are not PS linkages.

TABLE 15
SERPINA1 E342K targeting construct sequences and modifications used
for assessment of Hotspot site 3′ of the CBT in Example 15. mN = 2′-O-methyl (2′-
OMe), fN = 2′-fluoro (2′-F), oN = 2′-MOE, N = 2′-OH (ribose; RNA), dN = 2′-H (deoxyribose;
DNA), * = phosphorothioate (PS) linkage.

| Construct | | Seq. | FIG. |
|---|---|---|---|
| Name | Construct Sequence (5′ to 3′ direction) | ID | No. |
| V117.82 | mC*mA*mU*fG*dG*fC*mC*fC*mC*fA*dG*fC*fA*dG*mC*fU*mU* | 63 | 16 |
| | fC*fA*dG*mU*fC*mC*fC*mU*mUmUfC* dT*dC*dI mU fC fG* dA* | | |
| | mU* fG* mG* mU* mC | | |
| V117.101 | mC*mA*mU*fG*dG*fC*mC*fC*mC*fA*dG*fC*fA*dG*mC*fU*mU* | 108 | 16 |
| | fC*fA*dG*mU*fC*mC*fC*mU*mUmUfC* dT*dC*dI fU mC fG* dA* | | |
| | mU* fG* mG* mU* mC | | |
| V117.102 | mC*mA*mU*fG*dG*fC*mC*fC*mC*fA*dG*fC*fA*dG*mC*fU*mU* | 109 | 16 |
| | fC*fA*dG*mU*fC*mC*fC*mU*mUmUfC* dT*dC*dI mU mC fG* | | |
| | dA* mU* fG* mG*mU*mC | | |
| V117.103 | mC*mA*mU*fG*dG*fC*mC*fC*mC*fA*dG*fC*fA*dG*mC*fU*mU* | 110 | 16 |
| | fC*fA*dG*mU*fC*mC*fC*mU*mUmUfC* dT*dC*dI fU fC fG* dA* | | |
| | mU* fG* mG* mU* mC | | |
| V117.104 | mC*mA*mU*fG*dG*fC*mC*fC*mC*fA*dG*fC*fA*dG*mC*fU*mU* | 111 | 16 |
| | fC*fA*dG*mU*fC*mC*fC*mU*mUmUfC* dT*dC*dI dUdC fG* dA* | | |
| | mU* fG* mG* mU* mC | | |
| V117.105 | mC*mA*mU*fG*dG*fC*mC*fC*mC*fA*dG*fC*fA*dG*mC*fU*mU* | 112 | 16 |
| | fC*fA*dG*mU*fC*mC*fC*mU*mUmUfC* dT*dC*dI dU fC fG* | | |
| | dA*mU* fG* mG* mU*mC | | |
| V117.106 | mC*mA*mU*fG*dG*fC*mC*fC*mC*fA*dG*fC*fA*dG*mC*fU*mU* | 113 | 16 |
| | fC*fA*dG*mU*fC*mC*fC*mU*mUmUfC* dT*dC*dI mU dC fG* | | |
| | dA* mU* fG* mG*mU*mC | | |
| V117.118 | mC*mA*mU*fG*dG* fC*mC*fC*mC*fA*dG*fC*fA* dG*mC* fU* | 114 | 16 |
| | mU* fC* fA* dG*mU*fC*mC*fC*mU*mUmUfC* | | |
| | dT*dC*dIoUfCfG*dA*mU*fG*mG*mU*mC | | |
| V117.119 | mC*mA*mU*fG*dG* fC*mC* fC* mC* fA* dG* fC* fA* dG*mC* | 115 | 16 |
| | fU* mU* fC* fA* dG*mU*fC*mC*fC*mU*mUmUfC* dT*dC*dIoU | | |
| | mCfG* dA* mU*fG*mG*mU*mC | | |
| V117.120 | mC*mA*mU*fG*dG* fC*mC*fC*mC*fA*dG*fC* fA* dG* mC* | 116 | 16 |
| | fU*mU* fC* fA* dG* mU*fC*mC*fC*mU*mUmUfC* dT*dC*dI oU | | |
| | oC fG* dA* mU* fG* mG* mU*mC | | |
| V117.124 | mC*mA*mU*fG*dG* fC*mC*fC*mC*fA*dG*fC*fA* | | |
| | dG*mC*fU*mU* fC* fA* dG*mU*fC*mC*fC*mU*mUmUfC* | 117 | 16 |
| | dT*dC*dI mU oC fG*dA* mU* fG* mG* mU*mC | | |

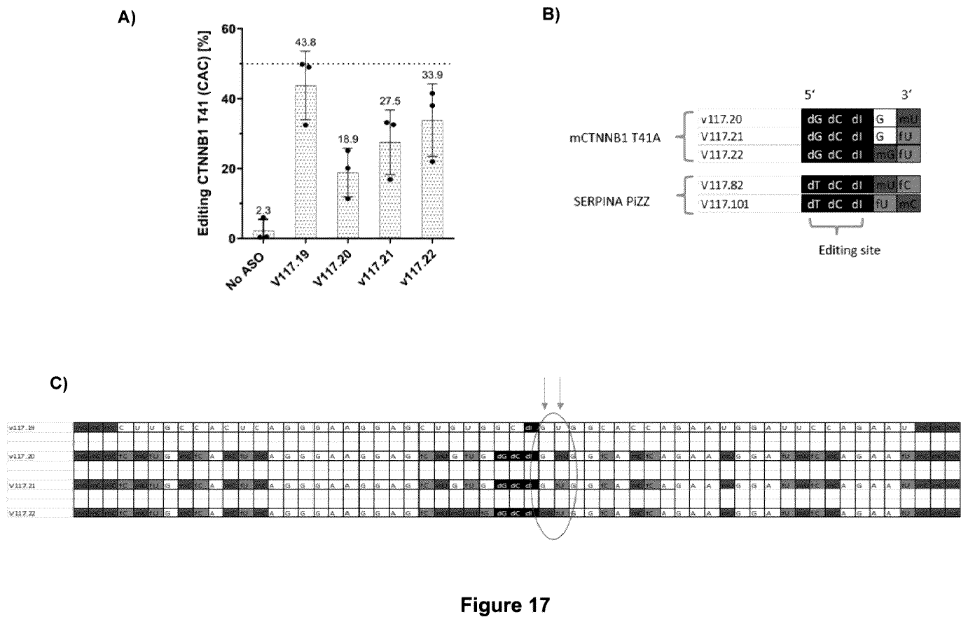

To further investigate the extended hot spot region, CTNNB1 T41A targeting ASOs carrying 2′-O-methyl or 2′-F-modifications at the +2 or +3 positions were assessed for their in vitro editing efficacy. The sequence modifications of the different ASOs tested are shown in Table 16 and the results are shown in FIG. 17. “no ASO” served as negative control.

TABLE 16
murine CTNNB1 T41A targeting construct sequences and modifications
used in Example 15. mN = 2′-O-methyl (2′-OMe), fN = 2′-fluoro (2′-F), oN = 2′-MOE, N =
2′-OH (ribose; RNA), dN = 2′-H (deoxyribose; DNA), * = phosphorothioate (PS) linkage.

| Construct | | Seq. | FIG. |
|---|---|---|---|
| Name | Construct Sequence (5′ to 3′ direction) | ID | No. |
| V117.19 | mG*mC*mC*C*U*U*G*C*C*A*C*U*C*A*G*G*G*A*A*G*G*A*G* | 118 | 17 |
| | C*U* GUG GCdI GUG* G* C*A* C*C* A*G* A*A* U* G* G* A* U* | | |
| | U* C* C* A* G* A* A* U* mC* mC* mA | | |
| V117.20 | mG*mC*mC*fC*mU*fU*G*mC*fC*A*mC*fU*mC*A*G*G*G*A*A* | 119 | 17 |
| | G*G*A*G*fC*mU* GfUG* dG*dC*dI GmUG* G*fC* A* mC* fC* A* | | |
| | G* A* A* | | |
| V117.21 | mG*mC*mC*fC*mU*fU*G*mC*fC*A*mC*fU*mC*A*G*G*G*A*A* | 120 | 17 |
| | G*G*A*G*fC*mU* GfUG* dG*dC*dI GfUG* G* fC* A* mC* fC* | | |
| | A*G*A*A* mU* G* G* A* fU* mU* fC* mC* A* G* A* A* fU* mC* | | |
| | mC*mA | | |
| V117.22 | mG*mC*mC*fC*mU*fU*G*mC*fC*A*mC*fU*mC*A*G*G*G*A*A* | 87 | 17 |
| | G*G*A*G*fC*mU* mGmUfG* dG*dC*dI mGfUG* G*fC* A*mC* | | |
| | fC* A*G*A*A* mU* G* G* A* fU* mU* fC* mC* A* G* A* A* fU* | | |
| | mC*mC*mA | | |

As shown in FIG. 17A, construct v117.22, which carries a 2′-O-methyl modification at position +2 and a 2′-F-modification at position +3 showed an editing efficacy of 33.9%, which was higher than the editing efficacy of constructs having a 2′-OH at position +2 and either a 2′-O-methyl at position +3 (v117.20 (18.9%)) or a 2′-F-modification at position +3 (v117.21 (27.5%)).

Hence, these data demonstrate and confirm that the optimal mixture of combinations and their arrangement for good maintenance of RNA editing is as defined by the extended hotspot having a structure of 5′-CBT-mN-fN-3′.

### Example 16. Replacement of 2′-OMe-Modified Endblocks with 2′-MOE in SERPINA1 E342K Targeting ASOs

It is known that 2′-MOE residues are heavily used for splice switching oligonucleotides and typically have very low toxicity. Due to their bulkiness and location in the minor groove, they are not well accepted in larger quantities and are not at all accepted in certain positions of ADAR recruiting ASOs. To determine the effect of 2′-MOE modifications on the stability, toxicity and editing efficacy different SEPINA1 targeting constructs were generated and tested for their editing efficacy and lysosomal stability.

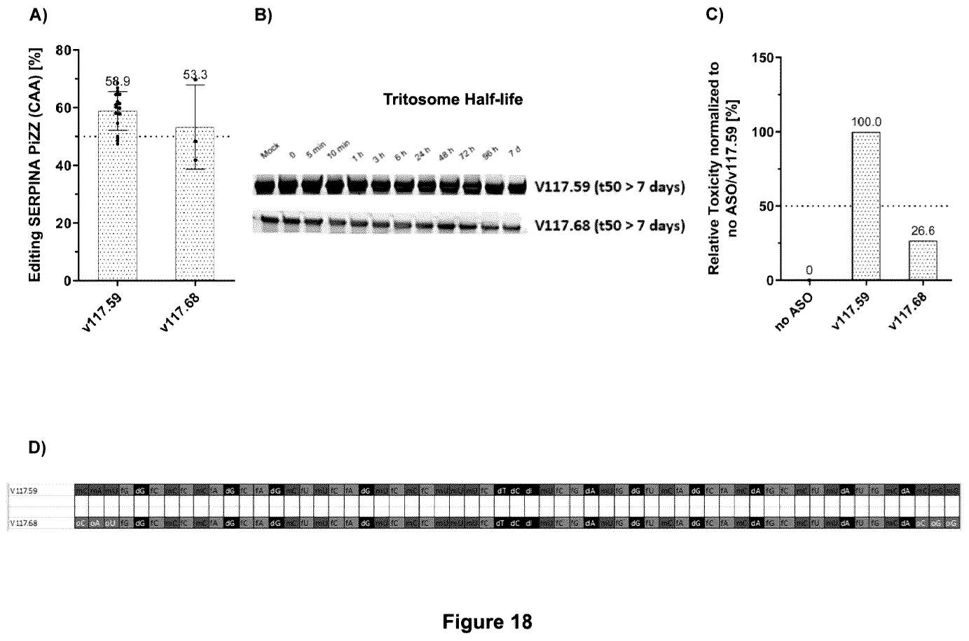

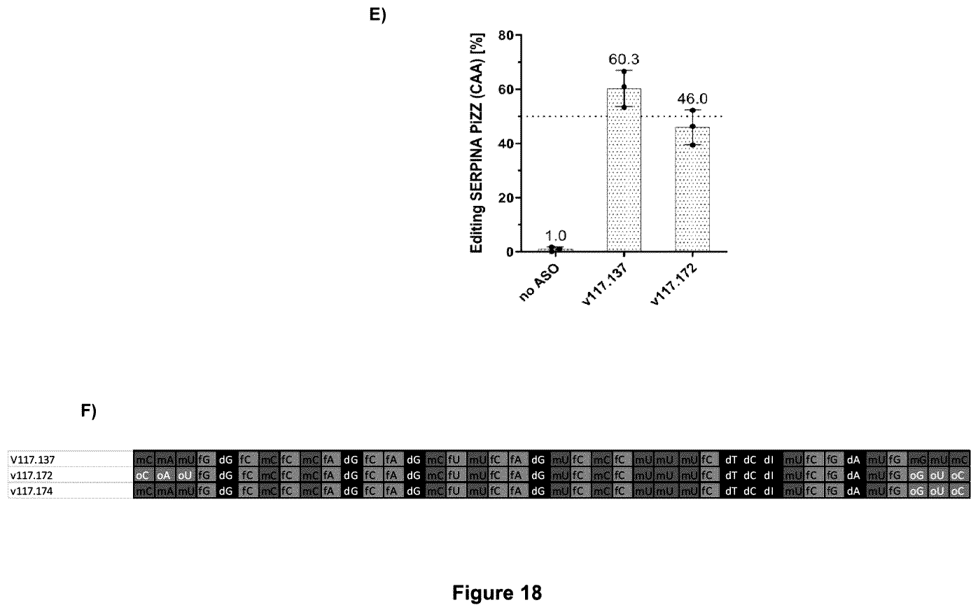

The different ASOs and their sequence modifications are listed in Table 17. Results are shown in FIG. 18. “no ASO” served as negative control. Toxicities were quantified with the CellTox™ Green Cytotoxicity Assay (Promega) according to manufacturer's protocol, then normalized to the negative (“no ASO”) and positive control (v117.59).

Initial testing involved the generation of a 59 nt long ASO comprising a total of six 2′-MOE nucleotides, wherein three 2′-MOE nucleotides were placed successively at the end of each terminus (v117.68). This ASO variant was based on a 59 nt lead ASO with an 85% PS-modified backbone (v117.59). Notably, introducing 2′-MOE modifications at the termini of the lead ASO did not affect overall editing yield (FIG. 18A). Similarly, the lysosomal half-life of the v117.68 variant was not impacted when compared to the control, with both constructs showing a t₅₀>7 days (FIG. 18B). However, as shown in FIG. 18C, cellular toxicity of the test construct was surprisingly reduced to only 26.6% when compared to the v117.59 control.

Similarly, 2′-MOE modifications were introduced into a 40 nt long ASO in the same manner as v117.68. This ASO variant was based on a 40 nt lead ASO, also containing an 85% PS-modified backbone (v117.137). Results are shown in FIG. 18E. Editing yields were somewhat lower in the construct containing 2′-MOE termini versus the construct without. The lowered editing yields in v117.172 may arise from the closer placement of the 2′-MOE modifications to the CBT, a well-known contact site of the ADAR enzyme. Without being bound to any theory, the inventors suggest that 2′-MOE modifications should be placed further away from known enzyme contact sites to prevent an interference of the protein binding through the bulky 2′-MOE modification side chain.

TABLE 17
SERPINA1 E342K targeting construct sequences and modifications
used in Example 16. mN = 2′-O-methyl (2′-OMe), fN = 2′-fluoro (2′-F), oN = 2′-MOE, N =
2′-OH (ribose; RNA), dN = 2′-H (deoxyribose; DNA), * = phosphorothioate (PS) linkage;
oN = 2′-MOE.

| Construct | | Seq. | FIG. |
|---|---|---|---|
| Name | Construct Sequence (5′ to 3′ direction) | ID | No. |
| V117.59 + | C6-Aminolinker - | 155 | 18 |
| Amino | mC*mA*mU*fG*dG*fC*mC*fC*mC*fA*dG*fC*fA*dG*mC*fU*mU*fC*fA | | |
| | *dG*mU*fC*mC*fC*mU*mUmUfC* dT*dC*dI mU fC fG* dA* mU* fG* | | |
| | dG* fU* mC* fA* dG* fC* fA* mC*dA*fG*fC* mC*fU*mU*dA*fU* fG* | | |
| | mC* dA* mC* mG* mG | | |
| V117.68 | C6-Aminolinker - | 121 | 18 |
| | oC*oA*oU*fG*dG*fC* mC* fC* mC* fA* dG*fC* fA* dG*mC* fU* | | |
| | mU*fC*fA*dG*mU*fC*mC*fC*mU*mUmUfC*dT*dC*dI mU fC fG* | | |
| | dA*mU* fG* dG*fU*mC*fA* dG*fC* fA* mC* dA* fG* fC* mC* fU* mU* | | |
| | dA*fU*fG*mC*dA*oC*oG*oG | | |
| V117.137 | C6-Aminolinker - | 122 | 18 |
| | mC*mA*mU*fG*dG*fC*mC*fC*mC*fA*dG*fC*fA*dG*mC*fU*mU*fC*fA | | |
| | *dG*mU*fC*mC*fC*mU*mUmUfC* dT*dC*dImU fC fG* dA* mU* fG* | | |
| | mG* mU* mC | | |
| V117.172 | C6-Aminolinker - | 123 | 18 |
| | oC*oA*oU*fG*dG*fC*mC*fC*mC*fA*dG*fC*fA*dG*mC*fU*mU*fC*fA* | | |
| | dG*mU*fC*mC*fC*mU*mUmUfC* dT*dC*dI mU fC fG* dA* mU* fG* | | |
| | oG*oU*oC | | |
| V117.74 | C6-Aminolinker - | 124 | 18 |
| | mC*mA*mU*fG*dG*fCmCfCmCfA*dG*fCfA*dG*mCfUmUfCfA*dG*m | | |
| | UfCmCfCmUmUmUfC* dT*dC*dI*mUfCfG*dA*mUfG* dG*fUmC fA* | | |
| | dG*fC fA mC*dA*fGfCmCfUmU*dA*fUfGmC*dA*mC*mG*mG | | |

These data demonstrate that the presence of 2′-MOE endblocks at the 5′ and 3′ termini of the ASO impact overall ASO activity. For instance, the findings suggest that 5′- and 3′-terminal modification of the ASO with 2′MOE may help in enhancing lysosomal stabilization. Moreover, the data demonstrate that 5′- and 3′-terminal 2′-MOE endblocks can be incorporated without impacting editing efficiency while clearly reducing cellular toxicity for long (>40 nt) embodiments. A small reduction in editing efficacy is seen for shorter embodiments containing terminal 2′-MOE modifications versus embodiments containing less bulky 2′-modifications, e.g., 2′-OMe.

### Example 17. Reduction of PS Linkage Backbone Modifications in SERPINA1 E342K Targeting ASOs

Phosphorothioate (PS) linkages can add beneficial properties to ASOs by improving albumin binding, cellular uptake, endosomal escape, and protein binding. Moreover, PS linkages have been reported to shift ASOs from the cytosol to the nucleoplasm (Crooke et al., 2020). On the downside, PS linkages tend to make ASOs sticky, which can lead to protein and/or ASO aggregation and toxicities. For example, an increase in PS-related toxicity has been observed in ASOs rich of 2′-F modifications. As a result, there is an increasing interest in learning how to modify ASOs with PS linkages to ultimately reduce the amount of PS linkages and/or 2′-F content within each ASO.

To test four different ASO constructs with reduced PS content and to determine the impact of reduced PS linkage modifications on editing efficacy, two different model systems [plasmid-based (A) and genomically integrated approach (B)] were used.

Plasmid-transfected approach (A): 2.5×10⁴ HeLa cells were seeded in a 24-well plate. After 24 h, cells were forward transfected with a plasmid containing the human SERPINA1 E342K (PiZZ) mutated cDNA. Forward transfection was performed by diluting 300 ng plasmid and 0.9 μl FuGENE® 6 (Promega) each in 50 μl Opti-MEM and incubating for 5 min, then combining both mixtures and incubating for an additional 20 min. The medium was changed, and the transfection mix evenly distributed into one well. 24 h after plasmid transfection, cells were forward transfected with 5 pmol construct/well and 1.5 μl/well Lipofectamine RNAiMAX Reagent (ThermoFisher Scientific). Forward transfection of the ASO constructs occurred by mixing the construct and lipofectamine reagent in 50 μL OptiMEM each. Both solutions were combined after 5 min incubation and incubated for an additional 20 min. Medium was changed before the transfection mix was distributed evenly into one well. After 24 h, medium was changed again. 48 h after transfection, cells were harvested for RNA isolation and prepared for Sanger sequencing.

Genomically integrated approach (B): 1×10⁵ HeLa cells containing the human SERPINA1 E342K mutated cDNA gene stably integrated into their genome via the piggyBac transposase system were seeded in a 24-well plate. After 24 h cells were forward transfected (procedure described above) with 25 pmol construct/well and 1.5 μl/well Lipofectamine RNAiMAX Reagent (ThermoFisher Scientific) according to the Lipofectamine RNAiMAX protocol. After 24 h, cells were harvested for RNA isolation and Sanger sequencing.

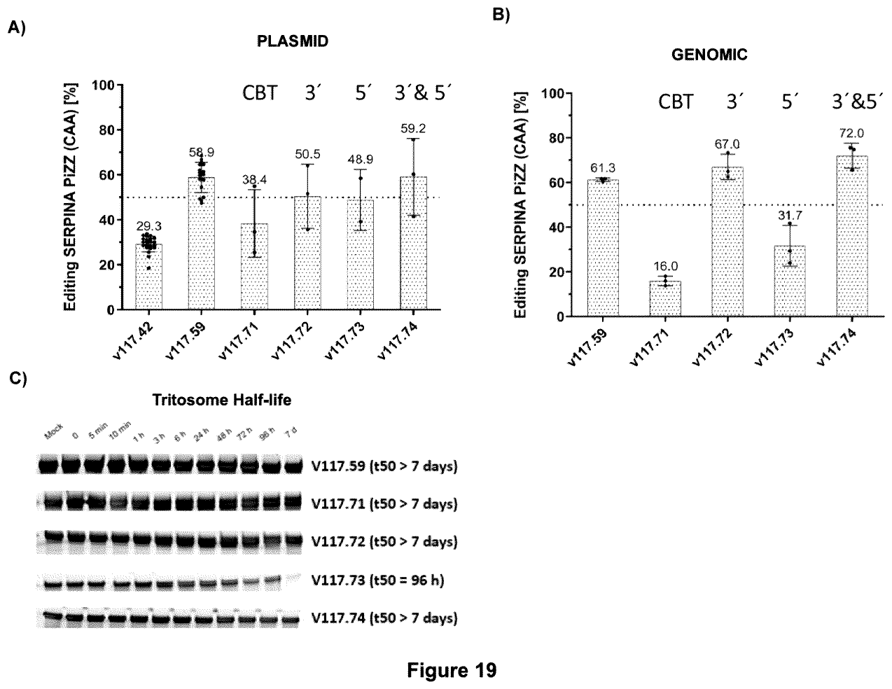

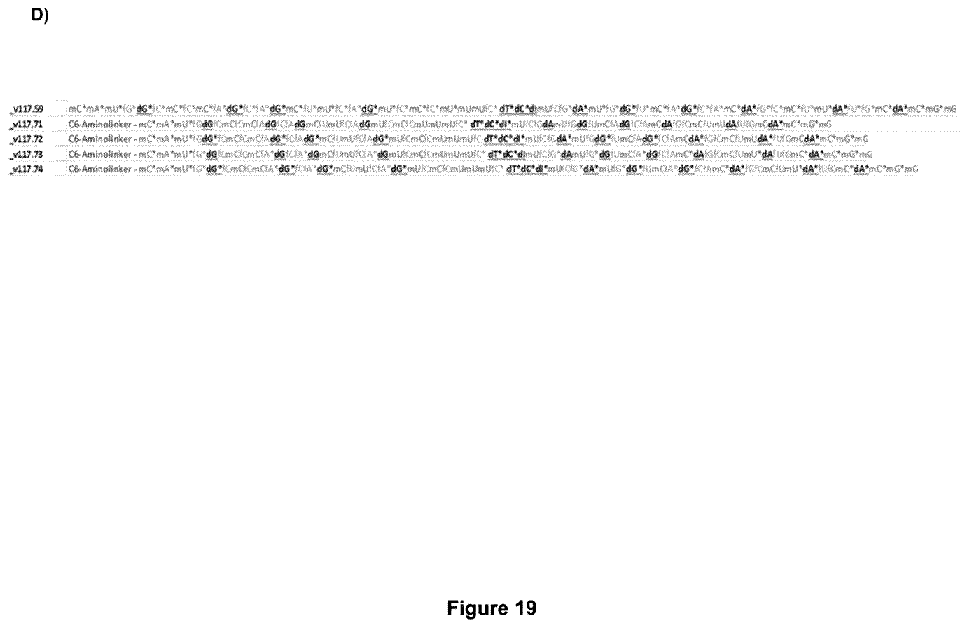

The different test constructs were compared to a PS-rich lead construct (v117.59, 59 nt long) with about 90% PS content. In the most extreme construct, PS linkages were included only at the 5′ and 3′ termini (2×3 PS linkages each) and around the CBT (4 PS) providing an overall PS content of only about 17% (v117.71). While this PS-poor ASO still had notable lysosomal stability (FIG. 19C, t₅₀>7 days) and gave notable editing, this was however, strongly reduced compared to the PS-rich lead (v117.59) (FIG. 19A and FIG. 19B).

To further assess the impact of varying levels of PS content on ASO activity and stability, additional PS linkages were introduced across the ASO either 5′ or 3′ of each DNA base (see, v117.72 and v117.73) (Table 18 and FIG. 19D). This resulted in an overall PS content of about 30.5% (v117.72) and 32.2% (v117.73). As shown in FIGS. 19A and B, independent of the approach used, increasing the overall PS content mediated a clear improvement in the editing yield when compared to v117.71.

Furthermore, reducing the overall PS content within each ASO did not drastically reduce tritosomal stability (t₅₀>7 days). Notably, it was found that PS linkages 3′ to DNA are more important than 5′ to DNA in terms of tritosomal stability (FIG. 19C, v117.73, t₅₀=96 h). Introducing PS linkages 5′ and 3′ to each DNA base generated an ASO with only 49.2% PS content (v117.74), which however, was similar to the PS-rich lead construct v117.59 in terms of tritosomal stability (t₅₀>7 days) and editing efficiency (FIG. 19).

TABLE 18
SERPINA1 E342K targeting construct sequences and modifications used
in Example 17. mN = 2′-O-methyl (2′-OMe), fN = 2′-fluoro (2′-F), N = 2′-OH (ribose; RNA),
dN = 2′-H (deoxyribose; DNA), * = phosphorothioate (PS) linkage.

| Construct | | Seq. | FIG. |
|---|---|---|---|
| Name | Construct Sequence (5′ to 3′ direction) | ID | No. |
| V117.42 | C6-Aminolinker - | 3 | 19 |
| | mC*mA*mU*dG*dG*fC*mC*fC*mC*dA*dG*fC*dA*dG*mC*fU*mU*fC* | | |
| | dA*dG*mU*fC*mC*fC*mU*mUmUfC* dT*dC*dImUfC dG*dA*mU* | | |
| | dG*dG*fU* mC* dA*dG*fC* dA*mC*dA*dG*fC* mC* fU* mU* | | |
| | dA*fU*dG*mC*dA*mC*mG*mG | | |
| V117.59 | mC*mA*mU*fG*dG*fC*mC*fC*mC*fA*dG*fC*fA*dG*mC*fU*mU*fC*fA* | 20 | 19 |
| | dG*mU*fC*mC*fC*mU*mUmUfC* dT*dC*dImUfCfG*dA*mU*fG*dG*fU* | | |
| | mC* fA* dG* fC* fA*mC* dA* fG* fC* mC* fU* mU* dA* fU* fG* mC* | | |
| | dA* mC* mG* mG | | |
| V117.71 | C6-Aminolinker - | 125 | 19 |
| | mC*mA*mU*fGdGfCmCfCmCfAdGfCfAdGmCfUmUfCfAdGmUfCmCfC | | |
| | mUmUmUfC* dT*dC*dI* mU fC fG dA mU fG dG fU mC fA dG fC fA | | |
| | mC dAfG fC mC fU mUdAfUfGmCdA*mC*mG*mG | | |
| V117.72 | C6-Aminolinker - | 126 | \|19 |
| | mC*mA*mU*fGdG*fCmCfCmCfAdG*fCfAdG*mCfUmUfCfAdG*mUfCm | | |
| | CfCmUmUmUfC dT*dC*dI*mU fC fG dA* mU fG dG* fU mC fA dG* fC | | |
| | fA mC dA*fGfCmCfUmUdA*fUfGmCdA*mC*mG*mG | | |
| V117.73 | C6-Aminolinker - | 127 | \|19 |
| | mC*mA*mU*fG*dGfCmCfCmCfA*dGfCfA*dGmCfUmUfCfA*dGmUfCm | | |
| | CfCmUmUmUfC* dT*dC*dImUfCfG* dA mU fG* dG fU mC fA* dG fC | | |
| | fA mC* dA fG fC mC fUmU*dAfUfGmC*dA*mC*mG*mG | | |
| V117.74 | C6-Aminolinker - | 124 | \|19 |
| | mC*mA*mU*fG*dG*fCmCfCmCfA*dG*fCfA*dG*mCfUmUfCfA*dG*mUf | | |
| | CmCfCmUmUmUfC* dT*dC*dI* mU fC fG* dA* mU fG*dG* fU mC fA* | | |
| | dG*fC fA mC* dA*fGfCmCfUmU*dA*fUfGmC*dA*mC*mG*mG | | |

These findings demonstrate that the PS content in fully modified ASOs can be significantly reduced, without decreasing the editing efficacy. However, a decline in editing efficacy might be encountered. A linkage modification degree of at least 15% is required. For the long embodiments (e.g., ≥40 nt) the overall internucleoside content should be in the range of 15-90%, and editing and lysosomal stability are preserved at an internucleoside modification content as low as 30% (v117.72 and v117.73).

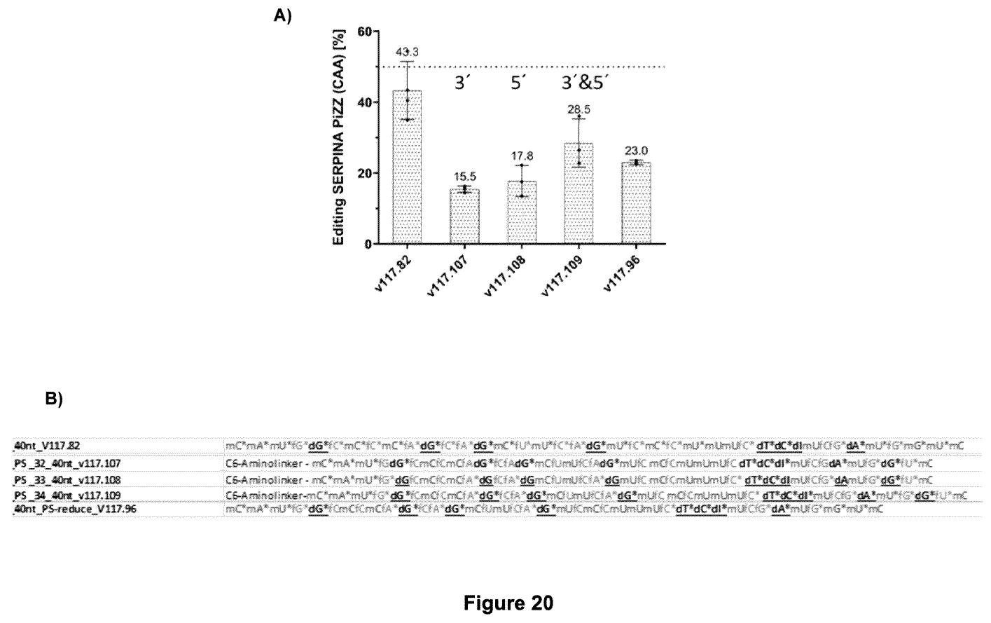

The inventors went on to test the effect of PS content on shorter ASOs (40 nt). The various tests constructs are listed in Table 19. Results are shown in FIG. 20.

TABLE 19
SERPINA1 E342K targeting construct sequences and modifications used
in Example 17. mN = 2′-O-methyl (2′-OMe), fN = 2′-fluoro (2′-F), N = 2′-OH (ribose; RNA),
dN = 2′-H (deoxyribose; DNA), * = phosphorothioate (PS) linkage.

| Construct | | Seq. | FIG. |
|---|---|---|---|
| Name | Construct Sequence (5′ to 3′ direction) | ID | No. |
| V117.82 | mC*mA*mU*fG*dG*fC*mC*fC*mC*fA*dG*fC*fA*dG*mC*fU*mU | 63 | 20 |
| | *fC*fA*dG*mU* fC*mC*fC*mU*mUmUfC* dT*dC*dI mU fC fG* | | |
| | dA* mU* fG* mG* mU* mC | | |
| V117.96 | mC*mA*mU*fG*dG*fCmCfCmCfA*dG*fCfA*dG*mCfUmUfCfA*dG* | 128 | 20 |
| | mUfC mC fC mU mUmUfC*dT*dC*dI*mUfC fG*dA*mU fG* mG* | | |
| | mU* mC | | |
| V117.107 | C6-Aminolinker - | 129 | 20 |
| | mC*mA*mU*fGdG*fCmCfCmCfAdG*fCfAdG*mCfUmUfCfAdG*mUf | | |
| | C mC fC mU mU mU fC dT*dC*dI*mUfCfGdA*mU fG dG*fU mC | | |
| V117.108 | C6-Aminolinker - | 130 | 20 |
| | mC*mA*mU*fG*dGfCmCfCmCfA*dGfCfA*dGmCfUmUfCfA*dGmUf | | |
| | C mC fC mU mU mU fC* dT*dC*dImUfCfG*dAmUfG*dGfUmC | | |
| V117.109 | C6-Aminolinker- | 131 | 20 |
| | mC*mA*mU*fG*dG*fCmCfCmCfA*dG*fCfA*dG*mCfUmUfCfA*dG* | | |
| | mUfC mC fC mU mUmUfC* dT*dC*dI*mUfCfG*dA*mU fG*dG*fU | | |
| | mC | | |

The 40 nt short lead ASO contained a PS linkage content of 85% (v117.82). Like for the longer ASOs, PS linkages were added only directly 3′, 5′, or 3′ and 5′ of each DNA nucleotide, thereby reducing the overall PS linkage content in the 40 nt short ASO to about 30% or 50%. Construct v117.96 is identical in sequence to v117.109, differing only in the last two nucleotides at the 3′ terminus (v117.96 contains two 2′-OMe modifications, which are absent in v117.109) and PS content, and serves as a control oligonucleotide for 50% PS content, since v117.82 also has the endblock at the 3′ terminus, which is absent from the other versions tested. Constructs v117.107, v117.108, and v117.109 expressed an overall PS content of about 30%, 30% and 47.5%, respectively. As shown in FIG. 20, v117.109 was not able to fully recover the editing yield of the PS-rich lead ASO, suggesting, that a reduction in PS content in the short ASO lead (40 nt) may be less well tolerated compared to the long ASO lead (59 nt).

Moreover, the inventors found that with the short ASOs (≤50 nt, e.g., 40 nt), a continuous stretch of linkage modifications (at least 10) were beneficial over a random spreading of linkage modifications when reducing the overall PS content. The different SERPINA1 E342K targeting constructs and their modifications are listed in Table 20. “no ASO” served as negative control.

TABLE 20
SERPINA1 E342K targeting construct sequences and modifications used
in Example 17. mN = 2′-O-methyl (2′-OMe), fN = 2′-fluoro (2′-F), N = 2′-OH (ribose; RNA),
dN = 2′-H (deoxyribose; DNA), * = phosphorothioate (PS) linkage

| Construct | | Seq. | FIG. |
|---|---|---|---|
| Name | Construct Sequence (5′ to 3′ direction) | ID | No. |
| V117.107 | C6-Aminolinker - | 129 | 21 |
| | mC*mA*mU*fGdG*fCmCfCmCfAdG*fCfAdG*mCfUmUfCfAdG*mU | | |
| | fC mC fCmUmUmUfC dT*dC*dI*mUfCfGdA*mUfGdG*fUmC | | |
| V117.82 | mC*mA*mU*fG*dG*fC*mC*fC*mC*fA*dG*fC*fA*dG*mC*fU*mU*fC | 63 | 21 |
| | * fA*dG*mU*fC*mC*fC*mU*mUmUfC* dT*dC*dImU fC fG* | | |
| | dA*mU*fG*mG*mU*mC | | |
| V117.132 | C6-Aminolinker - | 132 | 21 |
| | mC*mA*mU*fGdG*fC*mC*fC*mC*fA*dG*fC*fA*dG*mCfUmUfCfAdG* | | |
| | mU fCmCfCmUmUmUfC dT*dC*dI*mUfCfGdA*mUfGdG*fUmC | | |
| V117.133 | C6-Aminolinker - | 133 | 21 |
| | mC*mA*mU*fGdG*fC*mC*fC*mC*fA*dG*fC*fA*dG*mC*fU*mU*fC* | | |
| | fA*dG* mU fCmCfCmUmUmUfC | | |
| | dT*dC*dI*mUfCfGdA*mUfGdG*fUmC | | |

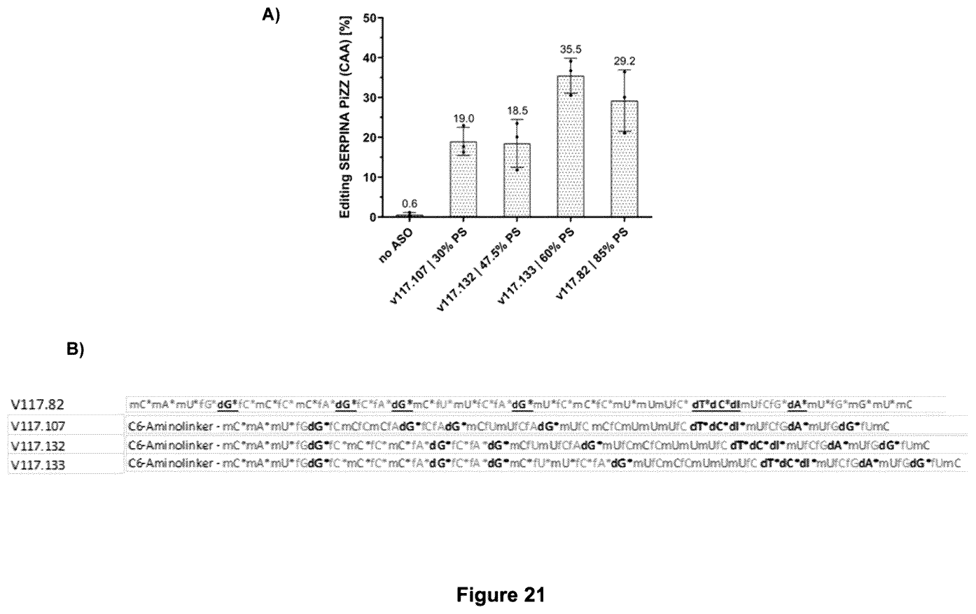

An ASO with no more than 3 continuous PS linkages (v117.107, 30% PS content) was tested and compared to an ASO with the same sequence and with either 10 (v117.132; 47.5% PS) or 16 (v117.133; 60% PS) continuous PS linkages. As shown in FIG. 21A, the 10 continuous PS linkages increased editing yield already but did not rescue the editing yield of the high-PS ASO v117.82. However, the 16 continuous PS linkages achieved editing levels similar to the editing levels of the PS-rich lead ASO (v117.82; with 25 continuous PS linkages, 85% PS). This highlights that the content of linkage modifications can be reduced (e.g., to at least 30%) for the short embodiments (≤50 nt), but that a continuous stretch of modified linkages (e.g., PS) is beneficial over a random spreading of modified linkages (e.g. PS) throughout the ASO. Furthermore, the data imply that PS content in these fully modified ASOs can be strongly reduced, which may be important when a reduction in toxicity is required.

Remarkably, many ASOs known from prior art (e.g., WO 2021/071858 and WO 2022/099159) contain a very high degree of (stereopure) phosphorothioate linkage modification to achieve editing yields in similar ranges. More importantly, their data is based on editing results in primary mouse hepatocytes, which generally provide high yields of editing and thus may give a limited picture.

Notably, there tends to be a certain threshold for linkage modifications. Hence, according to the invention, in one embodiment, linkage modification content (e.g., PS) is at least 30% to get the optimal editing yield. In one embodiment, the PS linkage modification content (e.g., PS) is at least 50%. In one embodiment, the PS content is at least 60%. Without being bound to any particular theory, short ASOs seem to favor more than 10 continuous linkage modifications (e.g., PS) over a dispersed pattern.

### Example 18. Improved Editing Efficacy and Potency of LNA-Modified SERPINA1 E342K Targeting ASOs

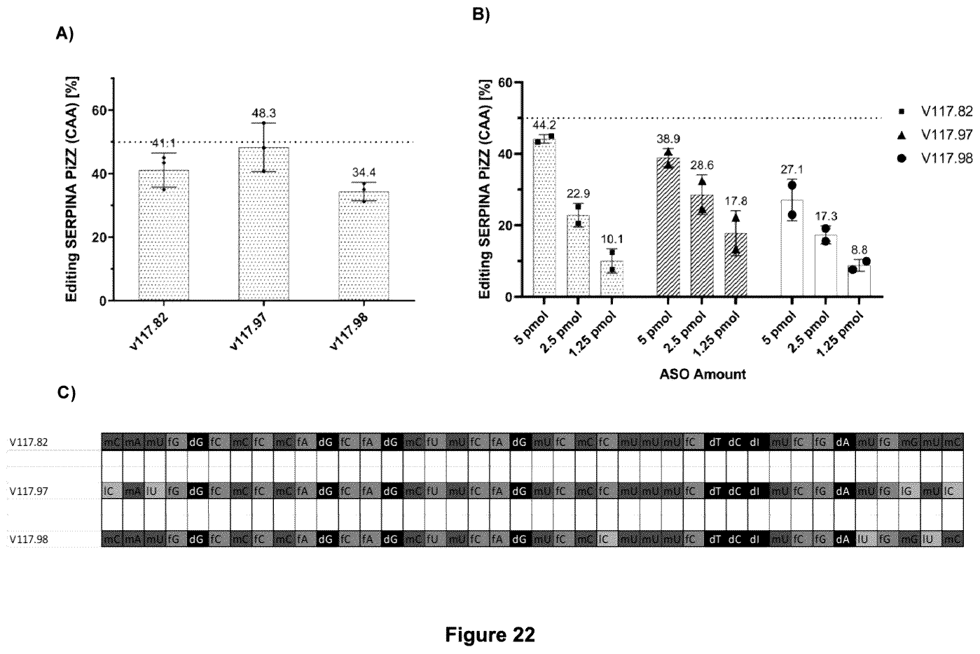

When shortening the ASOs, it was found that the 40 nt lead ASO can give a slightly reduced editing yield and potency. It is known from the art that LNAs provide for increased stability against enzymatic degradation and offer improved specificity and binding affinity in base-pairing. Hence, the 40 nt long ASO with 85% PS content (v117.82; “no LNA”) was modified by introducing LNA building blocks either at the 5′ and 3′ termini or within the oligonucleotide sequence. The different ASOs used in Example 17 are listed in Table 21, with the respective results shown in FIG. 22.

TABLE 21
SERPINA1 E342K targeting construct sequences and modifications used
in Example 18. IN = LNA, mN = 2′-O-methyl (2′-OMe), fN = 2′-fluoro (2′-F), N = 2′-OH
(ribose; RNA), dN = 2′-H (deoxyribose; DNA), * = phosphorothioate (PS) linkage.

| Construct | | Seq. | FIG. |
|---|---|---|---|
| Name | Construct Sequence (5′ to 3′ direction) | ID | No. |
| V117.82 | mC*mA*mU*fG*dG*fC*mC*fC*mC*fA*dG*fC*fA*dG*mC*fU*mU*fC*fA* | 63 | 22 |
| | dG*mU*fC*mC*fC*mU*mUmUfC* dT*dC*dI mU fC fG* dA* mU* | | |
| | fG*mG*mU*mC | | |
| V117.97 | IC*mA*IU*fG*dG*fC*mC*fC*mC*fA*dG*fC*fA*dG*mC*fU*mU*fC*fA*dG* | 134 | 22 |
| | mU*fC*mC*fC*mU*mUmUfC* dT*dC*dImUfCfG*dA*mU*fG*IG*mU*IC | | |
| V117.98 | mC*mA*mU*fG*dG*fC*mC*fC*mC*fA*dG*fC*fA*dG*mC*fU*mU*fC*fA* | 135 | 22 |
| | dG*mU*fC*mC*IC*mU*mUmUfC* | | |
| | dT*dC*dImUfCfG*dA*IU*fG*mG*IU*mC | | |
| V117.86 | mC*mA*mU*fG*dG* fC* mC* fC*mC* fA* dG* fC* fA* dG* mC* fU* mU* | 67 | 23 |
| | fC* fA*dG*mU*fC*mC*fC*mU*mUmUfC* dT*dC*dImUfCfG*dA*mU | | |
| V117.129 | C6-Aminolinker - | 136 | 23 |
| | IC*mA*IU*fG*dG*fC*mC*fC*mC*fA*dG*fC*fA*dG*mC*fU*mU*fC*fA*dG* | | |
| | mU*fC*mC*fC*mU*mUmUfC* dT*dC*dImUfCfG*dA*mU | | |
| V117.130 | C6-Aminolinker - | 137 | 23 |
| | IC*mA*IU*fG*IG*fC*IC*fC*mC*fA*dG*fC*fA*dG*mC*fU*mU*fC*fA*dG* | | |
| | mU*fC*mC*fC*mU*mUmUfC* dT*dC*dImUfCfG*dA*mU | | |
| V117.131 | C6-Aminolinker - | 138 | 23 |
| | IC*mA*IU*fG*IG*fC*IC*fC*IC*fA*IG*fC*fA*dG*mC*fU*mU*fC*fA*dG*mU* | | |
| | fC*mC*fC*mU*mUmUfC* dT*dC*dImUfCfG*dA*mU | | |

As shown in FIG. 22A, for construct v117.97, which contains a total of four terminal LNAs (two LNAs at each terminus separated by a 2′-OMe), LNA modification had little effect on the overall editing yield at high ASO dose compared to v117.82. However, the potency of v117.97 was slightly improved given clearly higher editing yields at 2.5 pmol and 1.25 pmol ASO dose (FIG. 22B). Placing the LNA modifications within the ASO closer to the CBT seemed to disturb the editing efficacy and potency of the ASO when compared to the ASO carrying LNA modifications at the 5′ and 3′ termini only (see, v117.97 vs v117.98).

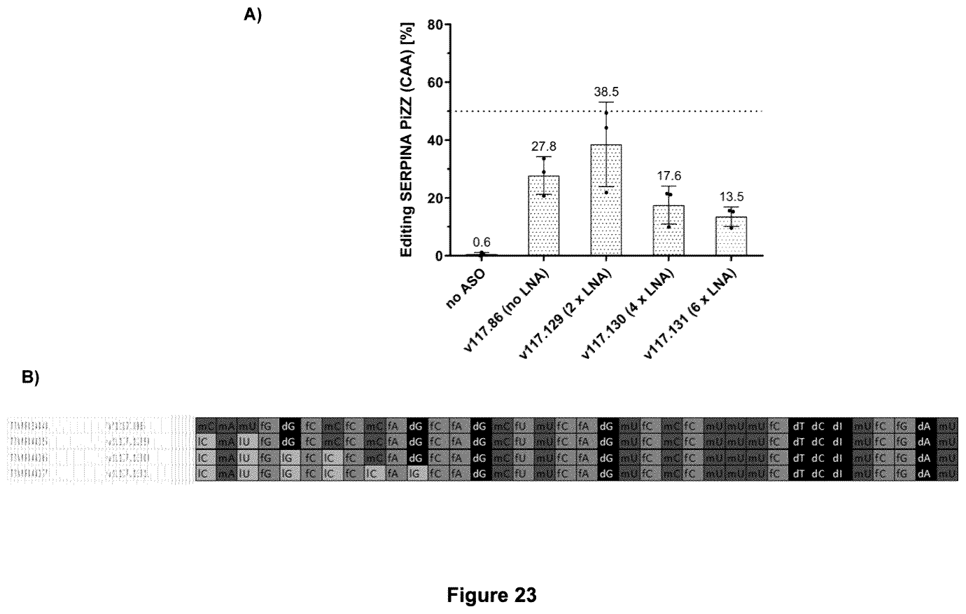

To determine the combined effect of terminal LNA modification(s) and ASO length on editing efficacy, LNA-modified versions (v117.129 to v117.131) of a shorter ASO (36 nt) with a PS content of 85% (v117.86; “no LNA”) were generated to comprise either 2, 4, or 6 LNA-modified nucleotides at their 5′ terminus as shown in Table 22 and depicted in FIG. 23B. The results are shown in FIG. 23A.

While two 5′ terminal LNAs were well accepted and improved overall editing of the ASO from 29% v117.86 to 49.4% v117.129, additional 5′ terminal LNAs interfered with editing yield (v117.130 and v117.131). Thus, without being bound by any theory, the inventors believe that short ASOs might benefit from 5′-terminal LNAs in small numbers.

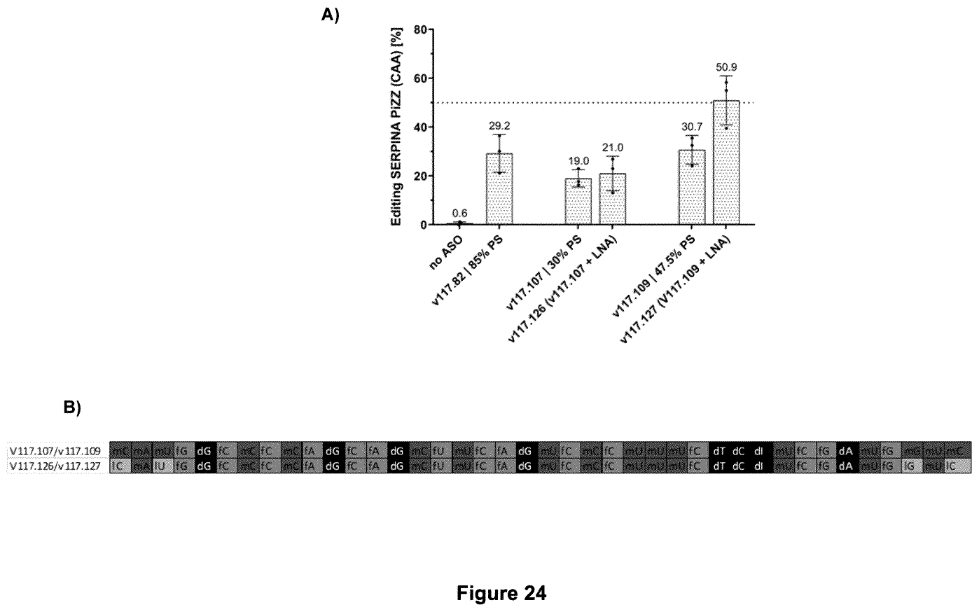

Lastly, the inventors assessed whether PS-reduced 40 nt ASOs with an overall PS content of about 30% (v117.107 and v117.126) and 48% (v117.109 and v117.127) do benefit from terminal LNAs. Different SERPINA1 E342K targeting constructs were generated to comprise a total of 4 LNAs (2 LNA modifications at each terminus interrupted by a 2′-OMe). The different constructs and their modifications are listed in Table 22 with the results being shown in FIG. 24A.

TABLE 22
SERPINA1 E342K targeting construct sequences and modifications used
in Example 18. IN = LNA, mN = 2′-O-methyl (2′-OMe), fN = 2′-fluoro (2′-F), N = 2′-OH
(ribose; RNA), dN = 2′-H (deoxyribose; DNA), * = phosphorothioate (PS) linkage.

| Construct | | Seq. | FIG. |
|---|---|---|---|
| Name | Construct Sequence (5′ to 3′ direction) | ID | No. |
| V117.82 | mC*mA*mU*fG*dG*fC*mC*fC*mC*fA*dG*fC*fA*dG*mC*fU*mU*fC*fA* | 63 | 24 |
| | dG*mU*fC*mC*fC*mU*mUmUfC* dT*dC*dImU fC fG* dA*mU* fG* | | |
| | mG* mU* mC | | |
| V117.107 | C6-Aminolinker - | 129 | 24 |
| | mC*mA*mU*fGdG*fCmCfCmCfAdG*fCfAdG*mCfUmUfCfAdG*mUfC | | |
| | mCfCmUmUmUfC dT*dC*dI*mUfCfGdA*mUfGdG*fUmC | | |
| V117.109 | C6-Aminolinker- | 131 | 24 |
| | mC*mA*mU*fG*dG*fCmCfCmCfA*dG*fCfA*dG*mCfUmUfCfA*dG* | | |
| | mU fC mC fC mU mU mU fC* dT*dC*dI*mU fC fG* | | |
| | dA*mUfG*dG*fUmC | | |
| V117.126 | C6-Aminolinker - | 139 | 24 |
| | IC*mA*IU*fGdG*fCmCfCmCfAdG*fCfAdG*mCfUmUfCfAdG*mUfCmCf | | |
| | CmUmUmUfC dT*dC*dI*mUfCfGdA*mUfGIG*fUIC | | |
| V117.127 | C6-Aminolinker - | 140 | 24 |
| | IC*mA*IU*fG*dG*fCmCfCmCfA*dG*fCfA*dG*mCfUmUfCfA*dG*mUfC | | |
| | mC fC mU mU mU fC* dT*dC*dI*mU fC fG*dA*mUfG*IG*fUIC | | |

Interestingly, it was found that the PS-reduced 40 nt short, SERPINA1 E342K targeting ASO benefited strongly from the 3′ and 5′ terminal LNAs (v117.127), achieving editing yields that were higher than those obtained with the 40 nt lead ASO with 85% PS, lacking terminal LNA modifications (v117.82). These data demonstrate that the overall editing efficacy and potency of the ASO can be improved through LNA modification, more specifically by placing a certain number of LNA (e.g., up to 6, more preferably less than 4) at the termini (e.g., preferably at the 5′ terminus) of the ASO (preferably in the short embodiment with a length of ≤50 nt). More importantly, the data show an improved performance of ASOs though LNA base modification(s) despite a reduced PS content. Without being bound by any particular theory, the inventors submit that LNAs might help to compensate for low PS content in short embodiments (≤50 nt, e.g., ≤40 nt).

### Example 19. Interruption of Continuous Blocks of 2′-Modifications in SERPINA1 E342K and STAT1 Targeting Constructs

ASOs carrying long stretches (or “continuous blocks”) of the same sugar modifications at the 2′ position (e.g., long blocks of 2′-O-methyl interrupted only by a CBT, or long blocks of 2′-F in combination with long blocks of 2′-O-methyl with interruption only at the CBT) are known in the art. Specifically, Monian et al. (2022) previously showed that chemically modified oligonucleotides (“AlMers”) with chimeric backbones containing continuous 2′-Fluoro- and/or 2′-OMe-modified backbones and a high degree of (stereopure) PS and PN linkages were able to mediate efficient in vitro target editing.

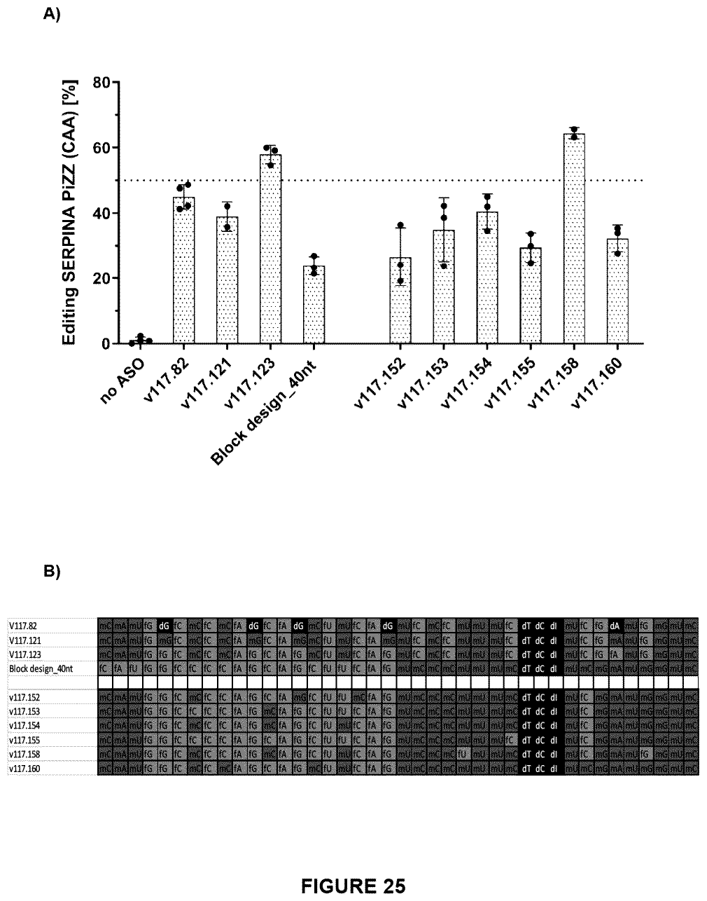

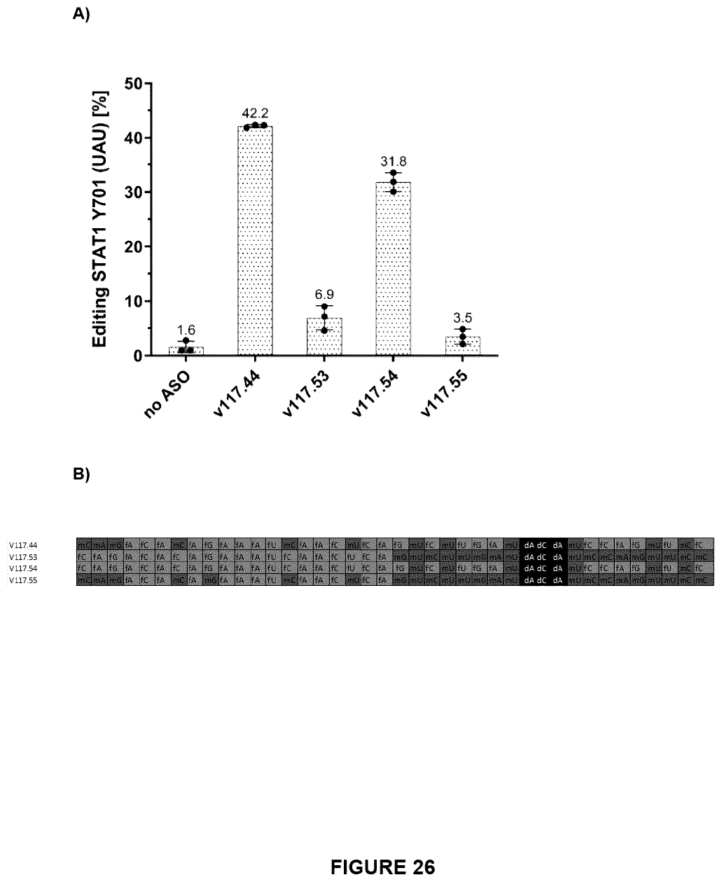

To determine the effect of disrupting such “continuous blocks”, e.g., continuous stretches of 2′-Fluoro- and/or 2′-OMe-backbone modifications, different SERPINA 1 E342K and STAT1 Y701 targeting constructs were generated and tested for their editing efficacy in vitro. The constructs used in Example 18 are listed in Tables 23 and 24 and the results are shown in FIGS. 25 and 26, respectively. “no ASO” served as negative control.

TABLE 23
SERPINA1 E342K targeting construct sequences and modifications used
in Example 19. mN = 2′-O-methyl (2′-OMe), fN = 2′-fluoro (2′-F), dN = 2′-H (deoxyribose;
DNA), * = phosphorothioate (PS) linkage.

| Construct | | Seq. | FIG. |
|---|---|---|---|
| Name | Construct Sequence (5′ to 3′ direction) | ID | No. |
| V117.82 | mC*mA*mU*fG*dG*fC*mC*fC*mC*fA*dG*fC*fA*dG*mC*fU*mU*fC*fA | 63 | 25 |
| | *dG*mU*fC*mC*fC*mU*mUmUfC* dT*dC*dImU fC fG* dA*mU* fG* | | |
| | mG* mU*mC | | |
| V117.121 | mC*mA*mU*fG*mG*fC*mC*fC*mC*fA*mG*fC*fA*mG*mC*fU*mU*fC* | 141 | 25 |
| | fA*mG*mU*fC*mC*fC*mU*mUmUfC* dT*dC*dImUfC fG* mA* mU* fG* | | |
| | mG*mU*mC | | |
| V117.123 | mC*mA*mU*fG*fG*fC*mC*fC*mC*fA*fG*fC*fA*fG*mC*fU*mU*fC*fA* | 142 | 25 |
| | fG*mU*fC*mC*fC*mU*mUmUfC* dT*dC*dImUfC fG* fA* mU* fG* | | |
| | mG*mU*mC | | |
| V117.152 | mC*mA*mU*fG*fG*fC*mC*fC*fC*fA*fG*fC*fA*mG*fC*fU*fU*mC*fA*fG | 143 | 25 |
| | *mU*mC*mC*mC*mU*mUmUmC* dT*dC*dI mUfCmG* mA* mU* | | |
| | mG* mG* mU* mC | | |
| V117.153 | mC*mA*mU*fG*fG*fC*fC*fC*fC*fA*fG*mC*fA*fG*fC*fU*fU*fC*fA*fG* | 144 | 25 |
| | mU*mC*mC*mC*mU*mUmUmC* dT*dC*dI mUfCmG* mA* mU* mG* | | |
| | mG* mU* mC | | |
| V117.154 | mC*mA*mU*fG*fG*fC*mC*fC*fC*fA*fG*mC*fA*fG*fC*fU*mU*fC*fA*fG | 145 | 25 |
| | *mU*mC*mC*mC*mU*mUmUmC* dT*dC*dI mUfC mG* mA* mU* | | |
| | mG* mG*mU*mC | | |
| V117.155 | mC*mA*mU*fG*fG*fC*fC*fC*fC*fA*fG*fC*fA*fG*fC*fU*fU*fC*fA*fG*mU | 146 | 25 |
| | *mC*mC*mC*mU*mUmUfC* dT*dC*dI mU fC mG* mA* mU*mG* | | |
| | mG* mU*mC | | |
| V117.158 | mC*mA*mU*fG*fG*fC*mC*fC*fC*fA*fG*mC*fA*fG*fC*fU*mU*fC*fA*fG | 147 | 25 |
| | *mU*mC*mC*mC*fU*mUmUmC* dT*dC*dI mU fC mG* mA* mU* fG* | | |
| | mG* mU* mC | | |
| V117.160 | mC*mA*mU*fG*fG*fC*mC*fC*mC*fA*fG*fC*fA*fG*mC*fU*mU*fC*fA* | 148 | 25 |
| | fG*mU*mC*mC*mC*mU*mUmUmC* dT*dC*dI mUmCmG* mA* mU* | | |
| | mG* mG* mU* mC | | |
| Block | fC*fA*fU*fG*fG*fC*fC*fC*fC*fA*fG*fC*fA*fG*fC*fU*fU*fC*fA*fG*mU* | 76 | 25 |
| design_40 nt | mC*mC*mC*mU*mUmUmC* dT*dC*dI mU mC mG* mA* mU* mG* | | |
| | mG* mU* mC | | |

As shown in FIG. 25, for SERPINA E342K targeting constructs, disruption of the continuous blocks of 2′-modifications (e.g., 2′-F and/or 2′-O-methyl) increased the overall editing efficacy of the ASOs. The data suggest a negative correlation between the block size of continuous 2′-modified blocks and the respective editing yields. As shown in FIG. 25A, a 40 nt control ASO (Block design_40 nt), containing 2′-F-blocks (e.g., 20 nt) and 2′-OMe blocks 5′ to the CBT (e.g., 8 nt) and 3′ to the CBT (e.g., 9 nt) showed a low editing efficacy of around only 20%. Compared to that, the highest editing yields were obtained for v117.123 and v117.158 (achieving around 60% editing yields), both of which contain interruptions of the 2′-F blocks by single 2′-O-methyl modifications (e.g., largest 2′-F block size 5 nt) and interruptions of the two 2′-O-methyl blocks by single 2′-F modifications or small blocks (e.g., 3 nt) of 2′-F modification (e.g., largest 2′-O-methyl block size 4 nt). This suggests that interrupted blocks are very important for optimal editing efficiency.

The data further show that DNA nucleosides can be used to disrupt either 2′-F blocks or 2′-O-methyl blocks (v117.82). While, in this case, the DNA might not have been ideally placed, resulting in some loss of editing efficiency, it still performed better than the Block design_40 nt control. Furthermore, the data show that relatively large 2′-F blocks and 2′-O-methyl blocks can be accepted at the 5′-half of the ASO (v117.155). However, the editing yields were clearly lower than for those constructs with smaller 2′-F and/or 2′-O-methyl block sizes (e.g., v117.121, v117.123 and, v117.158 versus v117.153, v117.154,) v117.158). The low performance (e.g., editing yields around 25-40%) of constructs v117.152, v117.153 and v117.154 when compared to the two best embodiments in this data set (e.g., v117.158, and v117.123, with editing yields of around 60%) demonstrate the importance of combining block disruption in the large 2′-F block with disruption in the two large 2′-OMe blocks surrounding the CBT. Also, these embodiments show that high amounts of 2′-OMe (55% in v117.121) can be well tolerated and even improve editing yields when replacing DNA nucleosides (approx. 60% editing yields for v117.121 versus 40% editing yields for v117.82).

Analogously, STAT1 Y701 targeting ASOs, carrying disruptions of continuous block modifications, were generated and tested for their RNA editing efficacy. The STAT1 Y701 targeting constructs are listed in Table 24 and the results are shown in FIG. 26. “no ASO” served as negative control.

TABLE 24
STAT1 Y701 targeting construct sequences and modifications used in
Example 19. mN = 2′-O-methyl (2′-OMe), fN = 2′-fluoro (2′-F), dN = 2′-H (deoxyribose;
DNA), * = phosphorothioate (PS) linkage.

| Construct | | Seq. | FIG. |
|---|---|---|---|
| Name | Construct Sequence (5′ to 3′ direction) | ID | No. |
| V117.44 | mC*mA*mG*fA*fC*fA*mC*fA*fG*A*fA*fA*fU*mC*fA*fA*fC*mU*fC*fA*fG | 84 | 26 |
| | *mU*fC*mU*fU*fGfAmU* dA*dC*dA mUfC fC*fA*fG*mU*fU*mC*fC | | |
| V117.53 | fC*fA*fG*fA*fC*fA*fC*fA*fG*fA*fA*fA*fU*fC*fA*fA*fC*fU*fC*fA*mG*mU* | 149 | 26 |
| | mC*mU*mU*mGmAmU* dA*dC*dA mUmC mC* mA* mG* mU* mU* | | |
| | mC* mC | | |
| V117.54 | fC*fA*fG*fA *fC*A*fC*fA*fG*fA*fA*fA*fU*fC*fA*fA*fC*fU*fC*fA* fG* mU* | 150 | 26 |
| | fC* mU*fU*fG fA mU* dA*dC*dA mUfC fC*fA*fG*mU*fU*mC*fC | | |
| V117.55 | mC*mA*mG*A*fC*fA*mC*fA*mG*A*fA*fA*fU*mC*fA*fA*fC*mU*fC*fA* | 151 | 26 |
| | mG*mU*mC*mU*mU*mGmAmU* dA*dC*dA mUmC mC* mA* mG* | | |
| | mU* mU* mC*mC | | |

STAT1 Y701 targeting ASOs were based on the short embodiment (≤50 nt, e.g., 40 nt), wherein the ASOs comprise less than or equal to three (≤3 nt) consecutive 2′-OMe modifications and less than or equal to six (≤6 nt) consecutive 2′-F-modifications (v117.44). This embodiment gave the best editing result in the dataset (42%). This was compared to a control ASO with a large 5′ terminal 2′-F block (20 nt) and two large 2′-O-methyl blocks 3′ (8 nt) and 5′ (9 nt) to the CBT (see, v117.53). This version performed very badly achieving a low editing yield of only 7%, close to the Sanger sequencing detection limit. With a domain swap experiment we the inventors showed for this target sequence that block interruptions in the 2′-O-methyl blocks were particularly important. This is demonstrated particularly for v117.54, which still contains the large 2′-F block, and still performed well compared to v117.55, which contains disrupted block disruptions at both the 2′-F blocks with and the two large, continuous 2′-O-methyl blocks.

This demonstrates that large 2′-F blocks are likely better accepted than large 2′-O-methyl blocks. However, the best performing embodiment is characterized by a maximum continuous block size of 6 nt (v117.44).

### Example 20. 2′-FANA Modification within the CBT of CRB1 C948Y Targeting Constructs

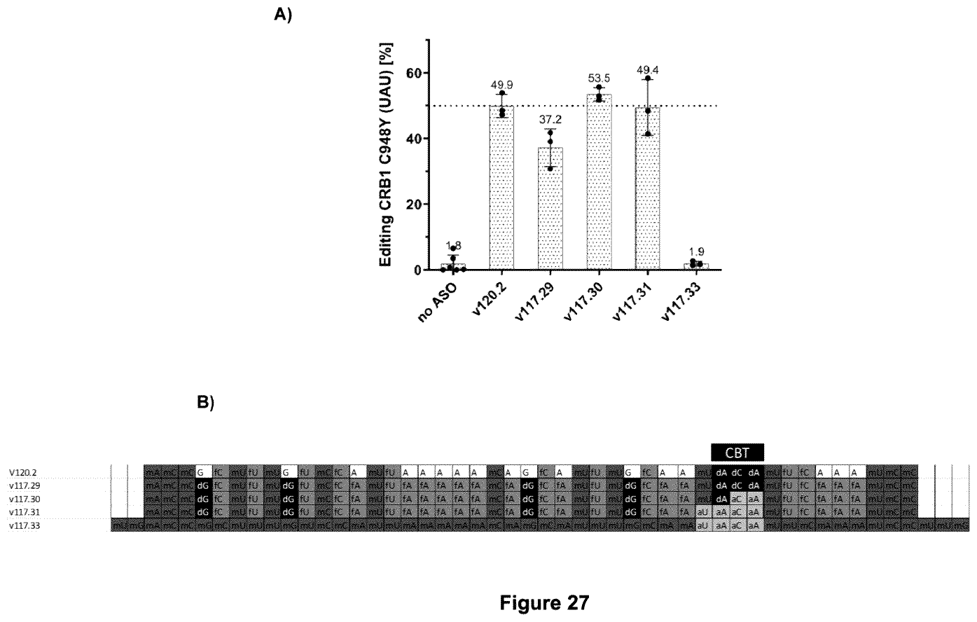

It has previously been shown that 2′-FANA modification within the CBT (WO 2021/243023), and at specifically at the N₊₁ position, has a positive effect on ASO-mediated on-target editing (Brinkman et al., 2022). To determine the effect of 2′-FANA modification within and 5′ to the CBT, 2′-FANA modifications were introduced in two different patterns at the N₋₂, N₋₁, No, and/or N₊₁ positions of CRB1 C948Y targeting constructs. The various constructs and their modifications used in Example 19 are listed in Table 25. The corresponding results are shown in FIG. 27. “no ASO” was used as negative control.

TABLE 25
CRB1 C948Y targeting construct sequences and modifications used in
Example 20. mN = 2′-O-methyl (2′-OMe), fN = 2′-fluoro (2′-F), dN = 2′-H (deoxyribose;
DNA), * = phosphorothioate (PS) linkage; aN = 2′-FANA.

| Construct | | Seq. | FIG. |
|---|---|---|---|
| Name | Construct Sequence (5′ to 3′ direction) | ID | No. |
| V120.2 | mA*mC*mC*G*fC*mU*fU*mU*G*fU*mC*fC*A*mU*fU*A*A*A*A*A*mC*A | 92 | 27 |
| | *G*fC*A*mU*fU*mU*G*fC* AAmU* dA*dC*dA | | |
| | mUfUfC*A*A*A*mU*mC*mC | | |
| V117.29 | mA*mC*mC*dG*fC*mU*fU*mU*dG*fU*mC*fC*fA*mU*fU*fA*fA*fA*fA*fA | 96 | 27 |
| | *mC*fA*dG*fC*fA*mU*fU*mU*dG*fC* fAfAmU* dA*dC*dA mUfUfC*fA* | | |
| | fA* fA* mU* mC* mC | | |
| V117.30 | mA*mC*mC*dG*fC*mU*fU*mU*dG*fU*mC*fC*fA*mU*fU*fA*fA*fA*fA*fA | 152 | 27 |
| | *mC*fA*dG*fC*fA*mU*fU*mU*dG*fC* fAfAmU* dA*aC*aA mUfUfC | | |
| | *fA*fA*A*mU*mC*mC | | |
| V117.31 | mA*mC*mC*dG*fC*mU*fU*mU*dG*fU*mC*fC*fA*mU*fU*fA*fA*fA*fA*fA | 153 | 27 |
| | *mC*fA*dG*fC*fA*mU*fU*mU*dG*fC* fAfAaU* aA*aC*aA mUfUfC *fA* | | |
| | fA*fA*mU*mC*mC | | |
| V117.33 | mU*mG*mA*mC*mCmGmCmUmUmUmGmUmCmCmAmUmUmAmA | 154 | 27 |
| | mAmAmAmCmAmGmCmAmUmUmUmGmC mAmAaU aAaCaA | | |
| | mUmUmC | | |

As shown in FIG. 27A, one or more 2′-FANA-modifications within and/or 5′ to the CBT together with a combination of 2′-F and/or 2′-OMe-modifications outside the CBT significantly increased the overall editing efficacy when compared to control, highlighting that FANA is more than a mere alternative to DNA, 2′-F and 2′-O-methyl inside the CBT in this embodiment.

The sole introduction of 2′-F, 2′-OMe-, and 2′-H modifications led to a slight decrease in editing efficacy relative to the control containing 17 natural RNA nucleosides (see, v117.29 vs v120.2). However, introducing 2′-FANA-modifications at the No and N₊₁ positions of the fully 2′-modified embodiment (v117.30; two×2′-FANA at the CBT) resulted in an increase in editing efficacy back to levels of the RNA-rich control ASO v120.2, thereby clearly outcompeting the fully 2′modified embodiment lacking FANA (v117.29; no 2′-FANA). Addition of additional 2′-FANA modifications to the N_{-Z} and N₋₁ positions was well accepted but did not further enhance the editing efficacy of the ASO (v117.31) when compared to v117.30 (two 2′-FANA at the CBT). Interestingly, a control ASO (v117.33), which comprises an additional two nucleosides at the 5′ terminus and an additional 3 nucleotides at the 3′ terminus relative to v117.30, showed a drastic decrease in editing efficacy below the Sanger sequencing detection limit (below 5%). This control construct was based on an embodiment previously disclosed in the prior art. That is, the 2′-sugar and linkage modification pattern is identical to the sequence KB-018-698 listed in patent WO 2021/243023. In the present case, the nucleobase sequence was changed to match the CRB1 C948Y site by copying the modification framework and sequence symmetry of KB-018-698 and transferring it onto another target transcript/site. As expected, the uniform modification with 2′-O-methyl outside the CBT, which is a hallmark of v117.33, strongly interferes with the RNA editing efficiency efficacy and cannot be rescued by the presence of FANA in the CBT or by the extended length of the ASO (which is 50 nt), highlighting again the importance of combining optimal linkage and 2′ modifications patterns (2′-O-methyl, 2′-F and/or DNA), right positioning of the ASO (asymmetric for short embodiments ≤50 nt) and limited block size (e.g., ≤6 nt) of uniform 2′-modification (e.g., 2′-O-methyl, but also 2′-F and DNA).

These data suggest that a combination of 2′-FANA-modifications at and 5′ to the CBT in combination with 2′-F- and 2′-OMe-modifications can be used to stabilize the ASO and to maintain or increase their editing efficacy. Generally, ASOs comprising a combination of 2′-F- and 2′-OMe-modifications and DNA were able to tolerate a total of at least four 2′-FANA modifications.

In summary, the inventors have shown that a balanced mixture of modifications at the 2′ position of the sugar moiety of the oligonucleotide (e.g., at least 10% (preferably 20-70%) 2′-F-, at least 10% (preferably 20-60%) 2′-OMe-, sometimes 2′-H (not more than 50% in long, e.g., ≥40 ntASOs, and not more than 6 DNA in short, e.g., ≤50 ntASOs), 2′-OH, etc.) enables stabilization in a lysosomal surrounding, thus protecting them against nuclease digestion, e.g., during uptake, while preserving high levels of editing efficiency efficacy. Specifically, the inventors have successfully shown that mixtures of such modifications together with specifically placed internucleoside linkage modifications (e.g., linkage modification at linkage d and e, but no PS modification at linkages h and i) provide a way of generating effective and stable ASOs. Particularly, the inventors have shown that depending on the length of the ASO, positioning of these specific 2′-modifications at particular sites within the ASO can have a significant impact on the overall editing efficacy of the ASO. Moreover, the inventors have identified an extended hotspot region (5′-CBT-mN-fN-3′). In cases where the positioning of the 2′-modification(s) is less important, the combination of chemical modifications is crucial. Furthermore, while the inventors have shown that certain levels of 2′-F and 2′-OMe modifications should be included (“extended hotspot”), continuous blocks of uniform sugar modifications should clearly be avoided (e.g., ≤6 nt for 2′-F, 2′-O-methyl and/or DNA, with 2′-F being best accepted in larger uniform blocks). Moreover, it has been shown that placement of these modifications is somewhat position-independent and that the specific modifications and/or modification patterns can be transferred, to some extent, in a position-specific manner to different ASOs of distinct target specificities. The inventors have shown that at least for embodiments of this invention, FANA modifications within the CBT are well accepted at specific positions and can improve editing yields over CBTs built from DNA only. Furthermore, the inventors have successfully shown that PS content can be strongly reduced (down to 15%), in particular for long embodiments (≥40 nt); and that short embodiments (≤50 nt) benefit from longer continuous stretches of (e.g., one stretch of 10) linkage modifications (e.g., PS) and/or a combination with (terminal) LNA modifications, or a generally slightly higher degree of linkage modification (e.g., at least 30%, e.g., PS).

Those having ordinary skill in the art will appreciate that the disclosure can be modified in ways not specifically described herein.

1. A chemically modified oligonucleotide comprising a sequence with a length of 23 to 80 nucleotides, capable of binding to a target sequence in a target RNA, comprising a central base triplet (CBT) of 3 nucleotides (5′-N₋₁^{e}N₀^{f}N₊₁^{g}-3′) with the central nucleotide (N₀) directly opposite to the target adenosine in the target RNA, wherein the core oligonucleotide comprises the following sequence:
5′ - N₋₅ ª N₋₄ ^{b} N₋₃ ^{c} N₋₂ ^{d} N₋₁ ^{e} N₀ ^{f} N₊₁ ^{g}
N₊₂ ^{h} N₊₃ ⁱ N₊₄ ^{j} -3′;
and
wherein:
(a) at least two of the three nucleotides of the CBT are chemically modified at the 2′ position of the sugar moiety, are deoxyribonucleosides, or a combination thereof and wherein d and e are internucleoside linkage modifications;
(b) the N₊₂ nucleotide carries a 2′-O-alkyl-modification; and wherein the N₊₃ nucleotide carries a 2′-fluoro (2′-F)-modification;
(c) at least 10% of nucleotides are 2′-F-modified and at least 10% of nucleotides are 2′-O-alkyl-modified, wherein no more than 6 consecutive nucleotides have the same 2′-modification;
(d) the internucleoside linkage modification content is at least 15%; and
(e) linkages h and i are not phosphorothioate (PS) linkages.

2. The chemically modified oligonucleotide according to claim 1, wherein 20-100% of nucleotides are deoxyribonucleosides or 2′-modified, preferably wherein 50-100% of nucleotides are 2′-modified nucleotides.

3. The chemically modified oligonucleotide according to claim 1, wherein 20-70% of nucleotides are 2′-F-modified, preferably wherein 35-65% of nucleotides are 2′-F-modified; and/or
wherein 20-60% of nucleotides are 2′-O-methyl (2′-OMe)-modified, preferably wherein 25-55% of nucleotides are 2′-OMe-modified.

4. The chemically modified oligonucleotide according to any claim 1, wherein
(i) no more than 95%, 90%, 85%, 80%, 70%, 60%, 50%, 40%, 30% or 20% of the linkages outside the CBT are internucleoside linkage modifications; or
(ii) 15-90% of the linkages are internucleoside linkage modifications, preferably wherein 40-80%, most preferably 45-60%, of the linkages are internucleoside linkage modifications.

5. (canceled)

6. The chemically modified oligonucleotide according to claim 1, wherein the oligonucleotide has a length of:
(i) 23-50, 28-70, 28-60, 28-55, or 28-45 nucleotides;
(ii) 59 nucleotides; or
(iii) no more than 45 nucleotides.

7. The chemically modified oligonucleotide according to claim 1, wherein the oligonucleotide has a length of 45 or less nucleotides and wherein outside of the CBT no more than 4 nucleotides are deoxyribonucleotides.

8. A chemically modified oligonucleotide comprising a sequence with a length of 23 to 50 nucleotides, capable of binding to a target sequence in a target RNA, comprising a central base triplet (CBT) of 3 nucleotides (5′-N₋₁^{e}N₀^{f}N₊₁^{g}-3′) with the central nucleotide (N₀) directly opposite to the target adenosine in the target RNA, wherein the core oligonucleotide comprises the following sequence:
5′ - N₋₅ ª N₋₄ ^{b} N₋₃ ^{c} N₋₂ ^{d} N₋₁ ^{e} N₀ ^{f} N₊₁ ^{g}
N₊₂ ^{h} N₊₃ ⁱ N₊₄ ^{j} -3′;
and
wherein:
(a) at least two of the three nucleotides of the CBT are chemically modified at the 2′ position of the sugar moiety, are deoxyribonucleosides, or a combination thereof and wherein d and e are internucleoside linkage modifications;
(b) the N₊₂ nucleotide carries a 2′-O-alkyl-modification; and wherein the N₊₃ nucleotide carries a 2′-fluoro (2′-F)-modification;
(c) at least 10% of nucleotides are 2′-F-modified and at least 10% of nucleotides are 2′-O-alkyl-modified, wherein no more than 6 consecutive nucleotides have the same 2′-modification;
(d) the regions 3′ and 5′ to the CBT do not contain more than a total of 6 deoxyribonucleosides; and
(e) the internucleoside linkage modification content is at least 30%.

9. The chemically modified oligonucleotide according to claim 1, wherein outside of the CBT,
(i) the oligonucleotide does not contain any deoxyribonucleosides, or
(ii) no more than 1, 2, 3, or 4 nucleotides are deoxyribonucleotides.

10. The chemically modified oligonucleotide according to claim 8, wherein the internucleoside linkage modification content is between 30-90%.

11. A chemically modified oligonucleotide comprising a sequence with a length of 40 to 80 nucleotides, capable of binding to a target sequence in a target RNA, comprising a central base triplet (CBT) of 3 nucleotides (5′-N₋₁^{e}N₀^{f}N₊₁^{g}-3′) with the central nucleotide (N₀) directly opposite to the target adenosine in the target RNA, wherein the core oligonucleotide comprises the following sequence:
5′ - N₋₅ ª N₋₄ ^{b} N₋₃ ^{c} N₋₂ ^{d} N₋₁ ^{e} N₀ ^{f} N₊₁ ^{g}
N₊₂ ^{h} N₊₃ ⁱ N₊₄ ^{j} -3′;
and
wherein:
(a) at least two of the three nucleotides of the CBT are chemically modified at the 2′ position of the sugar moiety, are deoxyribonucleosides, or a combination thereof and wherein d and e are internucleoside linkage modifications;
(b) the N₊₂ nucleotide carries a 2′-O-alkyl-modification; and wherein the N₊₃ nucleotide carries a 2′-fluoro (2′-F)-modification;
(c) at least 10% of nucleotides are 2′-F-modified and at least 10% of nucleotides are 2′-O-alkyl-modified, wherein no more than 6 consecutive nucleotides have the same 2′-modification;
(d) the regions 3′ and 5′ to the CBT have a total deoxyribonucleoside content of 5-50%.

12. The chemically modified oligonucleotide of claim 11, wherein the deoxyribonucleoside content outside the CBT is 10-40%, more preferably 11-30%, and even more preferably 13-25%.

13. The chemically modified oligonucleotide according to claim 1, comprising at least one internucleoside linkage modification selected from the group consisting of phosphorothioate (PS), 3′-methylenephosphonate, 5′-methylenephosphonate, 3′-phosphoroamidate, 2′-5′phosphodiester, and phosphoryl guanidine (PN).

14. The chemically modified oligonucleotide according to claim 13, wherein the at least one internucleoside linkage modification is PS.

15. The chemically modified oligonucleotide according to claim 8 linkages h and i are not phosphorothioate (PS) linkages.

16. The chemically modified oligonucleotide according to claim 1, wherein linkages h and i are phosphate (PO) linkages.

17. The chemically modified oligonucleotide according to claim 1, wherein
(i) no more than 4, 5, or 6 consecutive nucleotides are 2′-F-modified; and/or
(ii) no more than 4, 5, or 6 consecutive nucleotides are 2′-O-alkyl-modified.

18. The chemically modified oligonucleotide according to claim 1, wherein less than 6, 5, 4, or 3 consecutive nucleotides have the same 2′-modification.

19. The chemically modified oligonucleotide according to claim 1, wherein the chemically modified oligonucleotide comprises one or more stereorandom internucleoside linkage modifications.

20. The chemically modified oligonucleotide according to claim 1, wherein the chemically modified oligonucleotide comprises no more than 10 stereopure internucleoside linkages.

21. The chemically modified oligonucleotide according to claim 20, wherein the stereopure linkages are PS linkages and/or PN linkages.

22. The chemically modified oligonucleotide according to claim 1, wherein the chemically modified oligonucleotide comprises no stereopure PS linkages and/or no stereopure PN linkages.

23. (canceled)

24. The chemically modified oligonucleotide according to claim 1, wherein the oligonucleotide comprises:
(i) 2′-O-(2-Methoxyethyl)-oligoribonucleotide (2′-MOE) terminal blocks at the 3′ and 5′ termini, wherein at each terminus there are no more than 4 nucleotides with 2′-MOE, preferably no more than 3 nucleotides with 2′-MOE; and/or
(ii) terminal locked nucleic acids (LNAs), wherein the oligonucleotide comprises 2 to 6 LNAs at each terminus or the 5′ terminus; preferably wherein the oligonucleotide comprises 2 LNAs at each terminus or the 5′ terminus.

25. The chemically modified oligonucleotide according to claim 1, wherein linkage g is not a PS linkage, preferably wherein linkage g is a phosphate (PO) linkage.

26. The chemically modified oligonucleotide according to claim 1, wherein d and e are PS linkage modifications, optionally wherein f is an internucleoside linkage modification.

27. The chemically modified oligonucleotide according to claim 1, wherein the modification at the 2′-position of subsection (a) is a
(i) 2′-O-alkyl-modification,
(ii) 2′-F-modification, or
(iii) 2′-fluoroarabinoside (FANA)-modification.

28. The chemically modified oligonucleotide according to claim 1, wherein the 2′-O-alkyl-modification is a 2′-OMe-modification.

29. The chemically modified oligonucleotide according to claim 1, wherein each of the three nucleosides of the CBT is either singularly or a combination of:
(i) a deoxyribonucleotide; and/or
(ii) 2′-FANA-modification; and/or
(iii) 2′-O-methyl-modification; and/or
(iv) 2′-F-modification.

30. The chemically modified oligonucleotide according to claim 1, wherein
(i) N₋₁ is 2′-F, 2′-FANA, DNA, or 2′-O-methyl; and/or
(ii) N₀ is 2′-FANA or DNA or deoxycytidine or FANA-cytidine; and/or
(iii) N₊₁ is 2′-FANA, DNA, or 2′-O-methyl.

31. (canceled)

32. The chemically modified oligonucleotide according to claim 1, wherein positions −5, −4, and −3 are 2′-O-alkyl-modified; and/or wherein position −2 is 2′-F-modified.

33. The chemically modified oligonucleotide according to claim 1, wherein there are:
(i) at least 4 nucleotides 3′ of the CBT; and/or
(ii) at least 16 nucleotides 5′ of the CBT.

34. The chemically modified oligonucleotide according to claim 1, comprising
(i) at least 10 continuous internucleoside linkage modifications; and/or
(ii) 3 consecutive internucleoside linkage modifications at each terminus.

35. The chemically modified oligonucleotide according to claim 1, wherein the chemically modified oligonucleotide does not comprise a loop-hairpin structured ADAR recruitment motif.

36. The chemically modified oligonucleotide according to claim 1, wherein the chemically modified oligonucleotide comprises one or more mismatches, wobble base, and/or bulges with respect to its target, and/or a mismatch at N₀.

37. A pharmaceutical composition comprising the oligonucleotide of according to claim 1.

38. A method of treating or preventing a genetic disorder, condition, or disease in a subject, the method comprising administering to the subject in need thereof an effective amount of the chemically modified oligonucleotide of claim 1.

39. The method of claim 38, wherein the genetic disorder, condition or disease is selected from the group consisting of: Retinitis pigmentosa (RP), Stargardt macular degeneration, age-related macular degeneration (AMD), Cystic fibrosis (CF), Hurler Syndrome, alpha-1-antitrypsin (A1AT) deficiency, Parkinson's disease, Alzheimer's disease, albinism, Amyotrophic lateral sclerosis, Asthma, β-thalassemia, Cadasil syndrome, Charcot-Marie Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis (LCA), Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders.

40.-41. (canceled)

42. An in vitro method for editing at least one target adenosine in a target nucleic acid, wherein the method comprises contacting the target nucleic acid with the oligonucleotide of claim 1.

43.-44. (canceled)
